(19) United States
(12) Patent Application Publication
(10) Pub. No.: US 2013/0226268 A1
(43) Pub. Date: Aug. 29, 2013

(54) NANOCRYSTALS IN DEVICES

(71) Applicant: Junyou Pan, Frankfurt am Main (DE)

(72) Inventor: Junyou Pan, Frankfurt am Main (DE)

(73) Assignee: Merck Patent GmbH, Darmstadt (DE)

(21) Appl. No.: 13/810,486

(22) PCT Filed: Jun. 24, 2011
(86) PCT No.: PCT/EP2011/003133

§ 371 Date: May 20, 2013

(30) Foreign Application Priority Data
Jul. 26, 2010 (EP) 10007742.9

(51) Int. Cl. A61N 5/06; A23L 3/005; A23L 3/26; A61L 2/08

(52) U.S. Cl. CPC A61N 5/0616; A61L 2/08; A23L 3/005; A23L 3/26
USPC 607/88; 252/519.2; 422/291; 422/22; 426/237

(57) ABSTRACT

The present invention relates inter alia to devices comprising quantum dots, ionic species, and further organic functional materials, their preparation and use.

The present invention relates inter alia to devices comprising at least one quantum dot and at least one small molecule organic functional material. The present invention also relates to the use of the device in, e.g., therapeutic and/or cosmetic, information display and general lighting applications.

Phototherapy (also called light therapy) can be employed in a wide range of therapeutic diseases and/or cosmetic (also called aesthetic) conditions. Phototherapy by either employing LED or laser as light source has already been used to treat wounds, injuries, neck pain, osteoarthritis, the side effects of chemotherapy and radiotherapy, for instance.

Often the borders between therapeutic and cosmetic applications are vague and depend on individual circumstances and the assessment a physician. Often therapeutic conditions are associated with cosmetic consideration and vice versa. The treatment or prophylaxis of acne, for example, may have both therapeutic and cosmetic components, depending on the degree of the condition. The same accounts for psoriasis, atopic dermatitis and other diseases and/or conditions. Many diseases and conditions are associated with apparent implications which are often represented by a change in the visibility of a subject's skin, for instance. These cosmetic or aesthetic changes can often lead to psychological modifications resulting, at least in part, in serious diseases.

Some conditions or diseases may have an emphasis on cosmetic components, even if therapeutic elements may also play a role. Some of these are selected from anti-ageing, anti-wrinkle, the prevention and/or therapy of acne and vitiligo.

Many diagnostic tools or devices also often require light sources, e.g., in order to determine blood characteristics such as bilirubin, oxygen, or CO. In both cosmetics and medicine the skin is the main target to be irradiated, but other targets of the human or animal body can also be accessed by phototherapy. These targets include, but are not limited to, the eye, wounds, nails, and internal parts of the body. Light can also be used in order to facilitate or support disinfection of wounds, beverages, nutrition, for example.

One effect of phototherapy is the stimulation of metabolism in mitochondria. Certain wavelengths of light stimulate cytochrome c oxidase, an enzyme which is responsible for the production of the essential cellular energy in the form of adenosine triphosphate (ATP). ATP is required for cellular energy transfer in order to drive thermodynamically unfavoured biochemical reactions and as cellular energy storage. ATP can also act as signal molecule in order to modulate other biochemical molecules (e.g. reactive oxygen species and nitric oxide) that lead to ageing and cell death (oxidative stress). After phototherapy, the cells show an increased metabolism, they communicate better and they survive stressful conditions in a better way.

Such principle can be applied for medicinal therapeutic and cosmetic applications, such as wound healing, connective tissue repair, tissue repair, prevention of tissue death, relief of inflammation, pain, acute injuries, chronic diseases, metabolic disorders, neurogenic pain and seasonal effect disorders.

Another area of the application of light is the treatment of various cancers. In cancer therapy photodynamic therapy (PDT) plays an important role. In PDT light may be used in conjunction with a pharmaceutical. These therapies can be used to treat a variety of skin and internal diseases. In PDT, a light-sensitive therapeutic agent known as a photopharmaceutical is supplied externally or internally to an area of the body which is to be treated. That area is then exposed to light of a suitable frequency and intensity to activate the photopharmaceutical. A variety of photopharma-ceutical agents are currently available. For example there are topical agents such as 5-aminolevulinic acid hydrochloride (Crawford Pharma-ceuticals), methylaminolevulinic acid (Metfix®, Photocure). There are also injectable drugs used primarily for internal malignancies, including Photofin® (from Axcan) and Foscan® (from Biolitech Ltd). Often, the drug is applied in a non-active form that is metabolised to a light-sensitive photopharmaceutical.

In photodynamic therapy, the primary technique for supplying light to the photopharmaceutical is to project light of a suitable wavelength from standalone light sources such as lasers or filtered arc lamps. These sources are cumbersome and expensive, and are therefore only suitable for use in hospitals. This leads to inconvenience for the patient, and high cost for the treatment. High light irradiances are needed in order to treat an acceptable number of patients per day (for the treatment to be cost effective) and to avoid unduly inconveniencing the patient.

To date, phototherapy and PDT is dominated by the application of large light sources being uncomfortable for patients leading to low compliance. Many of the devices which are currently in use are only applicable stationary and require the control of medical professionals, e.g. in hospital or in doctor's surgery. Furthermore, many of the light sources currently used irradiate large areas of the subject to be treated, even if only a fraction of it should have been irradiated which may lead to unwanted side effects.

WO 98/46130 and U.S. Pat. No. 6,096,066 disclose arrays of LEDs for use in photodynamic therapy. The small LED sources taught therein result in uneven light incident on the patient. Fabrication of arrays is complicated because of the large number of connections required. The devices shown therein are designed for hospital treatment.

GB 2360461 discloses a flexible garment which uses a conventional photodynamic therapy light source to produce light which is then transmitted through optical fibres. As such light sources are heavy, the device is not ambulatory and is limited to hospital use.

U.S. Pat. No. 5,698,866 discloses a light source using over-driven inorganic LEDs. The resulting light output is not even. A heat-sinking mechanism is required, and the device is suitable only for hospital treatment.

WO 93/21842 discloses light sources using inorganic LEDs. Although transportable, the device is not suitable for ambulatory use by a patient at home and clinical treatment is envisaged.

A further problem with existing approaches is that it can be difficult to achieve uniform illumination with such sources, especially on curved body parts.

An essential prerequisite for the application of light in the fields mentioned above is the device. The commercial available systems nowadays are mostly based on lasers. However, theses systems are hospital based, i.e. stationary devices. In order to reduce costs and to increase convenience as well as compliance a portable home-use technology is required. In fact, some research has been devoted in this direction.

Rochester et al. disclosed in GB 24082092 a flexible medical light source comprising flexible light emitting diodes form on flexible substrate and resulting diagnostic devices directed to monitoring blood characteristics (e.g. levels of CO, oxygen, or bilirubin) and photo-therapeutic devices for treatment of ailments.

Vogle Klaus and Kallert Heiko disclosed in EP 018180773 a device for the treatment of skin. The device comprises an potentially flexible organic light emitting diode (OLED) as light source. The device can be integrated in clothes or plaster.

Attili et al. (Br. J. Dermatol. 161(1), 170-173. 2009) published a clinical open pilot study of ambulatory photodynamic therapy (PDT) using a wearable low-irradiance OLEDs in the treatment of nonmelanoma skin cancer, suggesting that OLED-PDT is less painful than conventional PDT with the added advantage of being lightweight, and therefore has the potential for more convenient PDT at home.

Samuel et al. disclosed in EP 1444008B15 an ambulatory device for the use in a therapeutic and/or cosmetic treatment, the device comprises an OLEDs and poly(p-phenylene vinylene) (PPV) used as an example.

EP 1444008 discloses the Devices for the treatment of photodynamic therapy comprising OLEDs.

Organic light emitting diodes have many advantages over their inorganic counterpart (light emitting diodes—LEDs) in that they are intrinsically flexible, and can be coated on large area by, for example, printing tech-nologies, such as ink jet printing and screen printing.

However, in OLEDs active metals, such as Ba and Ca, are used as cathode. Therefore, OLEDs require excellent encapsulation to ensure long lifetime both in storage and in operation. Overall the production of OLEDs, a multilayer structure with each of several tons of nanometers, is still an elaborate and cost intensive task.

The use of organic light emitting electrochemical cells (OLECs, LEECs or LECs) for the therapy, prophylasis and/or diagnosis of diseases and/or cosmetic conditions is for different reasons advantageous as compared to the use of OLEDs.

First of all OLECs are very simple in their structure and therefore can be prepared easily. The preparation of devices is in the case of OLECs less complex as compared to the preparation of such surfaces in OLEDs. This is due to the fact 1) OLECs have a lower number of layers compared to OLEDs; 2) the emissive layer of OLEC can be as thick as several or several tens of micrometers, which allows the use of many available coating technologies, for example inkjeting printing, screen-printing and spray coating, in mass-production; 3) the requirements relating to homogeneity of the layer is less stringent. Thus, the production costs in particular for mass production may be much lower as compared to the ones of OLEDs.

Furthermore, OLECs do not rely on air-sensitive charge-injection layers or active metals such as Ba or Cs as cathode for electron injection, which further simplifies their preparation and makes them more cost efficient, as compared to OLEDs. This may also lead to the less stringent requirements for encapsulation of OLECs.

The underlying technology of OLECs differs from the ones of OLEDs or LEDs. Both OLEDs and LEDs are diodes with forward bias and reverse bias. In contrast to OLECs the I-V (current-voltage) curves of both OLEDs and LEDs are asymmetric. They represent semiconductor technologies whereas an OLEC is basically an electrochemical or more precisely an electrolytic cell. Charge transport in OLEDs occurs via the movement of holes and electrons from molecule to molecule until holes and electrons form so called excitons, i.e. electron-hole-pairs. Light is emitted when the exciton radiatively decays. In OLECs, upon applying a voltage, the electrolyte is oxidized at the anode and reduced at the cathode.

The molecular cations and anions diffuse under the electrical field and in the meanwhile doping the organic emissive materials until they meet together to form a so called p-n junction. Further an exciton is formed on the organic emissive compounds in the p-n junction. The radiative decay of the exciton leads to the emission of light. The original work and the principle of OLECs can be referred to the paper by Qibing Pei et al., Science, 1995, 269, 1086-1088. The OLECs can show symmetric I-V curves, have low driving voltages, and there is no need for active metals as cathode.

However, one drawback of OLEDs and OLECs is the broad emission due to the nature of organic emitters, which may lead to energy lost or to unwanted side effects. The broad emission spectrum of OLEDs and OLECs is not only unwanted in phototherapeutical applications but also in other technical field such as display and lighting applications. For example, for display application, organic emitters usually have a low color purity.

Another drawback of organic emitters, both in OLED and OLEC, is the limited quantum efficiency. According to quantum statistics, three triplets per singlets are formed in the OLED and OLEC if the probability of exciton formation is not spin-dependent. The probability of singlet exciton formation for devices based on fluorescent materials is only 25%. Hence, a funda-mental limit of an internal quantum efficiency of 25% is put on OLED/OLEC which are solely based on singlet emitters. This limit can be overcome by incorporating phosphorescent dopants, also called triplet emitters, usually complexes containing a heavy metal, which can increase spin-orbital coupling and harvest both singlet and triplet excitons. However, the metal complex is difficult to synthesize and it has stability problems. So far, a stable (deep) blue triplet emitter is still elusive. Moreover, because the triplet level of the organic materials is typically at least 0.5 eV higher than singlet level, a blue triplet emitter having itself a big band-gap (or HOMO-LUMO gap) will put extremely hard requirements on host materials and the charge transport materials in the adjacent layers.

On the other hand, another class of emissive material, so-called quantum dot or mono-dispersive nanocrystal as described below, has also drawn considerable attention in the last years. The advantages of quantum dot are: 1) theoretical internal quantum efficiency as high as 100%, compared to 25% of the singlet organic emitter; 2) soluble in common organic solvents; 3) emission wavelength can be easily tuned by the core size; 4) narrow emission spectrum; 5) intrinsic stability in inorganic materials.

The first mono-dispersive nanocrystals including a semiconducting material, also referred to herein as quantum dots or QDs, were based on CdE (E=S, Se, Te) and were produced using the so called TOPO (trioctyl phosphine oxide) method by Bawendi and later modified by Katari et al. A review on synthesis of QDs is given by Murray, Norris and Bawendi, “Synthesis and characterization of nearly monodisperse CdE (E=sulfur, selen, tellurium) semiconductor nanocrystallites”, J. Am. Chem. Soc. 115[19], 8706-8715, 1993. The mostly-reported caps of quantum dots are based on trioctylphosphine oxide (TOPO) or trioctylphosphine (TOP), which are supposed to have electron transporting properties.

The first light-emitting diode based on CdSe QDs was reported by Alivisatos et al., “Light emitting diodes made from cadmium selenide nanocrystals and a semiconducting polymer”, Nature (London) 370[6488], 354-357, 1994, where a multilayer consisting of QDs was sandwiched between PPV (poly(p-phenylene-vinylene)) and an electrode, giving emission in red upon applying voltage. Bulovic et al., “Electroluminescence from single monolayers of nanocrystals in molecular organic devices. Nature (London) 420[6917], 800-803, 2002 describe use of a single monolayer of CdSe QDs sandwiched between two organic layers.

But one major problem of known QD LEDs is the huge energy level offset between the QDs and the adjacent organic layers, for example CdSe QDs have a HOMO of −6.6 eV and LUMO of −4.4 eV (WO 2003/084292, WO 2007/095173), and on the other side functional organic materials have usually a LUMO>−3.0 eV and HOMO>−5.6 eV. The big energy offset prevents that QDs are efficiently electronically active in electroluminescent devices or other electronic devices.

Therefore the object of the present invention is to provide an improved electronic device.

So far, Leger et al, (Abstract of the 64^{th} Northwest Regional Meeting of the American Chemical Society, Tacoma, Wash., United States, June 28 to Jul. 1, 2009) disclosed a light emitting electrochemical cell comprising conjugated polymer and quantum dots with promising results. However, though conjugated polymers can easily be coated from solution, the performance of polymer OLEDs/OLECs is far behind that of OLEDs based on evaporated small molecule (SM) OLEDs. Furthermore, conjugated polymers have, due to the extended conjugation, in general a quite low triplet level. No conjugated polymer matrix for green triplet OLEDs has been reported or disclosed so far.

One objective of the present invention is, therefore, to provide a thin light source whose emission wavelengths can precisely be tailored. Thus, color purity of the emission should be improved. Another objective of the present invention is to provide a light emitting devices with high efficiency and less energy loss for display and lighting applications, especially in the ultraviolet (UV) and/or infrared (IR) region of the spectrum. Yet another objective of the present invention is the application of the light sources of the present invention in different technical fields such as display, general lighting, backlit applications, phototherapy and/or PDT. The light sources can easily produced and are particularly with regard to phototherapeutical applications user friendly which is mainly due to their size, potential device flexibility, and adaptable size, shape, irradiation wavelength and intensity of the irradiation.

Surprisingly it has been found that quantum dots can be used in OLECs in connection with organic functional materials such as emitters, host materials, hole transport materials, hole injection materials, electron transport materials, and electron injection materials in order to achieve the above mentioned objectives. Quantum dots can easily be produced and have a narrow emission spectrum in contrast to organic fluorescent or phosphorescent compounds. They can be tailored in terms of size which determines the quantum dot's emission maximum. High photoluminescent efficiency can also be obtained with quantum dots. Furthermore their emission intensity can be tailored by their concentration employed. Moreover, quantum dots are soluble in many solvents or can easily be made soluble in common organic solvents, allowing versatile processing methods, particularly printing methods such as screen printing, off-set printing, and ink jet printing.

The present invention relates to a light emitting electrochemical cell (QD-LEC) comprising at least one quantum dot, at least one ionic compound and at least one small organic functional materials selected from host materials, fluorescent emitters, phosphorescent emitters, hole transport materials (HTMs), hole injection materials (HIMs), electron transport materials (ETMs), and electron injection materials (EIMs).

In general, a quantum dot is a semiconductor whose excitons are confined in all three spatial dimensions. As a result, they have properties that are between those of bulk semiconductors and those of discrete molecules. There are several ways to prepare quantum dot structures, for example by chemical methods or by ion implantation, or in nanodevices made by state-of-the-art lithographic techniques.

The quantum dots of the present invention refer to colloidal semiconductor nanocrystals, also known as colloidal quantum dots, or nanodots or nanocrystals, which are produced by chemical methods.

The first mono-dispersive colloidal quantum dots including a semiconducting material were based on CdE (E=S, Se, Te) and were produced using the so called TOPO (trioctyl phosphine oxide) method by Bawendi and later modified by Katari et al. A review on synthesis of QDs is given by Murray, Norris and Bawendi, “Synthesis and characterization of nearly monodisperse CdE (E=sulfur, selen, tellurium) semiconductor nanocrystallites”, J. Am. Chem. Soc. 115[19], 8706-8715, 1993. While any method known to the skilled person can be used to create QDs, preferably a solution-phase colloidal method for controlled growth of inorganic QDs is used. The said colloidal methods are disclosed, e.g., by Alivisatos, A. P., “Semiconductor clusters, nanocrystals, and quantum dots,” Science 271:933 (1996); X. Peng, M. Schlamp, A. Kadavanich, A. P. Alivisatos, “Epitaxial growth of highly luminescent CdSe/CdS Core/Shell nanocrystals with photostability and electronic accessibility,” J. Am. Chem. Soc. 30:7019-7029 (1997); and C. B. Murray, D. J. Norris, M. G. Bawendi, “Synthesis and characterization of nearly monodisperse CdE (E=sulfur, selenium, tellurium) semiconductor nanocrystallites,” J. Am. Chem. Soc. 115:8706 (1993). These methods allow low cost processability without the need for clean rooms and expensive manufacturing equipment. In these methods, metal precursors that undergo pyrolysis at high temperature are rapidly injected into a hot solution of organic surfactant molecules. These precursors break apart at high temperatures and react to nucleate nanocrystals. After this initial nucleation phase, a growth phase begins by the addition of monomers to the growing crystal. Thus, crystalline nanoparticles are obtained in solution that has an organic surfactant molecule coating their surface.

In these methods, synthesis occurs as an initial nucleation event that takes place over seconds, followed by crystal growth at elevated temperature for several minutes. Parameters such as the temperature, types of surfactants present, precursor materials, and ratios of surfactants to monomers can be modified so as to change the nature and progress of the reaction. The temperature controls the structural phase of the nucleation event, rate of decomposition of precursors, and rate of growth. The organic surfactant molecules mediate both solubility and control of the nanocrystal shape. The ratio of surfactants to monomer, surfactants to each other, monomers to each other, and the individual concentrations of monomers strongly influence the kinetics of growth.

The QD-LECs according to the present invention can comprise as many quantum dots as required to achieve the desired effect. Preferably the QD-LECs comprise less than 100, particularly preferably less than 70 and very particularly preferably less than 40 different quantum dots. In a further preferred embodiment the said array comprises less than 20 different types of quantum dots.

In yet another embodiment the QD-LECs according to the present invention comprise 4, preferably 3, particularly preferably 2, and very particularly preferably 1 quantum dot(s).

Preference is given to QD-LECs comprising one quantum dot.

QD-LECs according to the present invention preferably comprise the quantum dot(s) in each a concentration of at least 0.1 wt %, particularly preferably at least 0.5 wt %, and very particularly preferably of at least 3 wt % with respect to the total amount of the emissive layer.

In one embodiment the QD-LECs according to the present invention comprise less than 15, preferably less than 10, particularly preferably less than 7, and very particularly preferably less than 5 small organic functional material(s).

The small organic functional materials according to the present invention are materials which are commonly used in the field of organic electronics and which are well known to one skilled in the art. A preferred compilation of small organic functional materials is disclosed in EP 09015222.4 and EP 10002558.4.

The term small organic functional materials refers to small molecules having the desired host, light emitting, hole injecting, hole transporting, electron injecting, and/or electron transporting properties.

A small molecule according to the present invention is a molecule which is not a polymer, oligomer, dendrimer, or a blend. In particular, repeating structures are absent in small molecules. The molecular weight of small molecules is typically in the range of polymers with a low number of repeating units, oligomers or less. The molecular weight of the small molecule may be preferably below 4000 g/mol, particularly preferably below 3000 g/mol, and very particularly preferably below 2000 g/mol.

Polymers may have 10 to 10000, particularly preferably 20 to 5000 and very particularly preferably 50 to 2000 repeating units. Oligomers may have 2 to 9 repeating units. The branching index of the polymers and oligomers is between 0 (linear polymer without branching) and 1 (completely branched dendrimer). The term dendrimer as used herein is defined according to M. Fischer et al. in Angew. Chem., Int. Ed. 1999, 38, 885.

The molecular weight (M_{W}) of the polymers may preferably be in the range of about 10000 to about 2000000 g/mol, particularly preferably in the range of about 100000 to about 1500000 g/mol, and very particularly preferably in the range of about 200000 to about 1000000 g/mol. The determination of M_{W} can be performed according to standard techniques known to the person skilled in the art by employing gel permeation chromatography (GPC) with polystyrene as internal standard, for instance.

A blend may be a mixture including at least one polymeric dendrimeric, or oligomeric component.

The term host, or matrix material refers to a material having a bigger energy gap as emitter, and have either electron or hole transport properties or both. In the case of singlet OLEDs, it is highly desired that the absorption spectrum of emitter overlaps essentially with photoluminescent spectrum of the host to ensure energy transfer. The QD-LECs according to the present invention may comprise at least one small molecular host. In principle any small molecule host or matrix material can be used according to the present invention

The term emitter refers to a material which upon receiving excitonic energy optically or electronically undergoes radiative decay to emit light. Principally, there are two classes of emitters, fluorescent emitters and phosphorescent emitters. The term fluorescent emitter relates to materials or compounds which undergo a radiative transition from an excited singlet state to its ground state. Thus, fluorescent emitters are sometimes also called singlet emitters. The term phosphorescent emitter relates to luminescent materials or compounds which include transition metals, which also comprise rare earth metals, lanthanides and actinides. Phosphorescent emitters predominately emit light by spin forbidden transitions occur, e.g., transitions from excited triplet and/or quintet states. However, a certain fraction of light emitted by phosphorescent emitters may also be caused by light emitting transitions from singlet states.

The term dopant as employed herein is also used for the term emitter or emitter material. In principle any small molecule light emitting compound can be used according to the present invention.

The QD-LECs according to the present invention may comprise at least one small organic functional material selected from hole transport materials (HTM). A HTM is characterized in that it is a material or unit capable of transporting holes (i.e. positive charges) injected from a hole injecting material or an anode.

The QD-LECs according to the present invention comprise 4, preferably 3, particularly preferably 2, and very particularly preferably 1 HTM(s). Preference is given to QD-LECs comprising one HTM.

QD-LECs according to the present invention preferably comprise the HTM(s) in each a concentration of at least 0.1 wt %, particularly preferably at least 2 wt %, and very particularly preferably of at least 10 wt % with respect to the total amount of the hole transport layer.

The QD-LECs according to the present invention may comprise at least one small organic functional material selected from hole injection materials (HIM). A HIM refers to a material or unit capable of facilitating holes (i.e. positive charges) injected from an anode.

The QD-LECs according to the present invention comprise 4, preferably 3, particularly preferably 2, and very particularly preferably 1 HIM(s). Preference is given to QD-LECs comprising one HIM.

QD-LECs according to the present invention preferably comprise the HIM(s) in each a concentration of at least 0.1 wt %, particularly preferably at least 0.5 wt %, and very particularly preferably of at least 3 wt % with respect to the total amount of hole injection layer.

The QD-LECs according to the present invention may comprise at least one small organic functional material selected from electron transport materials (ETM). An ETM refers to a material capable of transporting electrons (i.e. negative charges) injected from an EIM or a cathode.

The QD-LECs according to the present invention comprise 4, preferably 3, particularly preferably 2, and very particularly preferably 1 ETM(s). Preference is given to QD-LECs comprising one ETM.

QD-LECs according to the present invention preferably comprise the ETM(s) in each a concentration of at least 0.1 wt %, particularly preferably at least 2 wt %, and very particularly preferably of at least 10 wt % with respect to the total amount of the electron transporting layer.

The QD-LECs according to the present invention may comprise at least one small organic functional material selected from electron injection materials (EIM). An EIM refers to a material capable of facilitating electrons (i.e. negative charges) injected from cathode into an organic layer.

The QD-LECs according to the present invention comprise 4, preferably 3, particularly preferably 2, and very particularly preferably 1 EIM(s). Preference is given to QD-LECs comprising one EIM.

QD-LECs according to the present invention preferably comprise the EIM(s) in each a concentration of at least 0.1 wt %, particularly preferably at least 0.5 wt %, and very particularly preferably of at least 3 wt % with respect to the total amount of the electron injection layer.

In principle any small molecule EIM known to one skilled in the art can be employed according to the present invention. Further to EIM mentioned elsewhere herein, suitable EIMs comprise at least one organic compound selected from metal complexes of 8-hydroxyquinoline, heterocyclic organic compounds, fluorenones, fluorenylidene methane, perylenetetracarboxylic acid, anthraquinone dimethanes, diphenoquinones, anthrones, anthraquinonediethylene-diamines, isomers and derivates thereof can be used according to the invention.

Metal complexes of 8 hydroxyquinoline, such as, for example, Alq₃ and Gaq₃, can be used as EIM. A reducing doping with alkali metals or alkaline-earth metals, such as, for example, Li, Cs, Ca or Mg, at the interface to the cathode is advantageous. Preference is given to combinations which include Cs, for example Cs and Na, Cs and K, Cs and Rb or Cs, Na and K.

Heterocyclic organic compounds, such as, for example, 1,10-phenanthroline derivatives, benzimidazoles, thiopyran dioxides, oxazoles, triazoles, imidazoles or oxadiazoles, are likewise suitable. Examples of suitable five-membered rings containing nitrogen are oxazoles, thiazoles, oxadiazoles, thiadiazoles, triazoles, and compounds which are disclosed in US 2008/0102311 A1.

Preferred EIMs are selected from compounds with the formulae (1) to (3), which may be substituted or unsubstituted.

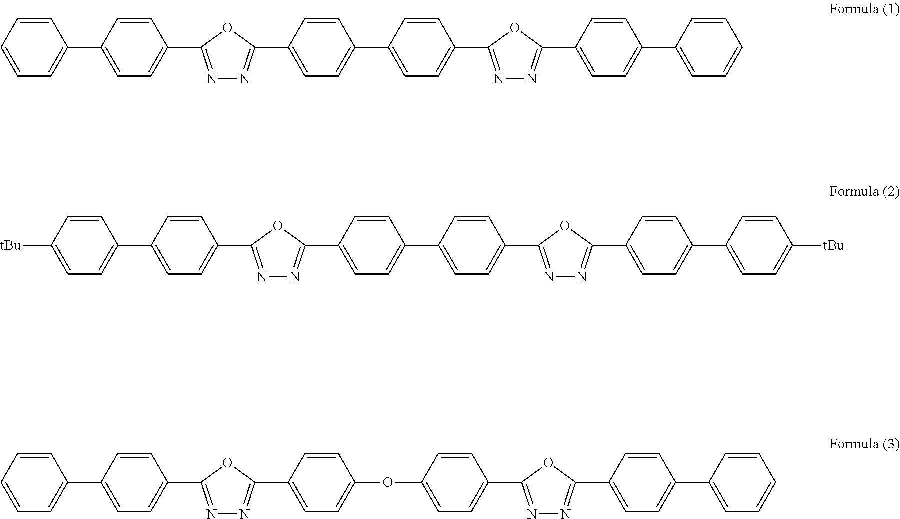 [SMILES: c1ccc(-c2ccc(-c3nnc(-c4ccc(-c5ccc(-c6nnc(-c7ccc(-c8ccccc8)cc7)o6)cc5)cc4)o3)cc2)cc1 ; CC(C)(C)c1ccc(-c2ccc(-c3nnc(-c4ccc(-c5ccc(-c6nnc(-c7ccc(-c8ccc(C(C)(C)C)cc8)cc7)o6)cc5)cc4)o3)cc2)cc1 ; c1ccc(-c2ccc(-c3nnc(-c4ccc(Oc5ccc(-c6nnc(-c7ccc(-c8ccccc8)cc7)o6)cc5)cc4)o3)cc2)cc1]

Organic compounds, such as fluorenones, fluorenylidene methane, perylenetetracarboxylic acid, anthraquinone dimethanes, diphenoquinones, anthrones and anthraquinonediethylenediamines, can also be employed, for example

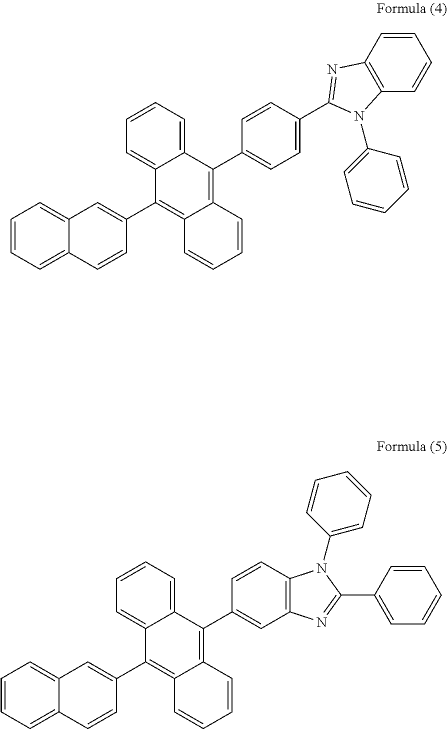 [SMILES: c1ccc(-n2c(-c3ccc(-c4c5ccccc5c(-c5ccc6ccccc6c5)c5ccccc45)cc3)nc3ccccc32)cc1 ; c1ccc(-c2nc3cc(-c4c5ccccc5c(-c5ccc6ccccc6c5)c5ccccc45)ccc3n2-c2ccccc2)cc1]

In principle any ETM known to one skilled in the art can be employed according to the present invention. Further to ETM mentioned elsewhere herein, suitable ETMs are selected from the group consisting of imidazoles, pyridines, pyrimidines, pyridazines, pyrazines, oxadiazoles, chinolines, chinoxalines, anthracenes, benzanthracenes, pyrenes, perylenes, benzimidazoles, triazines, ketones, phosphinoxides, phenazines, phenanthrolines, triarylboranes, isomers and derivatives thereof.

Further suitable ETMs are selected from imidazoles, pyridines, pyrimidines, pyridazines, pyrazines, oxadiazoles, chinolines, chinoxalines, anthracenes, benzanthracenes, pyrenes, perylenes, benzimidazoles, triazines, ketones, phosphinoxides, phenazines, phenanthrolines, and triarylboranes.

Further suitable ETMs for electron-transporting layers are metal chelates of 8 hydroxyquinoline (for example Liq, Alq₃, Gaq₃, Mgq₂, Znq₂, Inq₃, Zrq₄), Balq, 4 azaphenanthrene-5-ol/Be complexes (U.S. Pat. No. 5,529,853 A; e.g. formula (6)), butadiene derivatives (U.S. Pat. No. 4,356,429), heterocyclic optical brighteners (U.S. Pat. No. 4,539,507), benzazoles, such as, for example, 1,3,5-tris(2-N-phenylbenzimidazolyl)benzene (TPBI) (U.S. Pat. No. 5,766,779, formula (7)), 1,3,5-triazines, pyrenes, anthracenes, tetracenes, fluorenes, spirobifluorenes, dendrimers, tetracenes, for example rubrene derivatives, 1,10-phenanthroline derivatives (JP 2003/115387, JP 2004/311184, JP 2001/267080, WO 2002/043449), silacyl-cyclopentadiene derivatives (EP 1480280, EP 1478032, EP 1469533), pyridine derivatives (JP 2004/200162 Kodak), phenanthrolines, for example BCP and Bphen, also a number of phenanthrolines bonded via biphenyl or other aromatic groups (US 2007/0252517 A1) or phenanthrolines bonded to anthracene (US 2007/0122656 A1, e.g. formulae (8) and (9)), 1,3,4-oxadiazoles, for example formula (10), triazoles, for example formula (II), triarylboranes, for example also with Si, benzimidazole derivatives and other N heterocyclic compounds (cf. US 2007/0273272 A1), silacyclopentadiene derivatives, borane derivatives, Ga oxinoid complexes.

Preference is given to 2,9,10-substituted anthracenes (with 1- or 2-naphthyl and 4- or 3-biphenyl) or molecules which contain two anthracene units (US 2008/0193796 A1).

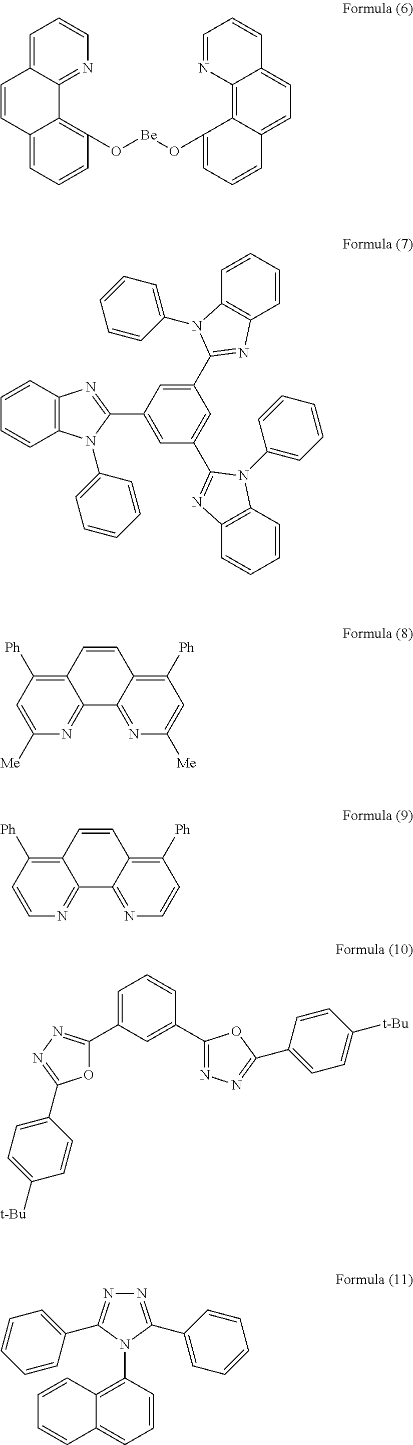

Preference is likewise given to anthracene-benzimidazole derivatives, such as, for example, the compounds of formulae (12) to (14), and as disclosed in, e.g., U.S. Pat. No. 6,878,469 B2, US 2006/147747 A, and EP 1551206 A1.

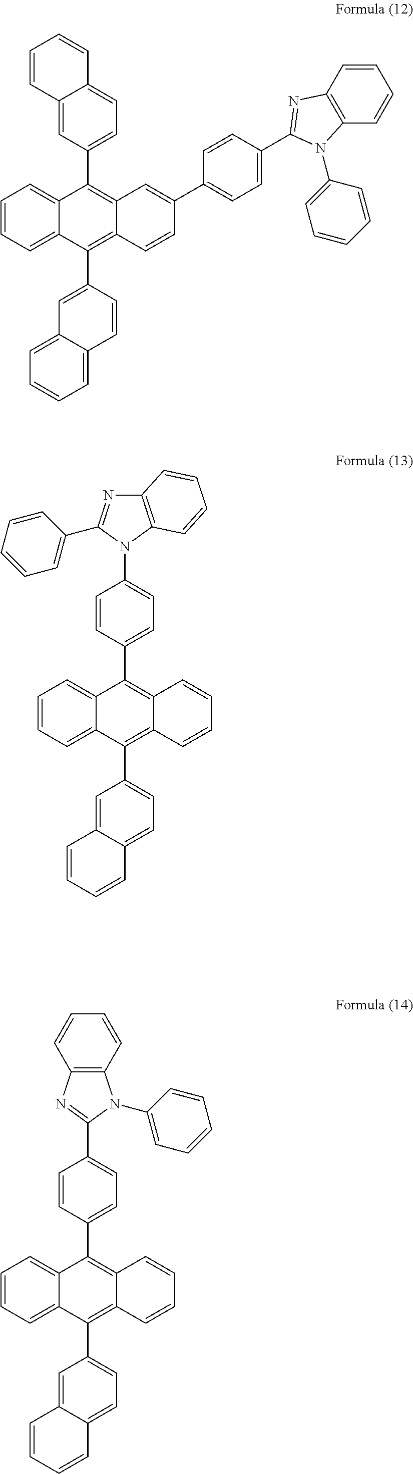 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2nc3ccccc3n2-c2ccc(-c3c4ccccc4c(-c4ccc5ccccc5c4)c4ccccc34)cc2)cc1 ; c1ccc(-n2c(-c3ccc(-c4c5ccccc5c(-c5ccc6ccccc6c5)c5ccccc45)cc3)nc3ccccc32)cc1]

Further to HIMs mentioned elsewhere herein, suitable HIMs are phenylenediamine derivatives (U.S. Pat. No. 3,615,404), arylamine derivatives (U.S. Pat. No. 3,567,450), amino-substituted chalcone derivatives (U.S. Pat. No. 3,526,501), styrylanthracene derivatives (JP Showa 54 (1979) 110837), hydrazone derivatives (U.S. Pat. No. 3,717,462), acylhydrazones, stilbene derivatives (JP Showa 61 (1986) 210363), silazane derivatives (U.S. Pat. No. 4,950,950), polysilane compounds (JP Heisei 2 (1990) 204996), PVK, porphyrin compounds (JP Showa 63 (1988) 2956965, U.S. Pat. No. 4,720,432), aromatic tertiary amines and styrylamines (U.S. Pat. No. 4,127,412), triphenylamines of the benzidine type, triphenylamines of the styrylamine type, and triphenylamines of the diamine type. Arylamine dendrimers can also be used (JP Heisei 8 (1996) 193191), as can phthalocyanine derivatives, naphthalocyanine derivatives, or butadiene derivatives, are also suitable.

Preferably, the HIM is selected from monomeric organic compound comprising amine, triarylamine, thiophene, carbazole, phthalocyanine, porphyrine and their derivatives.

Particular preference is given to the tertiary aromatic amines (US 2008/0102311 A1), for example N,N′-diphenyl-N,N′-di(3-tolyl)benzidine (=4,4′-bis[N-3-methylphenyl]-N-phenylamino)biphenyl (NPD) (U.S. Pat. No. 5,061,569), N,N′-bis(N,N′-diphenyl-4-aminophenyl)-N,N-diphenyl-4,4′-diamino-1,1′-biphenyl (TPD 232) and 4,4′,4″-tris[3-methylphenyl)phenylamino]-triphenylamine (MTDATA) (JP Heisei 4 (1992) 308688) or phthalocyanine derivatives (for example H2Pc, CuPc, CoPc, NiPc, ZnPc, Pd Pc, FePc, MnPc, ClAlPc, CIGaPc, ClInPc, ClSnPc, Cl₂SiPc, (HO)AlPc, (HO)GaPc, VOPc, TiOPc, MoOPc, GaPc-O—GaPc).

Particular preference is given to the following triarylamine compounds of the formulae (15) (TPD 232), (16), (17), and (18), which may also be substituted, and further compounds as disclosed in U.S. Pat. No. 7,399,537 B2, US 2006/0061265 A1, EP 1661888 A1, and JP 08292586A. Further compounds suitable as hole injection material are disclosed in EP 0891121 A1 and EP 1029909 A1. Hole injection layers in general are described in US 2004/0174116.

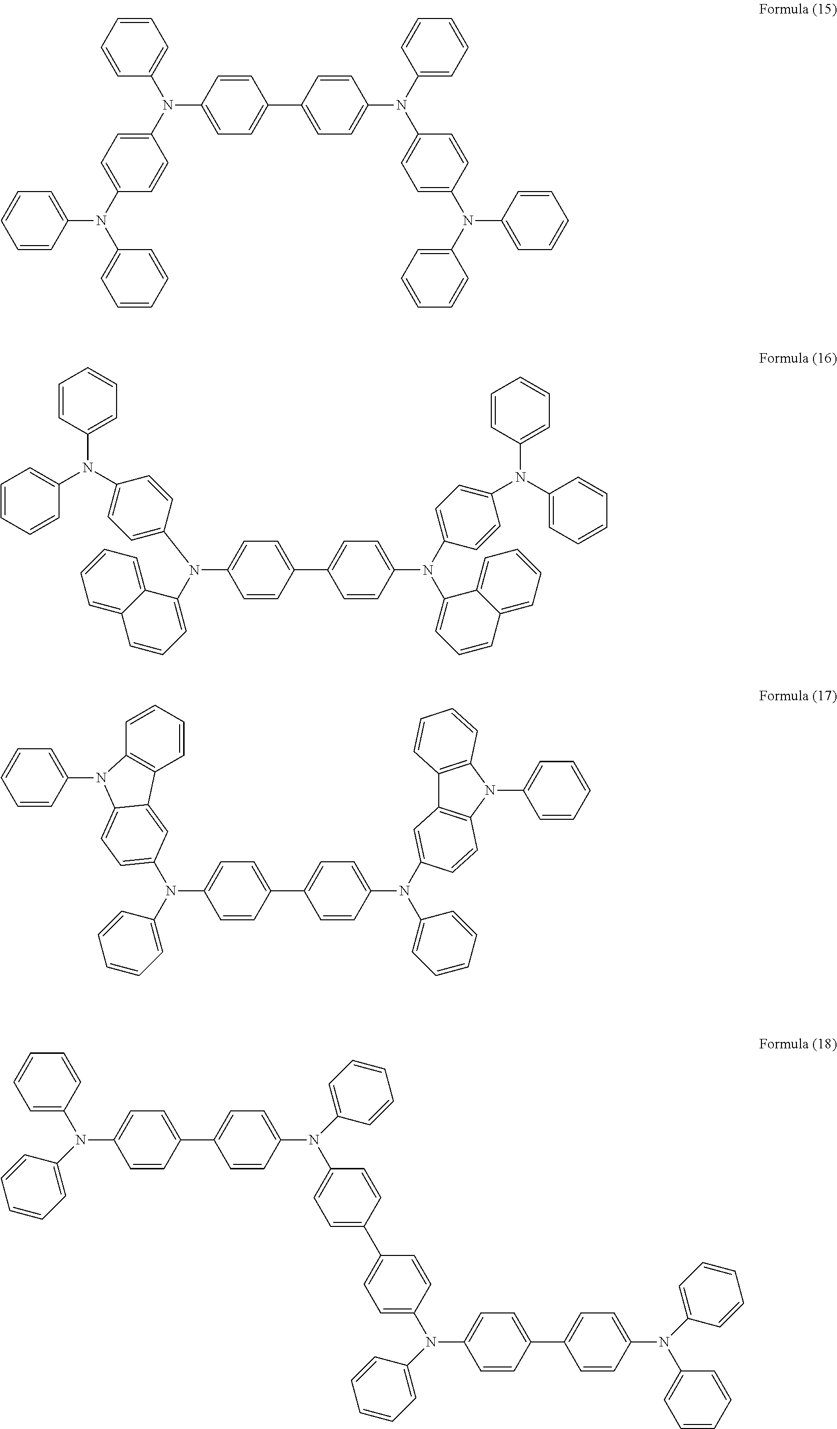 [SMILES: c1ccc(N(c2ccccc2)c2ccc(N(c3ccccc3)c3ccc(-c4ccc(N(c5ccccc5)c5ccc(N(c6ccccc6)c6ccccc6)cc5)cc4)cc3)cc2)cc1 ; c1ccc(N(c2ccccc2)c2ccc(N(c3ccc(-c4ccc(N(c5ccc(N(c6ccccc6)c6ccccc6)cc5)c5cccc6ccccc56)cc4)cc3)c3cccc4ccccc34)cc2)cc1 ; c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1]

In principle any HTM known to one skilled in the art can be employed in formulations according to the present invention. Further to HTM mentioned elsewhere herein, HTM is preferably selected from amines, triarylamines, thiophenes, carbazoles, phthalocyanines, porphyrines, isomers and derivatives thereof. HTM is particularly preferably selected from amines, triarylamines, thiophenes, carbazoles, phthalocyanines, and porphyrines.

Suitable small molecule materials for hole-transporting are phenylenediamine derivatives (U.S. Pat. No. 3,615,404), arylamine derivatives (U.S. Pat. No. 3,567,450), amino-substituted chalcone derivatives (U.S. Pat. No. 3,526,501), styrylanthracene derivatives (JP A 56-46234), polycyclic aromatic compounds (EP 1009041), polyarylalkane derivatives (U.S. Pat. No. 3,615,402), fluorenone derivatives (JP A 54-110837), hydrazone derivatives (U.S. Pat. No. 3,717,462), stilbene derivatives (JP A 61-210363), silazane derivatives (U.S. Pat. No. 4,950,950), polysilanes (JP A 2-204996), aniline copolymers (JP A 2-282263), thiophene oligomers, polythiophenes, PVK, polypyrroles, polyanilines and further copolymers, porphyrin compounds (JP A 63-2956965), aromatic dimethylidene-type compounds, carbazole compounds, such as, for example, CDBP, CBP, mCP, aromatic tertiary amine and styrylamine compounds (U.S. Pat. No. 4,127,412), and monomeric triarylamines (U.S. Pat. No. 3,180,730).

Preference is given to aromatic tertiary amines containing at least two tertiary amine units (U.S. Pat. No. 4,720,432 and U.S. Pat. No. 5,061,569), such as, for example, 4,4′-bis[N-(1-naphthyl)-N-phenylamino]biphenyl (NPD) (U.S. Pat. No. 5,061,569) or MTDATA (JP A 4-308688), N,N,N′,N′-tetra(4-biphenyl)diaminobiphenylene (TBDB), 1,1-bis(4-di-p-tolylaminophenyl)cyclohexane (TAPC), 1,1-bis(4-di-p-tolylaminophenyl)-3-phenylpropane (TAPPP), 1,4-bis[2-[4-[N,N-di(p-tolyl)amino]phenyl]vinyl]benzene (BDTAPVB), N,N,N′,N′-tetra-p-tolyl-4,4′-diaminobiphenyl (TTB), TPD, N,N,N′,N′-tetraphenyl-4,4′″-diamino-1,1′:4′,″:4″,1′″-quaterphenyl, likewise tertiary amines containing carbazole units, such as, for example, 4 (9H-carbazol-9-yl)-N,N-bis[4-(9H-carbazol-9-yl)phenyl]benzeneamine (TCTA). Preference is likewise given to hexaazatriphenylene compounds in accordance with US 2007/0092755 A1.

Particular preference is given to the following triarylamine compounds of the formulae (19) to (24), which may also be substituted, and as disclosed in EP 1162193 A1, EP 650955 A1, Synth. Metals 1997, 91(1-3), 209, DE 19646119 A1, WO 2006/122630 A1, EP 1860097 A1, EP 1834945 A1, JP 08053397 A, U.S. Pat. No. 6,251,531 B1, and WO 2009/041635.

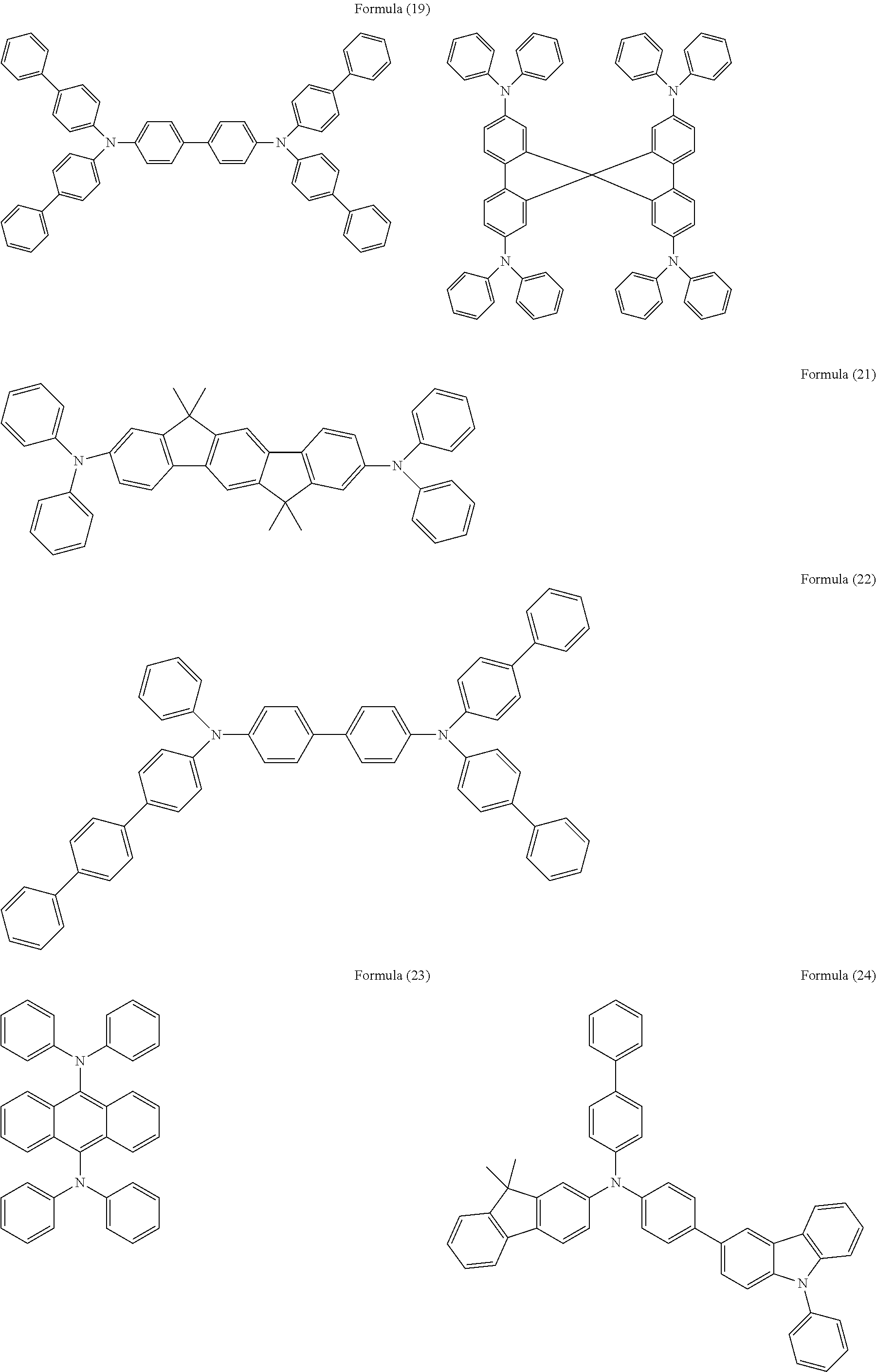

Preferred host materials suitable for fluorescent emitter are selected from anthracenes, benzanthracenes, indenofluorenes, fluorenes, spirobifluorenes, phenanthrenes, dehydrophenanthrenes, thiophenes, triazines, imidazole and derivatives thereof.

Preferred host materials suitable for fluorescent emitter are selected from anthracenes, benzanthracenes, indenofluorenes, fluorenes, spirobifluorenes, phenanthrenes, dehydrophenanthrenes, thiophenes, triazines, and imidazole.

The QD-LECs according to the present invention comprise 4, preferably 3, particularly preferably 2, and very particularly preferably 1 host material(s). Preference is given to QD-LECs comprising one host material. In case the QD-LECs comprises more than one host material the term co-host is often used for additional host materials.

Particularly preferred host materials for fluorescent emitter are selected from the classes of the oligoarylenes (for example 2,2′,7,7′-tetraphenyl-spirobifluorene in accordance with EP 676461 or dinaphthylanthracene), in particular the oligoarylenes containing condensed aromatic groups, such as, for example, phenanthrene, tetracene, coronene, chrysene, fluorene, spirofluorene, perylene, phthaloperylene, naphthaloperylene, decacyclene, rubrene, the oligoarylenevinylenes (for example 4,4′-bis(2,2-diphenyl-ethenyl)-1,1′-biphenyl (DPVBi) or 4,4-bis-2,2-diphenylvinyl-1,1-spirobi-phenyl (spiro-DPVBi) in accordance with EP 676461), the polypodal metal complexes (for example in accordance with WO 2004/081017), in particular metal complexes of 8 hydroxyquinoline, for example aluminium(III) tris(8-hydroxyquinoline) (aluminium quinolate, Alq₃) or bis(2-methyl-8-quinolinolato)-4-(phenylphenolinolato)aluminium, also with imidazole chelate (US 2007/0092753 A1) and quinoline-metal complexes, aminoquinoline-metal complexes, benzoquinoline-metal complexes, the hole-conducting compounds (for example in accordance with WO 2004/058911), the electron-conducting compounds, in particular ketones, phosphine oxides, sulfoxides, etc. (for example in accordance with WO 2005/084081 and WO 2005/084082), the atropisomers (for example in accordance with WO 2006/048268), the boronic acid derivatives (for example in accordance with WO 2006/117052) or the benzanthracenes (e.g. DE 102007024850). Particularly preferred host materials are selected from the classes of the oligoarylenes, containing naphthalene, anthracene, benzanthracene and/or pyrene, or atropisomers of these compounds, the ketones, the phosphine oxides and the sulfoxides. Very particularly preferred host materials are selected from the classes of the oligoarylenes, containing anthracene, benzanthracene and/or pyrene, or atropisomers of these compounds. For the purposes of this invention, an oligoarylene is intended to be taken to mean a compound in which at least three aryl or arylene groups are bonded to one another.

Further preferred host materials for fluorescent emitter are selected, in particular, from compounds of the formula (25)

Ar⁴—(Ar⁵)ₚ—Ar⁶ Formula (25)

wherein
Ar⁴, Ar⁵, Ar⁶ are on each occurrence, identically or differently, an aryl or heteroaryl group having 5 to 30 aromatic ring atoms, which may be substituted by one or more radicals and
p is 1, 2, or 3,
the sum of the π-electrons in Ar⁴, Ar⁵ and Ar⁶ is at least 30 if p=1 and is at least 36 if p=2 and is at least 42 if p=3.

It is particularly preferred in the host materials of the formula (25) for the group Ar⁵ to stand for anthracene, which may be substituted by one or more radicals R¹, and for the groups Ar⁴ and Ar⁶ to be bonded in the 9 and 10-positions. Very particularly preferably, at least one of the groups Ar⁴ and/or Ar⁶ is a condensed aryl group selected from 1- or 2-naphthyl, 2-, 3- or 9-phenanthrenyl or 2-, 3-, 4-, 5-, 6- or 7-benzanthracenyl, each of which may be substituted by one or more radicals R¹. Anthracene-based compounds are described in US 2007/0092753 A1 and US 2007/0252517 A1, for example 2-(4-methylphenyl)-9,10-di-(2-naphthyl)anthracene, 9-(2-naphthyl)-10-(1,1′-biphenyl)anthracene and 9,10-bis[4-(2,2-diphenyl-ethenyl)phenyl]anthracene, 9,10-diphenylanthracene, 9,10-bis(phenyl-ethynyl)anthracene and 1,4-bis(9′-ethynylanthracenyl)benzene. Preference is also given to host materials containing two anthracene units (US 2008/0193796 A1), for example 10,10′-bis[1,1′,4′,1″]terphenyl-2-yl-9,9′-bisanthracenyl.

Further preferred host materials are derivatives of arylamine, styrylamine, fluorescein, perynone, phthaloperynone, naphthaloperynone, diphenyl-butadiene, tetraphenylbutadiene, cyclopentadienes, tetraphenylcyclo-pentadiene, pentaphenylcyclopentadiene, coumarine, oxadiazole, bisbenzoxazoline, oxazone, pyridine, pyrazine, imine, benzothiazole, benzoxazole, benzimidazole (US 2007/0092753 A1), for example 2,2′,2″-(1,3,5-phenylene)tris[1-phenyl-1H-benzimidazole], aldazines, stilbene, styrylarylene derivatives, for example 9,10-bis[4-(2,2-diphenyl-ethenyl)phenyl]anthracene, and distyrylarylene derivatives (U.S. Pat. No. 5,121,029), diphenylethylene, vinylanthracene, diaminocarbazole, pyran, thiopyran, diketopyrrolopyrrole, polymethine, mellocyanine, acridone, quinacridone, cinnamic acid esters and fluorescent dyes.

Particular preference is given to derivatives of arylamine and styrylamine, for example 4,4′-bis[N-(1-naphthyl)-N-(2-naphthyl)amino]biphenyl (TNB).

Preferred compounds with oligoarylene as hosts for fluorescent emitter are compounds as disclosed in, e.g., US 2003/0027016 A1, U.S. Pat. No. 7,326,371 B2, US 2006/043858 A, U.S. Pat. No. 7,326,371 B2, US 2003/0027016 A1, WO 2007/114358, WO 2008/145239, JP 3148176 B2, EP 1009044, US 2004/018383, WO 2005/061656 A1, EP 0681019B1, WO 2004/013073A1, U.S. Pat. No. 5,077,142, WO 2007/065678, and US 2007/0205412 A1. Particularly preferred oligoarylene-based compounds are compounds having the formulae (26) to (32).

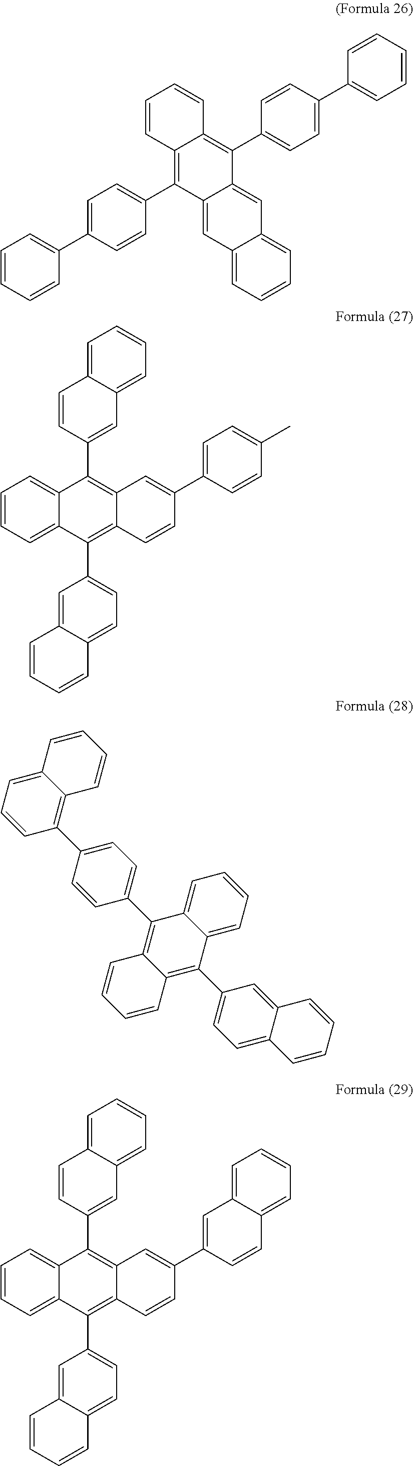

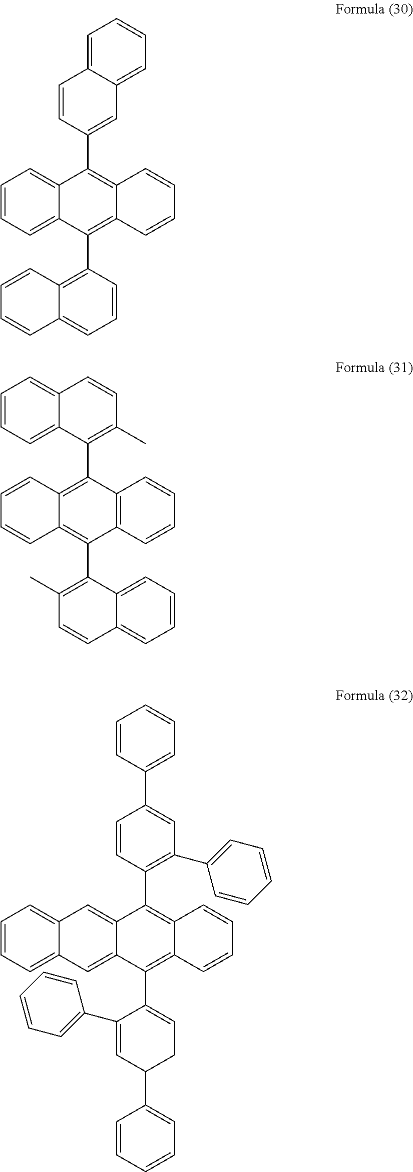

Further host materials for fluorescent emitter can be selected from spirobifluorene and derivates thereof, for example Spiro-DPVBi as disclosed in EP 0676461 and indenofluorene as disclosed in U.S. Pat. No. 6,562,485.

The preferred host materials for phosphorescent emitter, i.e. matrix materials, are selected from ketones, carbazoles, indolocarbazoles, triarylamines, indenofluorenes, fluorenes, spirobifluorenes, phenathrenes, dehydrophenanthrenes, thiophenes, triazines, imidazoles and their derivatives. Some preferred derivatives are described below in more details.

If a phosphorescent emitter is employed the host material must fulfil rather different characteristics as compared to host materials used for fluorescent emitter. The host materials used for phosphorescent emitter are required to have a triplet level which is higher in energy as compared to the triplet level of the emitter. The host material can either transport electrons or holes or both of them. In addition, the emitter is supposed to have large spin-orbital coupling constants in order to facilitate singlet-triplet mixing sufficiently. This can be enabled by using metal complexes.

Preferred matrix materials are N,N-biscarbazolylbiphenyl (CBP), carbazole derivatives (for example in accordance with WO 2005/039246, US 2005/0069729, JP 2004/288381, EP 1205527 or DE 102007002714), azacarbazoles (for example in accordance with EP 1617710, EP 1617711, EP 1731584, JP 2005/347160), ketones (for example in accordance with WO 2004/093207), phosphine oxides, sulfoxides and sulfones (for example in accordance with WO 2005/003253), oligophenylenes, aromatic amines (for example in accordance with US 2005/0069729), bipolar matrix materials (for example in accordance with WO 2007/137725), silanes (for example in accordance with WO 2005/111172), 9,9-diarylfluorene derivatives (e.g. in accordance with DE 102008017591), azaboroles or boronic esters (for example in accordance with WO 2006/117052), triazole derivatives, oxazoles and oxazole derivatives, imidazole derivatives, polyarylalkane derivatives, pyrazoline derivatives, pyrazolone derivatives, distyrylpyrazine derivatives, thiopyran dioxide derivatives, phenylene-diamine derivatives, tertiary aromatic amines, styrylamines, indoles, anthrone derivatives, fluorenone derivatives, fluorenylidenemethane derivatives, hydrazone derivatives, silazane derivatives, aromatic dimethyl-idene compounds, porphyrin compounds, carbodiimide derivatives, diphenylquinone derivatives, phthalocyanine derivatives, metal complexes of 8 hydroxyquinoline derivatives, such as, for example, Alq₃, the 8 hydroxyquinoline complexes may also contain triarylaminophenol ligands (US 2007/0134514 A1), various metal complex-polysilane compounds with metal phthalocyanine, benzoxazole or benzothiazole as ligand, hole-conducting polymers, such as, for example, poly(N-vinylcarbazole) (PVK), aniline copolymers, thiophene oligomers, polythiophenes, polythiophene derivatives, polyphenylene derivatives, polyfluorene derivatives.

Further particularly preferred matrix materials are selected from compounds comprising indolocarbazoles and their derivatives (e.g. formulae (33) to (39)), as disclosed for examples in DE 102009023155.2, EP 0906947B1, EP 0908787B1, EP 906948B1, WO 2008/056746A1, WO 2007/063754A1, WO 2008/146839A1, and WO 2008/149691A1.

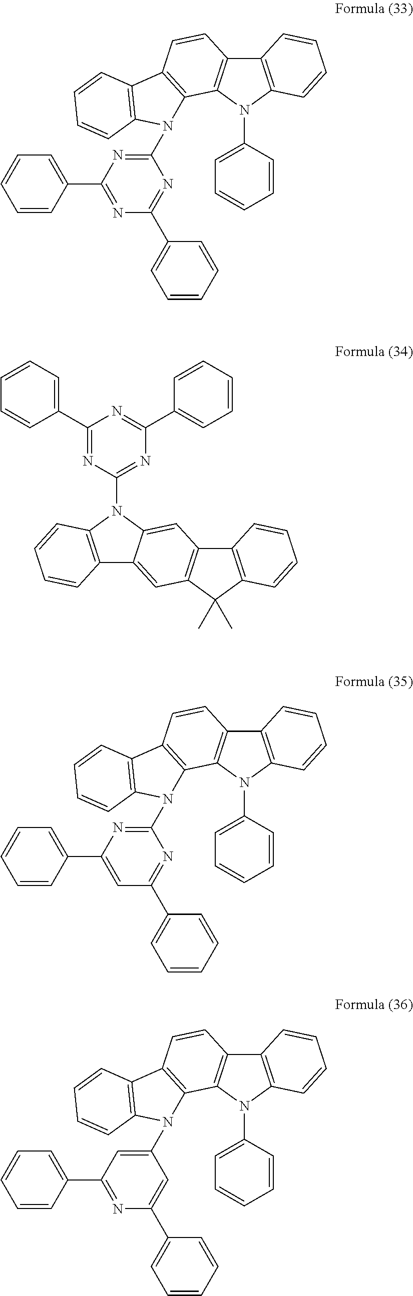 [SMILES: c1ccc(-c2nc(-c3ccccc3)nc(-n3c4ccccc4c4ccc5c6ccccc6n(-c6ccccc6)c5c43)n2)cc1 ; CC1(C)c2ccccc2-c2cc3c(cc21)c1ccccc1n3-c1nc(-c2ccccc2)nc(-c2ccccc2)n1 ; c1ccc(-c2cc(-c3ccccc3)nc(-n3c4ccccc4c4ccc5c6ccccc6n(-c6ccccc6)c5c43)n2)cc1 ; c1ccc(-c2cc(-n3c4ccccc4c4ccc5c6ccccc6n(-c6ccccc6)c5c43)cc(-c3ccccc3)n2)cc1]

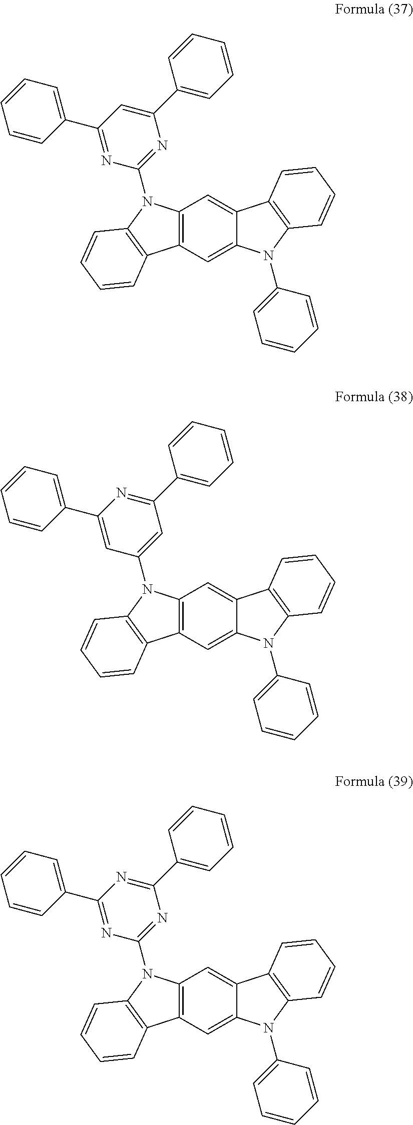 [SMILES: c1ccc(-c2cc(-c3ccccc3)nc(-n3c4ccccc4c4cc5c(cc43)c3ccccc3n5-c3ccccc3)n2)cc1 ; c1ccc(-c2cc(-n3c4ccccc4c4cc5c(cc43)c3ccccc3n5-c3ccccc3)cc(-c3ccccc3)n2)cc1 ; c1ccc(-c2nc(-c3ccccc3)nc(-n3c4ccccc4c4cc5c(cc43)c3ccccc3n5-c3ccccc3)n2)cc1]

Examples of preferred carbazole derivatives are, 1,3-N,N-dicarbazolebenzene (=9,9′-(1,3-phenylene)bis-9H-carbazole) (mCP), 9,9′-(2,2′-dimethyl[1,1′-biphenyl]-4,4′-diyl)bis-9H-carbazole (CDBP), 1,3-bis(N,N′-dicarbazole)benzene (=1,3-bis(carbazol-9-yl)benzene), PVK (polyvinylcarbazole), 3,5-di(9H-carbazol-9-yl)biphenyl and compounds of the formulae (40) to (44).

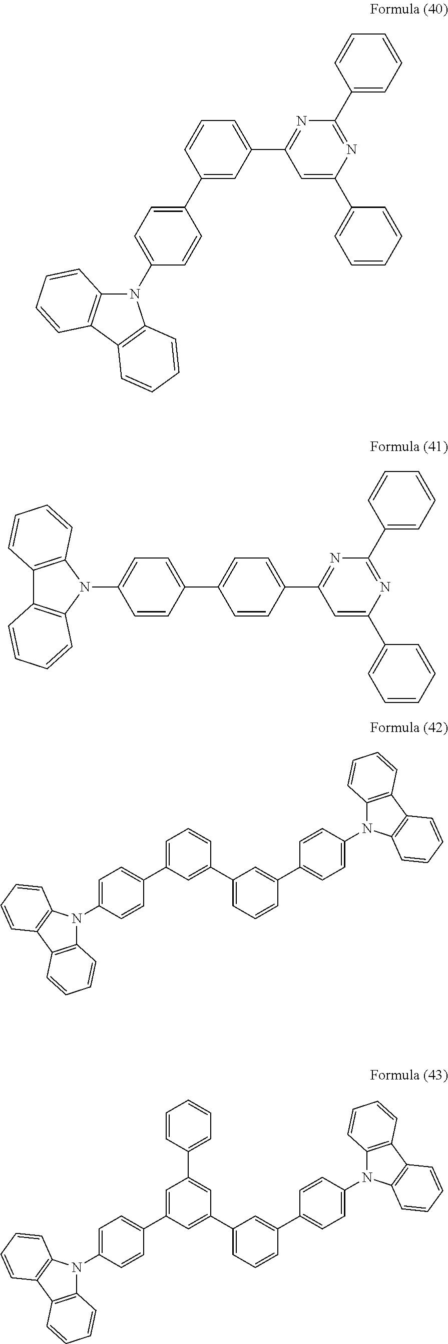 [SMILES: c1ccc(-c2cc(-c3cccc(-c4ccc(-n5c6ccccc6c6ccccc65)cc4)c3)nc(-c3ccccc3)n2)cc1 ; c1ccc(-c2cc(-c3ccc(-c4ccc(-n5c6ccccc6c6ccccc65)cc4)cc3)nc(-c3ccccc3)n2)cc1 ; c1cc(-c2ccc(-n3c4ccccc4c4ccccc43)cc2)cc(-c2cccc(-c3ccc(-n4c5ccccc5c5ccccc54)cc3)c2)c1 ; c1ccc(-c2cc(-c3ccc(-n4c5ccccc5c5ccccc54)cc3)cc(-c3cccc(-c4ccc(-n5c6ccccc6c6ccccc65)cc4)c3)c2)cc1]

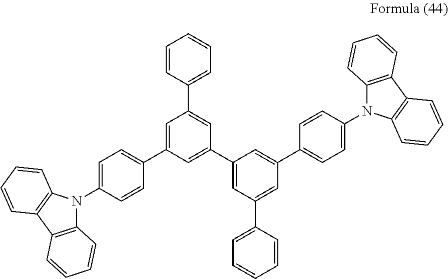 [SMILES: c1ccc(-c2cc(-c3ccc(-n4c5ccccc5c5ccccc54)cc3)cc(-c3cc(-c4ccccc4)cc(-c4ccc(-n5c6ccccc6c6ccccc65)cc4)c3)c2)cc1]

Preferred Si tetraaryl compounds are, for example, (US 2004/0209115, US 2004/0209116, US 2007/0087219 A1, US 2007/0087219 A1) the compounds of the formulae (45) to (59).

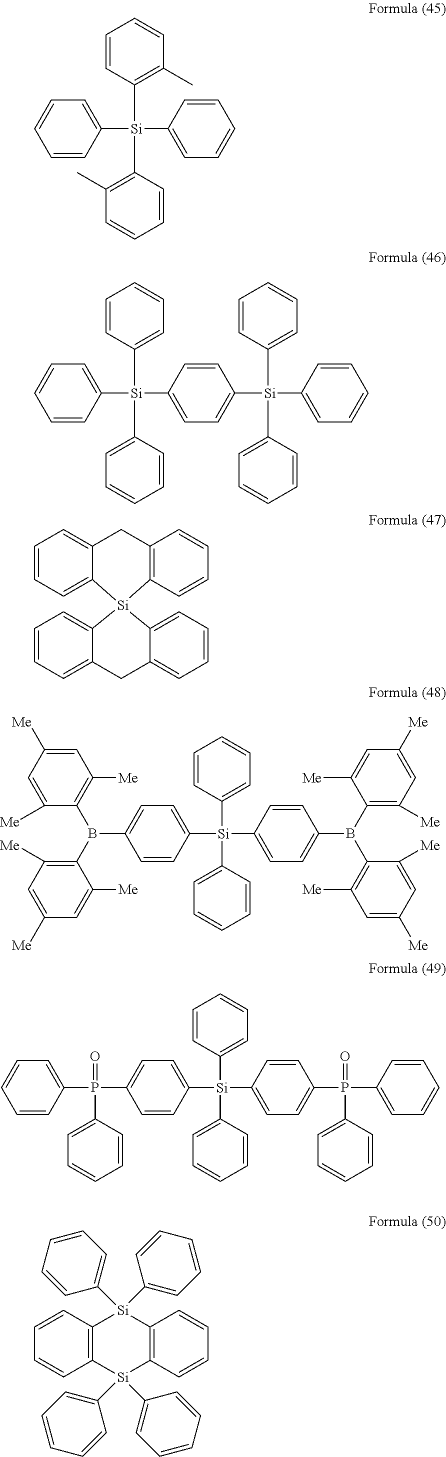

A particularly preferred matrix for phosphorescent dopants is the compound of formula (51) (EP 652273 B1)

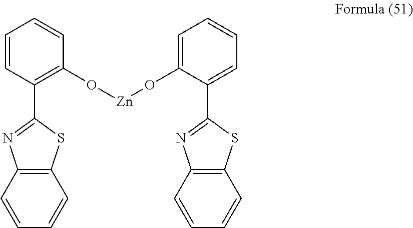

Further particularly preferred matrix materials for phosphorescent dopants are selected from compounds of the general formula (52) (EP 1923448A1).

[M(L)₂]ₙ Formula (52)

wherein M, L, and n are defined as in the reference. Preferably M is Zn, and L is quinolinate, and n is 2, 3 or 4. Very particularly preferred are [Znq₂]₂, [Znq₂]₃, and [Znq₂]₄.

Preference is given to co-hosts selected from metal oxinoid complexes whereby lithium quinolate (Liq) or Alq₃ are particularly preferred.

In a preferred embodiment the said QD-LEDs comprise at least one small molecule organic fluorescent emitter. Thus, the present invention also relates to said QD-LEC, characterized in that the at least one small molecule organic functional material is selected from fluorescent emitters.

In principle any fluorescent emitter known to one skilled in the art can be used for the purpose of the present invention. In general, emitter compounds tend to have an extended conjugated π-electron systems. Many examples have been published, e.g. styrylamine derivatives as disclosed in JP 2913116B and WO 2001/021729 A1, and indenofluorene derivatives as disclosed in WO 2008/006449 and WO 2007/140847.

Blue fluorescent emitters are preferably polyaromatic compounds, such as, for example, 9,10-di(2-naphthylanthracene) and other anthracene derivatives, derivatives of tetracene, xanthene, perylene, such as, for example, 2,5,8,1′-tetra-t-butylperylene, phenylene, for example 4,4′-(bis(9-ethyl-3-carbazovinylene)-1,1′-biphenyl, fluorene, arylpyrenes (US 2006/0222886), arylenevinylenes (U.S. Pat. No. 5,121,029, U.S. Pat. No. 5,130,603), derivatives of rubrene, coumarine, rhodamine, quinacridone, such as, for example, N,N′-dimethylquinacridone (DMQA), dicyanomethylenepyrane, such as, for example, 4 (dicyanoethylene)-6-(4-dimethylaminostyryl-2-methyl)-4H-pyrane (DCM), thiopyrans, polymethine, pyrylium and thiapyrylium salts, periflanthene, indenoperylene, bis(azinyl)imine-boron compounds (US 2007/0092753 A1), bis(azinyl)methene compounds and carbostyryl compounds.

Further preferred blue fluorescent emitters are described in C. H. Chen et al.: “Recent developments in organic electroluminescent materials” Macromol. Symp. 125, (1997), 1-48 and “Recent progress of molecular organic electroluminescent materials and devices” Mat. Sci. and Eng. R, 39 (2002), 143-222.

Preferred fluorescent dopants according to the present invention are selected from the class of the monostyrylamines, the distyrylamines, the tristyrylamines, the tetrastyrylamines, the styrylphosphines, the styryl ethers and the arylamines.

A monostyrylamine is taken to mean a compound which contains one substituted or unsubstituted styryl group and at least one, preferably aromatic, amine. A distyrylamine is taken to mean a compound which contains two substituted or unsubstituted styryl groups and at least one, preferably aromatic, amine. A tristyrylamine is taken to mean a compound which contains three substituted or unsubstituted styryl groups and at least one, preferably aromatic, amine. A tetrastyrylamine is taken to mean a compound which contains four substituted or unsubstituted styryl groups and at least one, preferably aromatic, amine. The styryl groups are particularly preferably stilbenes, which may also be further substituted. The corresponding phosphines and ethers are defined analogously to the amines. For the purposes of this invention, an arylamine or an aromatic amine is taken to mean a compound which contains three substituted or unsubstituted aromatic or heteroaromatic ring systems bonded directly to the nitrogen. At least one of these aromatic or heteroaromatic ring systems is preferably a condensed ring system, preferably having at least 14 aromatic ring atoms. Preferred examples thereof are aromatic anthracene-amines, aromatic anthracene-diamines, aromatic pyrene-amines, aromatic pyrene-diamines, aromatic chrysene-amines and aromatic chrysene-diamines. An aromatic anthracene-amine is taken to mean a compound in which one diarylamino group is bonded directly to an anthracene group, preferably in the 9 position. An aromatic anthracene-diamine is taken to mean a compound in which two diarylamino groups are bonded directly to an anthracene group, preferably in the 9,10-position. Aromatic pyrene-amines, pyrene-diamines, chrysene-amines and chrysene-diamines are defined analogously thereto, where the diarylamino groups on the pyrene are preferably bonded in the 1 position or in the 1,6-position.

Further preferred fluorescent dopants are selected from indenofluorene-amines and indenofluorene-diamines, for example in accordance with WO 2006/122630, benzoindenofluorene-amines and benzoindenofluorene-diamines, for example in accordance with WO 2008/006449, and dibenzoindenofluorene-amines and dibenzoindenofluorene-diamines, for example in accordance with WO 2007/140847.

Examples of dopants from the class of the styrylamines are substituted or unsubstituted tristilbene-amines or the dopants described in WO 2006/000388, WO 2006/058737, WO 2006/000389, WO 2007/065549 and WO 2007/115610. Distyrylbenzene and distyrylbiphenyl derivatives are described in U.S. Pat. No. 5,121,029. Further styrylamines are found in US 2007/0122656 A1.

Particularly preferred styrylamine dopants and triarylamine dopants are the compounds of the formulae (53) to (58) and as disclosed in U.S. Pat. No. 7,250,532 B2, DE 102005058557 A1, CN 1583691 A, JP 08053397 A, U.S. Pat. No. 6,251,531 B1, and US 2006/210830 A.

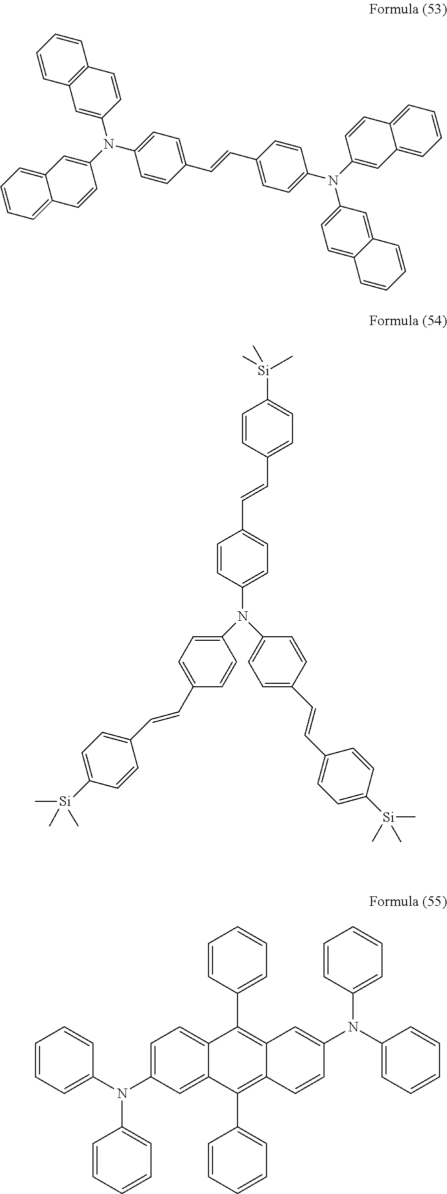

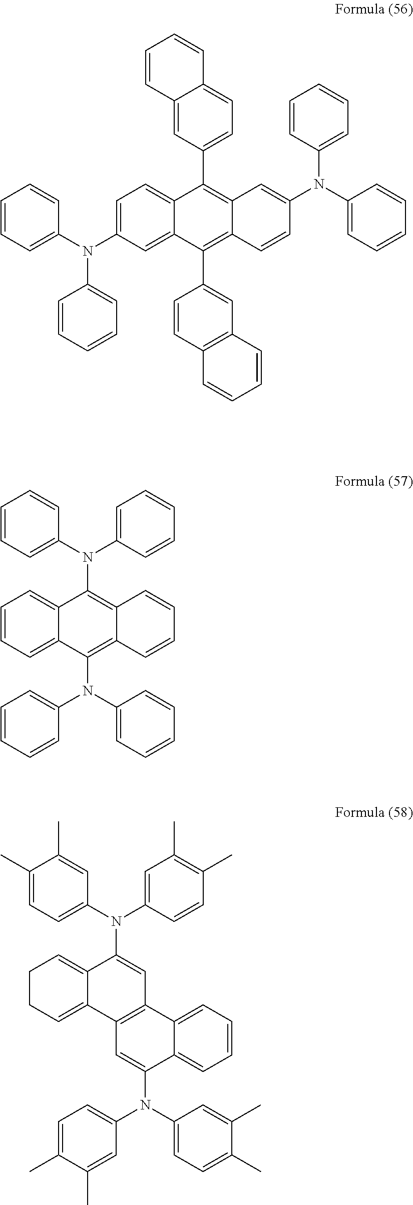

Further preferred fluorescent dopants are selected from the group of triarylamines as disclosed in EP 1957606 A1 and US 2008/0113101 A1.

Further preferred fluorescent dopants are selected from derivatives of naphthalene, anthracene, tetracene, fluorene, periflanthene, indeno-perylene, phenanthrene, perylene (US 2007/0252517 A1), pyrene, chrysene, decacyclene, coronene, tetraphenylcyclopentadiene, penta-phenylcyclopentadiene, fluorene, spirofluorene, rubrene, coumarine (U.S. Pat. No. 4,769,292, U.S. Pat. No. 6,020,078, US 2007/0252517 A1), pyran, oxazone, benzoxazole, benzothiazole, benzimidazole, pyrazine, cinnamic acid esters, diketopyrrolopyrrole, acridone and quinacridone (US 2007/0252517 A1).

Of the anthracene compounds, particular preference is given to 9,10-substituted anthracenes, such as, for example, 9,10-diphenylanthracene and 9,10-bis(phenylethynyl)anthracene. 1,4-Bis(9′-ethynylanthracenyl)-benzene is also a preferred dopant.

The QD-LECs according to the present invention comprise 4, preferably 3, particularly preferably 2, and very particularly preferably fluorescent emitter(s). Preference is given to QD-LECs comprising one EIM. QD-LECs according to the present invention preferably comprise the fluorescent emitter in a concentration of at least 0.1 wt %, particularly preferably at least 0.5 wt %, and very particularly preferably of at least 3 wt % with respect to the total amount of the emissive layer.

In a preferred embodiment the said QD-LEDs comprise at least one small molecule organic phosphorescent emitter. Thus, the present invention also relates to said QD-LEC, characterized in that the at least one small molecule organic functional material is selected from phosphorescent emitters.

In principle any phosphorescent emitter known to one skilled in the art can be used for the purpose of the present invention.

A QD-LEC according to claim 1 or 2, characterized in that the at least one small molecule organic functional material is selected from phosphorescent emitters.

Examples of phosphorescent emitters are disclosed in the applications WO 00/70655, WO 01/41512, WO 02/02714, WO 02/15645, EP 1191613, EP 1191612, EP 1191614 and WO 2005/033244. In general, all phosphorescent complexes as used in accordance with the prior art and as are known to the person skilled in the art in the area of organic electro-luminescence are suitable, and the person skilled in the art will be able to use further phosphorescent complexes without inventive step.

The phosphorescent emitter may be a metal complex, preferably with the formula M(L)_{z}, wherein M is a metal atom, L is in each occurrence independently of one another an organic ligand that is bonded to or coordinated with M via one, two or more positions, and z is an integer≧1, preferably 1, 2, 3, 4, 5 or 6, and wherein, optionally, these groups are linked to a polymer via one or more, preferably one, two or three positions, preferably via the ligands L.

M is in particular a metal atom selected from transition metals, preferably selected from transition metals of group VIII, or lanthanoides, or actinides, particularly preferably selected from Rh, Os, Ir, Pt, Pd, Au, Sm, Eu, Gd, Tb, Dy, Re, Cu, Zn, W, Mo, Pd, Ag, or Ru, and very particularly preferably selected from Os, Ir, Ru, Rh, Re, Pd, or Pt. M may also be Zn.

Preferred ligands are 2 phenylpyridine derivatives, 7,8-benzoquinoline derivatives, 2 (2-thienyl)pyridine derivatives, 2 (1-naphthyl)pyridine derivatives or 2 phenylquinoline derivatives. All these compounds may be substituted, for example by fluoro- or trifluoromethyl substituents for blue. Auxiliary ligands are preferably acetylacetonate or picric acid.

In particular, complexes of Pt or Pd with tetradentate ligands of the formula (59) as disclosed in US 2007/0087219 A1, wherein R¹ to R¹⁴ and Z¹ to Z⁵ are as defined in the reference, Pt porphyrin complexes having an enlarged ring system (US 2009/0061681 A1) and Ir complexes are suitable, for example 2,3,7,8,12,13,17,18-octaethyl-21H,23H-porphyrin-Pt(II), tetraphenyl-Pt(II)-tetrabenzopor Phyrin (US 2009/0061681 A1), cis-bis(2-phenylpyridinato-N,C2′)Pt(II), cis-bis(2-(2′-thienyl)pyridinato-N,C3′)Pt(II), cis-bis(2-(2′-thienyl)quinolinato-N,C5′)Pt(II), (2-(4,6-difluorophenyl)-pyridinato-N,C2′)Pt(II) acetylacetonate, or tris(2-phenylpyridinato-N,C2′)-Ir(III) (Ir(ppy)₃, green), bis(2-phenylpyridinato-N,C2)Ir(III) acetylacetonate (Ir(ppy)₂ acetylacetonate, green, US 2001/0053462 A1, Baldo, Thompson et al. Nature 403, (2000), 750-753), bis(1-phenylisoquinolinato-N,C2′)(2-phenylpyridinato-N,C2′)iridium(III), bis(2-phenylpyridinato-N,C2′)(1-phenylisoquinolinato-N,C2′)iridium(III), bis(2-(2′-benzothienyl)pyridinato-N,C3′)iridium(III) acetylacetonate, bis(2-(4′,6′-difluorophenyl)pyridinato-N,C2′)iridium(III) piccolinate (Firpic, blue), bis(2-(4′,6′-difluorophenyl)-pyridinato-N,C2)Ir(III) tetrakis(1-pyrazolyl)borate, tris(2-(biphenyl-3-yl)-4-tert-butylpyridine)iridium(III), (ppz)₂Ir(5phdpym) (US 2009/0061681 A1), (45ooppz)₂Ir(5phdpym) (US 2009/0061681 A1), derivatives of 2 phenyl-pyridine-Ir complexes, such as, for example, iridium(III) bis(2-phenyl-quinolyl-N,C2′)acetylacetonate (PQIr), tris(2-phenylisoquinolinato-N,C)Ir(III) (red), bis(2-(2′-benzo[4,5-a]thienyl)pyridinato-N,C3)Ir acetylacetonate ([Btp2Ir(acac)], red, Adachi et al. Appl. Phys. Lett. 78 (2001), 1622-1624).

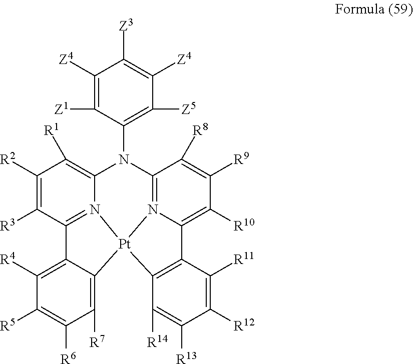

Also suitable are complexes of trivalent lanthanides, such as, for example, Tb³⁺ and Eu³⁺ (J. Kido et al. Appl. Phys. Lett. 65 (1994), 2124, Kido et al. Chem. Lett. 657, 1990, US 2007/0252517 A1), or phosphorescent complexes of Pt(II), Ir(I), Rh(I) with maleonitrile dithiolate (Johnson et al., JACS 105, 1983, 1795), Re(I) tricarbonyl diimine complexes (Wrighton, JACS 96, 1974, 998 inter alia), Os(II) complexes with cyano ligands and bipyridyl or phenanthroline ligands (Ma et al., Synth. Metals 94, 1998, 245) or Alq₃.

Further phosphorescent emitters with tridentate ligands are described in U.S. Pat. No. 6,824,895 and U.S. Pat. No. 7,029,766. Red-emitting phosphorescent complexes are mentioned in U.S. Pat. No. 6,835,469 and U.S. Pat. No. 6,830,828.

A particularly preferred phosphorescent dopant is a compound with the formula (60) and further compounds as disclosed, e.g., in US 2001/0053462 A1.

A particularly preferred phosphorescent dopant is a compound with the formula (61) and further compounds as disclosed, e.g., in WO 2007/095118 A1.

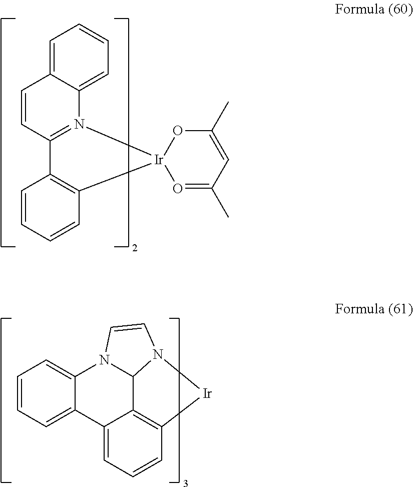

Further derivatives are described in U.S. Pat. No. 7,378,162 B2, U.S. Pat. No. 6,835,469 B2, and JP 2003/253145 A.

Particular preference is given to organic electroluminescent compounds selected from organo metallic complexes.

The term electroluminescent compound refers to a material which, upon receiving energy by applying a voltage, undergoes radiative decay to emit light.

Further to metal complexes mentioned elsewhere herein, a suitable metal complex according to the present invention can be selected from transition metals, rare earth elements, lanthanides and actinides is also subject of this invention. Preferably the metal is selected from Ir, Ru, Os, Eu, Au, Pt, Cu, Zn, Mo, W, Rh, Pd, or Ag.

In a preferred embodiment, the small organic functional material emits in ultraviolet (UV) range. Suitable UV emitter materials can be selected from organic compounds comprising a wide-gap between the highest occupied molecular orbital (HOMO) and the lowest unoccupied molecular orbital (LUMO) moieties with a small π-conjugated system. Such UV emitter can be preferably selected from small molecular compounds comprising carbazoles, indenocarbazole, indolocarbazole, silane, fluorene, triazine, thiophene, dibenzothiophene, furane, dibenzofurane, imidazole, benzimidazole, anthracene, naphthalene, phenanthrene, amine, triarylamine and derivatives thereof.

The QD-LECs according to the present invention comprise 4, preferably 3, particularly preferably 2, and very particularly preferably fluorescent emitter(s). Preference is given to QD-LECs comprising one EIM.

QD-LECs according to the present invention preferably comprise the fluorescent emitter in a concentration of at least 1 wt %, particularly preferably at least 5 wt %, and very particularly preferably of at least 10 wt % with respect to the total amount of the emissive layer.

The QD-LEC according to the present invention comprises
(1) a first electrode;
(2) a second electrode;
(3) an emissive layer (EML) comprising the at least one quantum dot, at least one ionic compound and the at least one small organic functional material positioned between the first and second electrode.

As outlined elsewhere within the present application, QD-ECs are particularly suited for the application in phototherapy and PDT. They are rather simple in terms of structure and manufacturing, which reduces production costs. More advantages of OLECs, particularly QD-LEC(s) have already been discussed within the present invention. The OLECs preferably comprise at least two electrodes, particularly preferably two electrodes, a cathode and an anode. Both electrodes are connected through the EML.

Preferred materials for the electrodes used in QD-LECs are selected from metals, particularly preferably selected from Al, Cu, Au, Ag, Mg, Fe, Co, Ni, Mn, Zn, Cr, V, Pd, Pt Ga, In and their alloys, conductive oxide, for example ITO, AZO, ZnO, and conductive organic thin films comprising such as poly(ethylenedioxythiophene)-polystyrene sulfonate (PEDOT:PSSH), Polyaniline (PANI). Further suitable conducting polymers could be found for example in the reviews edited by Michael S. Freund & Bhavana Deore, in “Self-Doped Conducting Polymers”, John Willey & Sons, Ltd., 2007.

Preferably, the QD-LECs are prepared on a flexible substrate. The suitable substrate is preferably selected from films or foils based on polymers or plastics. The main selection criteria for polymers or plastics are 1) hygienic property and 2) glass transition temperature. The glass temperature (T_{g}) of the polymers can be found in a common handbooks, e.g. in “Polymer Handbook”, Eds. J. Brandrup, E. H. Immergut, and E. A. Grulke, John Willey & Sons, Inc., 1999, VI/193-VI/276. Preferably, the T_{g} of the polymer is above 100° C., particularly preferably above 150° C., and very particularly preferably above 180° C. Very preferred substrates are for example, poly(ethylene terephthalate) (PET) and poly(ethylene 2,6-naphthalate) (PEN).

To avoid degradations caused by oxygen and moisture, and also to prevent active materials in the devices, for example the ionic compounds and the organic electroluminescent compounds from being in contact with the subject to be treated, an appropriate encapsulation for the said device is a prerequisite for the applications in therapeutic treatments and cosmetic conditions.

There are many technologies suitable for encapsulation of the devices according to the present invent. In general, all encapsulation techniques, which are developed for organic light emitting diodes (OLEDs), organic solar cells, organic dye-sensitized solar cells, organic field-effect transistor (OFETs), thin film batteries, microelectromechanical systems (MEMS) and electronic papers, can be applied in order to encapsulate the devices according to the present invention.

In a preferred embodiment, the device of the present invention is encapsulated using a thin film encapsulation. Typically, a thin film encapsulation consists of a multi alternating layers of an inorganic/organic stack, wherein inorganic layers are used to achieve adequate barrier performance and organic layers to eliminate inevitable defects of the inorganic layers. The materials used for inorganic layers can be selected from metals, metal oxides or mixed oxides, for example Ag, SiOₓ, SiNₓ, AlOₓ, ZrOₓ, ZnOₓ, HfOₓ, TiOₓ and indium tin oxide and so on. Some examples are alternating multilayers of vacuum-deposited acrylate polymers/AlOₓ as reported by Graff, G. L. et al. (J. Appl. Phys. 2004, 96, 1840), Al₂O₃/polyurea layers as reported by Young Gu Lee et al. (Org. Electron. 2009, 10, 1352 and in Dig. Tech. Pap.—Soc. Inf. Disp. Int. Symp. 2008, 39, 2011), SiON/SiO₂/parylene on PET substrate as reported by Han, Jin Woo, et al. (Jpn. J. Appl. Phys., Part 1 2006, 45, 9203), and polyacrylate(20 μm)-Ag(200 nm) as reported by Wang, Li Duo et al. (Chin. Phys. Lett. 2005, 22, 2684).

By using advanced deposition techniques, for example atomic layer deposition (ALD), plasma assisted pulsed laser deposition (PAPLD) and plasma enhanced chemical vapor deposition (PECVD), the defects in inorganic layer can be significantly reduced so that all inorganic layers can be used, for example Al₂O₃/HfO₂ nanolaminated films by ALD as reported by Chang, Chih Yu et al. (Org. Electron. 2009, 10, 1300), and SiNx/SiOx layers as reported by Li, C. Y. et al. (IEEE Electron. Compon. Technol. Conf. 2008, 58^{th}, 1819), (PECVD SiO)/poly-benzo-oxazole (PBO) by Shimooka, Y. et al. (IEEE Electron. Compon. Technol. Conf. 2008, 58^{th}, 824), nanolaminated alternating layers of Al₂O₃/ZrO₂ by Meyer, J. et al. (Appl. Phys. Lett. 2009, 94, 233305/1), and nanolaminates of Al₂O₃/ZrO₂ by PAPLD as reported by Gorrn, Patrick et al. (J. Phys. Chem. 2009, 113, 11126), and SiC layers by PECVD as reported by Weidner, W. K. et al. (Annu. Tech. Conf. Proc—Soc. Vac. Coaters 2005, 48^{th}, 158), multilayer stack of silicon nitride-silicon oxide-silicon nitride silicon oxide-silicon nitride (NONON) by PECVD as reported by Lifka, H., et al. (Dig. Tech. Pap.—Soc. Inf. Disp. Int. Symp. 2004, 35, 1384), and polyethersulfon (PES)/ALD AlOₓ as reported by Park, Sang-Hee Ko, et al. (ETRI Journal 2005, 545). A review on thin film encapsulation by CVD and ALD is provided by Stoldt, Conrad R, et al. (J. Phys. D: Appl. Phys. 2006, 39, 163).

Further single layer encapsulation was also developed. Examples of single barrier layers are a perfluorinated polymer (Cytop), which can be easily spin-coated on OLEDs, as reported by Granstrom, J. et al. (Appl. Phys. Lett. 2008, 93, 193304/1), and single layer consisting of aluminum oxynitride (AlOₓN_{y}) by using a reactive radio frequency (RF) magnetron sputtering as reported by Huang, L. T. et al. (Thin Solif Films 2009, 517, 4207), single poly-SiGe layer by PECVD as reported by Rusu, Cristina et al. (J. Microelectromech. Syst. 2003, 12, 816).

Further details on materials and methods for encapsulation are disclosed, e.g., in WO 2009/089417, WO 2009/089417, WO 2009/042154, WO 2009/042052, US 2009/081356, US 2009/079328, WO 2008/140313, WO 2008/012460, EP 1868256, KR 2006/084743, KR 2005/023685, US 2005/179379, US 2005/023974, KR 2003/089749, US 2004/170927, US 2004/024105, WO 2003/070625, and WO 2001/082390.

In another preferred embodiment, the device of the present invention is encapsulated by using a curable resin together with a cap, wherein the cap covers at least the light emitting area, and the curable resin is applied between the substrate and the cap. The cap materials can be selected from metals and plastics in form of a plate or foil, and glass cap. Preferably, the cap is flexible, which is preferably selected from metal foils, plastic foils or metallised plastic foils. The metal can be selected from Al, Cu, Fe, Ag, Au Ni, whereby Al is particularly preferred. The selection criteria for plastics are 1) hygienic aspects 2) the glass transition temperature (T_{g}), which is supposed to be high enough. T_{g} of polymers can be found in a suitable handbook, for example in “Polymer Handbook”, Eds. J. Brandrup, E. H. Immergut, and E. A. Grulke, John Willey & Sons, Inc., 1999, VI/193-VI/276. Preferably, the polymer suitable for cap material has a T_{g} above 60° C., preferably above 70° C., particularly preferably above 100° C., and very particularly preferably above 120° C. The cap used in the present invention is poly(ethylene 2,6-naphthalate) (PEN).

The suitable resin can be thermally cured or UV-curable. Preferably, the resin is UV-curable, optionally supported or facilitated by heating. A typical resin is the epoxy-based resin, which is commercially available at for example Nagase & Co., LTD. and DELO Industrie Klebstoffe. The resin can be applied on full-area of the emitting area or just on the edge, where no light emitting area is underneath.

Preferably, the QD-LECs are prepared on a flexible substrate. The suitable substrate is preferably selected from films or foils based on polymers or plastics. The selection criterion for polymers or plastics are 1) hygienic property 2) glass transition temperature. The glass temperature (Tg) of the polymers can be found in a suitable handbook, for example in “Polymer Handbook”, Eds. J. Brandrup, E. H. Immergut, and E. A. Grulke, John Willey & Sons, Inc., 1999, VI/193-VI/276. Preferably, the Tg of the polymer is above 100° C., very preferably above 150° C., and particularly above 180° C. Very preferred substrates are for example, poly(ethylene terephthalate) (PET) and poly(ethylene 2,6-naphthalate) (PEN)

QD-LECs are characterized in that charge transport occurs via transport of charged species, rather than pure transport of electrons and holes as observed in OLEDs. Thus, QD-LECs typically comprise ionic species.

Typical ionic species, also called ionic materials, which are suitable for the QD-LECs according to the present invention, have the general formula K⁺A⁻, wherein K⁺ and A⁻ represent a cation and an anion, respectively.

Preferably the ionic materials are soluble in the same solvent as the organic emissive material. This easily allows the preparation of a mixture comprising the said emitter material(s) and the ionic material(s). Typically organic emissive materials are soluble in common organic solvents, such as toluene, anisole, chloroform.

Preferably, the said ionic material is solid at room temperature and particularly preferably, the said ionic material is solid at room temperature and getting softer between 30 to 37° C.

The cation can be organic or inorganic. Suitable inorganic cations K⁺ can be selected from, for example, K⁺ (potassium) and Na⁺. Suitable organic cations K⁺ can be selected from ammonium-, phosphonium, thiouronium-, guanidinium cations as shown in formulae (62) to (66) or heterocyclic cations as shown in formulae (67) to (94).

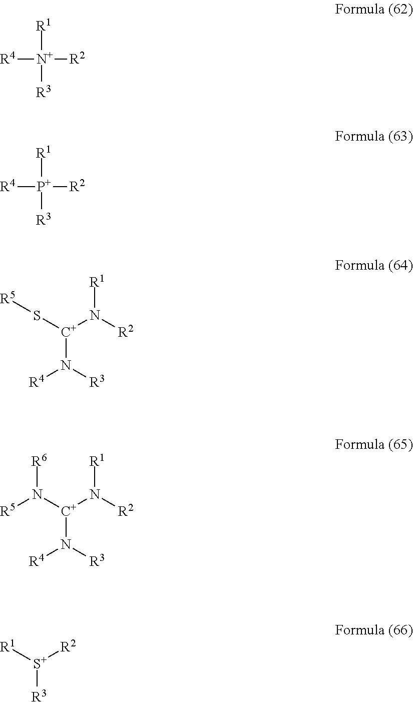

wherein

R¹ to R⁶ can be, independently from each other, selected from linear or hyperbranched alkyl rests with 1 to 20 C-atoms, linear or hyperbranched alkenyl rests with 2 to 20 C-atoms and one or more non-conjugated double bonds, linear or hyperbranched alkinyl rests with 2 to 20 C-atoms and one or more non-conjugated triple bond, saturated, partly saturated or completely saturated cycloalkyl with 3 to 7 C-atoms, which can further be substituted with alkyl groups having 1 to 6 C-atoms, wherein one or more substituents R may be partly or completely substituted with halogen, particularly with —F and/or —Cl, or partly substituted with —OR′, —CN, —C(O)OH, —C(O)NR′₂, —SO₂NR′₂, —SO₂OH, —SO₂X, —NO₂, wherein one or two non adjacent and non α-carbon atoms of R¹ to R⁶ can be substituted with groups selected from —O—, —S—, —S(O)—, —SO₂—, —N⁺R′₂⁻, —C(O)NR′—, —SO₂NR′—, and —P(O)R′—, wherein R′═H, unsubstituted, partly or completely with —F substituted C1 to C6-alkyl, C3 to C7-cycloalkyl, unsubstituted or substituted phenyl and X=halogen.

In formula (62) R¹ to R⁴ can be H, with the provisio that at least one of the rests R¹ to R⁴ is not H. In formula (63) R¹ to R⁴ can be H and NR′₂, wherein R′ is defined as above. In formula (64) R¹ to R⁵ can be H. In formula (65) R¹ to R⁶ can be H, CN, and NR′₂, wherein R′ is defined as above.

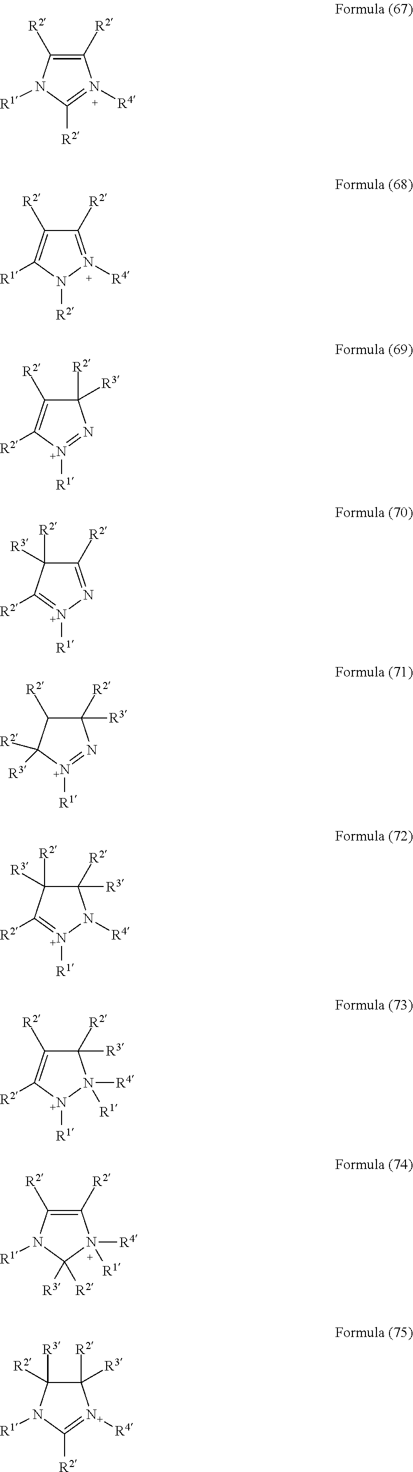

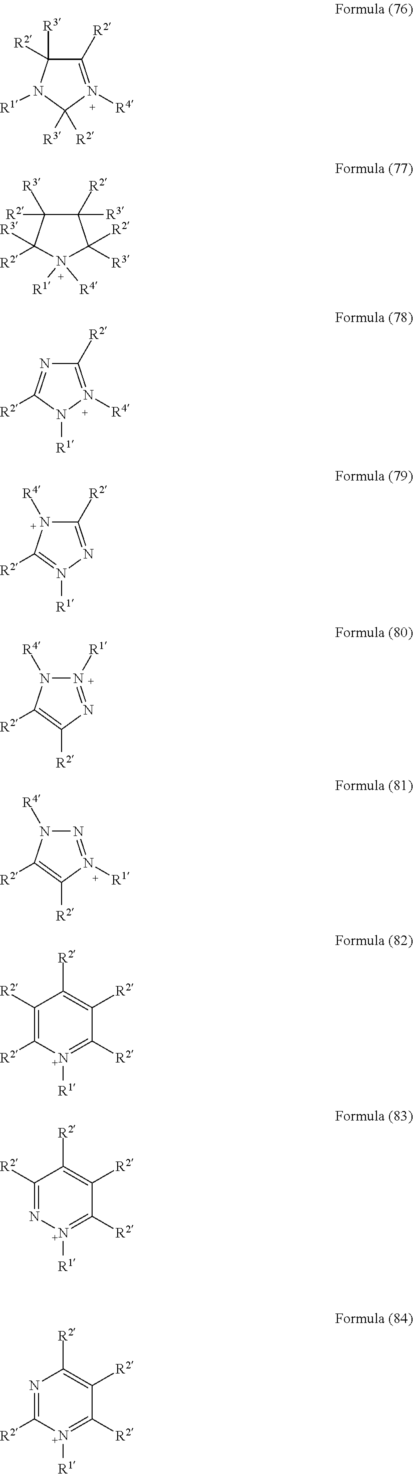

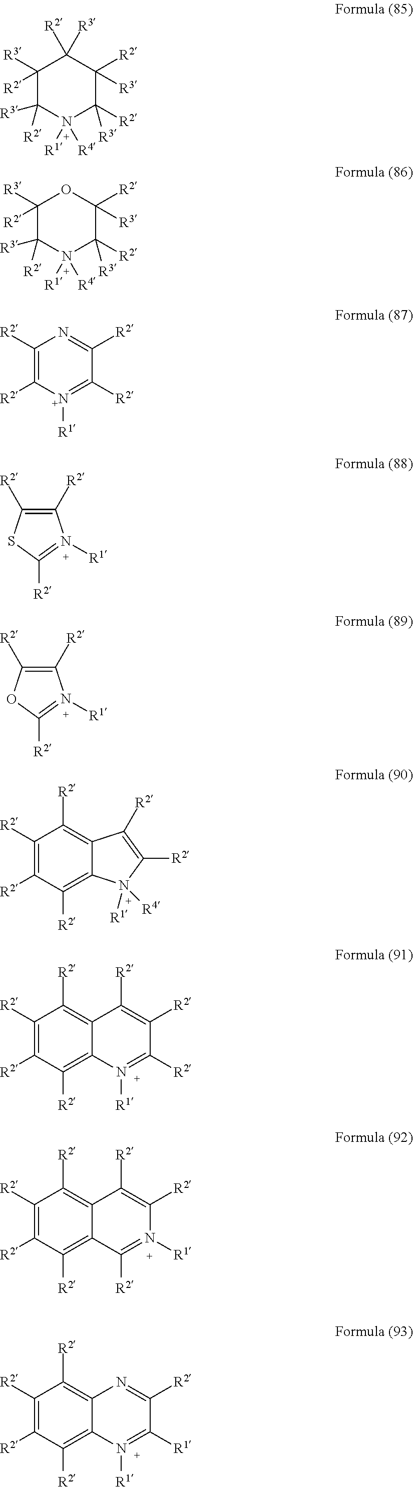

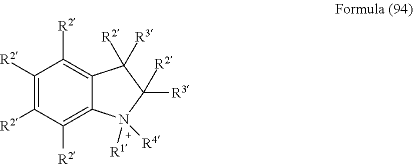

Wherein the substituents R¹′ to R⁴′ are independently from each other selected from H, CN, linear and branched alkyl rest with 1 to 20 C-atoms, linear or branched alkenyl rest with 2 to 20 C-atoms and one or more non conjugated double bonds, linear or branched alkinyl rest with 2 to 20 C-atoms and one or more non conjugated triple bonds, partly or completely non saturated cycloalkyl rest with 3 to 7 C-atoms which can be substituted with alkyl rests with 1 to 6 C-atoms, saturated and partly or completely non saturated heteroaryls, heteroaryl-C₁-C₆-alkyl, or alkyl-C₁-C₆-alkyl, wherein the substituents R¹′, R²′, R³′ and/or R⁴′ together can form a ring, wherein one or more of the substituents R¹′ to R⁴′ can partly or completely be substituted with halogen, particularly with —F and/or —Cl, and —OR′, —CN, —C(O)OH, —C(0)NR′₂, —SO₂NR′₂, —C(0)X, —SO₂OH, —SO₂X, —NO₂, wherein the substituents R¹′ and R⁴′ are not substituted with halogen at the same time, wherein one or two carbon atoms of the substituents R¹′ and R²′, which are non adjacent or bound to an heteroatom, can be substituted by a group selected from —O—, —S—, —S(O)—, —SO₂—, —N⁺R′₂—, —C(O)NR′—, —SO₂NR′—, and —P(O)R′— wherein R′═H, unsubstituted, partly or completely with —F substituted alkyl with 1 to 6 C-atoms, cycloalkyl with 3 to 7 C-atoms, unsubstituted or substituted phenyl and X=halogen.

Preference is given to R²′ selected from —OR′, —NR′₂, —C(O)OH, —C(O)NR′₂, —SO₂NR′₂)—SO₂OH, —SO₂X, and —NO₂.

Further preferred ionic materials are disclosed in, e.g., US 2007/0262694 A1.

Further particularly preferred ionic materials comprise a cation having a structure represented by formula (95). They include N,N,N-trimethylbutyl ammonium ion, N-ethyl-N,N-dimethyl-propyl ammonium ion, N-ethyl-N,N-dimethylbutyl ammonium ion, N,N,-dimethyl-N-propylbutyl ammonium ion, N-(2-methoxyethyl)-N,N-dimethylethyl ammoniumion, 1-ethyl-3-methyl imidazolium ion, 1-ethyl-2,3-dimethyl imidazoliun ion, 1-ethyl-3,4-dimethyl imidazolium ion, 1-ethyl-2,3,4-trimethyl imidazolium ion, 1-ethyl-2,3,5-trimethyl imidazolium ion, N-methyl-N-propyl pyrrolidinium ion, N-butyl-N-methylpyrrolidinium ion, N-sec-butyl-N-methylpyrrolidinium ion, N-(2-methoxyethyl)-N-methylpyrrolidinium ion, N-(2-ethoxyethyl)-N-methylpyrrolidinium ion, N-methyl-N-propyl piperidinium ion, N-butyl-N-methyl piperidinium ion, N-sec-butyl-N-methylpiperidinium ion, N-(2-methoxyethyl)-N-methyl piperidiniumion and N-(2-ethoxyethyl)-N-methyl piperidinium ion.

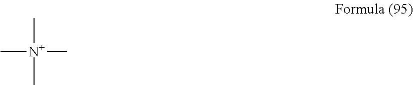

Very particularly preferred is N-methyl-N-propyl piperidinium.

Particularly preferred ionic material is a compound selected from the group of ionic compounds, which are soluble in common organic solvents such as toluene, anisole, and chloroform, consisting of methyltrioctylammonium trifluoromethane-sulfonate (MATS), 1-methyl-3-octylimidazolium octylsulfate, 1-butyl-2,3-dimethylimidazolium octylsulfate, 1-octadecyl-3-methylimidazolium bis(trifluoromethylsulfonyl)imide, 1-octadecyl-3-methylimidazolium tris(pentafluoroethyl)trifluorophosphate, 1,1-dipropylpyrrolidimium bis(trifluoromethylsulfonyl)imide, trihexyl(tetradecyl)phosphonium bis(1,2-benzenediolato(2-)-O,O′)borate, and N,N,N′,N′,N′,N′-pentamethyl-N′-propylguanidinium trifluoromethanesulfonate.

Further preferred cations are selected from compounds of one of the general formulae (96) to (101)

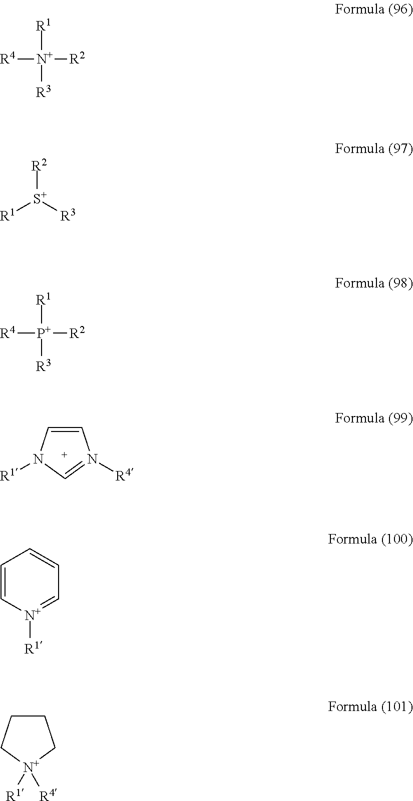

Wherein R¹ to R⁴ are defined as in formulae (62), (63), and (67), and R¹′ and R⁴′ as in formulae (68), (82), and (77).

Further preferred ionic materials suitable for the QD-LECs according to the present invention is a compound wherein one of K⁺ or A⁻ is covalently bounded to a polymer backbone.

Further preferred ionic materials suitable for the QD-LECs according to the present invention are selected from compounds wherein one of K⁺ or A⁻ is an organic emissive material, which can be selected from small molecule and polymeric emissive materials as described elsewhere within the present invention.

Suitable anions A⁻ can be selected from [HSO₄]⁻, [SO₄]²⁻, [NO₃]⁻, [BF₄]⁻, [(R_{F})BF3]⁻, [(R_{F})₂BF₂]⁻, [(R_{F})₃BF]⁻, [(R_{F})₄B]⁻, [B(CN)₄]⁻, [PO₄]³⁻, [HPO₄]²⁻, [H₂PO₄]⁻, [Alkyl-OPO₃]²⁻, [(Alkyl-O)₂PO₂]⁻, [Alkyl-PO₃]²⁻, [R_{F}PO₃]²⁻, [(Alkyl)₂PO₂]⁻, [(RF)₂PO₂]⁻, [R_{F}SO₃]⁻, [HOSO₂(CF₂)ₙSO₂O]⁻, [OSO₂(CF₂)ₙSO₂O]²⁻, [Alkyl-SO₃]⁻, [HOSO₂(CH₂)ₙSO₂O]⁻, [OSO₂(CH₂)ₙSO₂O]²⁻, [Alkyl-OSO₃]⁻, [Alkyl-C(O)O]⁻, [HO(O)C(CH₂)ₙC(O)O]⁻, [R_{F}C(O)O]⁻, [HO(O)C(CF₂)ₙC(O)O]⁻, [O(O)C(CF₂)ₙC(O)O]²⁻, [(R_{F}SO₂)₂N]⁻, [(FSO₂)₂N]⁻, [((R_{F})₂P(O))₂N]⁻, [(R_{F}SO₂)₃C]⁻, [(FSO₂)₃C]⁻, Cl⁻ and/or Br⁻

wherein:
n=1 to 8;
R_{F} is fluorinated alkyl of formula (CₘF₂ₘ₋ₓ₊₁Hₓ) with m=1 to 12 and x=0 to 7, wherein for m=1 and x=0 to 2, and/or fluorinated (also perfluorinated) aryl or alkyl-aryl.

The alkyl-group mentioned above can be selected from linear or hyperbranched alkyl groups with 1 to 20 C-atoms, preferably with 1 to 14 C-atoms and particularly preferably with 1 to 4 C-atoms. Preferably R_{F} means CF₃, C₂F₅, C₃F₇ or C₄F₉.

Preferred anions are selected from PF₆⁻, [PF₃(C₂F₅)₃]⁻, [PF₃(CF₃)₃]⁻, BF₄⁻, [BF₂(CF₃)₂]⁻, [BF₂(C₂F₅)₂]⁻, [BF₃(CF₃)]⁻, [BF₃(C₂F₅)]⁻, [B(COOCOO)₂⁻(BOB⁻), CF₃SO₃⁻(Tf⁻), C₄F₉SO₃ (Nf⁻), [(CF₃SO₂)₂N]⁻(TFSi⁻), [(C₂F₅SO₂)₂N]⁻(BETI⁻), [(CF₃SO₂)(C₄F₉SO₂)N]⁻, [(CN)₂N]⁻(DCA⁻), [CF₃SO₂]₃C]⁻, and [(CN)₃C]⁻.

Further preferred ionic materials suitable for the QD-LECs according to the present invention selected from compounds with the formula (Kⁿ⁺)ₐ(A^{m-})_{b}, wherein n, m, a, and b are integers from 1 to 3, and n×a−m×b=0 and wherein one of Kⁿ⁺ or A^{m-} is an organic emissive material, which can be selected from compound comprising groups of small molecule or polymeric emitters as outlined elsewhere within the present invention. Preferably, n. m a, b are 1.

In a preferred embodiment, in the said compound in form of (Kⁿ⁺)ₐ(A^{m-})_{b}, one of Kⁿ⁺ or A^{m-} is an emissive metal complex, and particularly preferably Kⁿ⁺ is an emissive metal complex, wherein the metal can be selected from transition metals, preferably those of group VIII elements, lanthanides, and actinides, particularly preferably selected from Rh, Os, Ir, Pt, Au, Sm, Eu, Gd, Tb, Dy, Re, Cu, W, Mo, Pd, Ag, Ru, and very particularly preferably selected from Ru, Os, Ir, Re. Some non-limiting examples for Kⁿ⁺ are [Ir(ppy)₂(bpy)]⁺, [Ir(ppy)₂(dpp)]⁺, [Ir(ppy)₂(phen)]⁺, [Ru(bpy)₃]²⁺, [Os(bpy)₂L)]²⁺ (L=cis-1,2-bis(diphenylphosphino)ethylene).

In a further embodiment of the present invention the said QD-LECs comprise a compound with the formula (Kⁿ⁺)ₐ(A^{m-})_{b}, wherein one of Kⁿ⁺ or A^{m-} is an emissive singlet emitter, and particularly preferably Kⁿ⁺ an emissive singlet emitter. Such kind of compound can be selected from charged laser dyes, for examples p-quaterphenyl-4,4′″-disulfonicacid disodiumsalt (polyphenyl 1), p-quaterphenyl-4,4′″-disulfonicacid dipotassiumsalt (polyphenyl 2), 2-(4-biphenylyl)-6-phenylbenzoxazotetrasulfonicacid potassium salt (furan 2), [1,1′-biphenyl]-4-sulfonic acid, 4′,4″-1,2-ethene-diylbis-, dipotassium salt (stilbene 1), 2,2′-([1,1′-biphenyl]-4,4′-diyldi-2,1-ethenediyl)-bis-benzenesulfonic acid disodium salt (stilbene 3), benzofuran, 2,2′-[1,1′-biphenyl]-4,4′-diyl-bis-tetrasulfonic acid (tetrasodium salt) (furan 1), 2-(p-dimethylaminostyryl)-pyridylmethyl Iodide (DASPI), 2-(p-dimethylaminostyryl)-benzothiazolylethyl Iodide (DASBTI), 3,3′-diethyloxacarbocyanine Iodide (DOCI), 4,4-difluoro-1,3,5,7,8-pentamethyl-4-bora-3a,4a-diaza-s-indacene 1,3,5,7,8-pentamethylpyrromethenedifluoroborate complex (pyrromethene 546), 3,3′-dimethyl-9-ethylthiacarbocyanine Iodide (DMETCI), disodium-1,3,5,7,8-pentamethylpyrromethene-2,6-disulfonate-difluoroborate complex (pyrromethene 556), 4,4-difluoro-2,6-diethyl-1,3,5,7,8-pentamethyl-4-bora-3a,4a-diaza-s-indacene 2,6-diethyl-1,3,5,7,8-pentamethylpyrromethenedifluoroborate complex (pyrromethene 567), o-(6-amino-3-imino-3H-xanthen-9-yl)-benzoic acid (rhodamine 110), benzoic acid, 2-[6-(ethylamino)-3-(ethylimino)-2,7-dimethyl-3H-xanthen-9-yl], perchlorate (rhodamine 19), 4,4-difluoro-2,6-di-n-butyl-1,3,5,7,8-pentamethyl-4-bora-3a,4a-diaza-s-indacene 2,6-di-n-butyl-1,3,5,7,8-pentamethylpyrromethenedifluoroborate complex (pyrromethene 580), benzoic acid, and 2-[6-(ethylamino)-3-(ethylimino)-2,7-dimethyl-3H-xanthen-9-yl]-ethyl ester, monohydrochloride (rhodamine 6G), which are commercially available at Lambda Physik AG, Goettingen, Germany.

Another subject of the present invention is said QD-LEC comprising at least one compound of the formula (Kⁿ⁺)ₐ(A^{m-})_{b}, characterized in that one of Kⁿ⁺ or A^{m-1} is an emissive singlet emitter.

Very preferably Kⁿ⁺ is an emissive singlet emitter. Kⁿ⁺ is preferably selected from the group as defined above.

Preferably the light emitting device is a electroluminescent device. Preference is given to said QD-LEC comprising 3, particularly preferably 2, and very particularly preferably 1 compound of said formula (Kⁿ⁺)ₐ(A^{m-})_{b}.

Actually, if the ionic specie is itself a light emitting material it is considered as organic functional material as defined herein. In this case no further small functional material may be needed for said QD-LECs.

In principle any quantum dot (QD) known to one skilled in the art can be employed in QD-LECs according to the present invention

Preference is given to quantum dots having emission intensity maxima in the range between 300 and 2000 nm, preferably between 350 and 1500 nm. Emission wavelengths can easily be adjusted by choosing the suitable organic semiconductor and/or by choosing the suitable quantum dot and/or by the size of a quantum dot, which in turn can precisely be tailored by synthesis. Intensities of emission can also be adapted by the concentration of a specifically sized quantum dot used in the said QD-LEC.

Preferably the QD-LEC according to the present invention comprises quantum dots selected from Group II-VI, Group III-V, Group IV-VI and Group IV semiconductors, preferably ZnO, ZnS, ZnSe, ZnTe, CdS, CdSe, CdTe, HgS, HgSe, HgTe, MgS, MgSe, GeS, GeSe, GeTe, SnS, SnSe, SnTe, PbO, PbS, PbSe, PbTe, GaN, GaP, GaAs, GaSb, InN, InP, InAs, InSb, AlN, AlP, AlAs, AlSb, GaN, GaP, GaAs, GaSb, and a combination thereof.

Suitable semiconducting materials, which can be incorporated into quantum dots, are selected from elements of Group II-VI, such as CdSe, CdS, CdTe, ZnSe, ZnO, ZnS, ZnTe, HgS, HgSe, HgTe and alloys thereof such as CdZnSe; Group III-V, such as InAs, InP, GaAs, GaP, InN, GaN, InSb, GaSb, AlP, AlAs, AlSb and alloys such as InAsP, CdSeTe, ZnCdSe, InGaAs; Group IV-VI, such as PbSe, PbTe and PbS and alloys thereof; Group III-VI, such as InSe, InTe, InS, GaSe and alloys such as InGaSe, InSeS; Group IV semiconductors, such as Si and Ge alloys thereof, and combinations thereof in composite structures.

Further suitable semiconductor materials include those disclosed in U.S. patent application Ser. No. 10/796,832 and include any type of semiconductor, including group II-VI, group III-V, group IV-VI and group IV semiconductors. Suitable semiconductor materials include, but are not limited to, Si, Ge, Sn, Se, Te, B, C (including diamond), P, BN, BP, BAs, AlN, AlP, AlAs, AlS, AlSb, BaS, BaSe, BaTe, CaS, CaSe, CaTe, GaN, GaP, GaAs, GaSb, InN, InP, InAs, InSb, AlN, AlP, AlAs, AlSb, GaN, GaP, GaAs, GaSb, ZnO, ZnS, ZnSe, ZnTe, CdS, CdSe, CdTe, HgS, HgSe, HgTe, BeS, BeSe, BeTe, MgS, MgSe, GeS, GeSe, GeTe, SnS, SnSe, SnTe, PbO, PbS, PbSe, PbTe, CuF, CuCl, CuBr, CuI, Si₃N₄, Ge₃N₄, Al₂O₃, (Al, Ga, In)₂ (S, Se, Te)₃, Al₂CO, and an appropriate combination of two or more such semiconductors.

Preferably the quantum dot is selected from Group II-VI, Group III-V, Group IV-VI and Group IV semiconductors, particularly preferably from ZnO, ZnS, ZnSe, ZnTe, CdS, CdSe, CdTe, HgS, HgSe, HgTe, MgS, MgSe, GeS, GeSe, GeTe, SnS, SnSe, SnTe, PbO, PbS, PbSe, PbTe, GaN, GaP, GaAs, GaSb, InN, InP, InAs, InSb, AlN, AlP, AlAs, AlSb, GaN, GaP, GaAs, GaSb, and a combination thereof.

In some embodiments, the quantum dots may comprise a dopant from the group consisting of: a p-type dopant or an n-type dopant. The properties and synthesis of a doped quantum dot can be referred to “n-type colloidal semiconductor nanocrystals” by Moonsub Shim & Philippe Guyot-Sionnest, Nature vol 407 (2000) p 981, and “Doped Nanocrystals” by Norris et al., Science, 319 (2008), p 1776. The quantum dots of the present invention can also comprise II-VI or III-V semiconductors. Examples of II-VI or III-V semiconductor nanocrystals include any combination of an element from Group II, such as Zn, Cd and Hg, with any element from Group VI, such as S, Se, Te, Po, of the Periodic Table; and any combination of an element from Group III, such as B, Al, Ga, In, and Tl, with any element from Group V, such as N, P, As, Sb and Bi, of the Periodic Table.

In quantum dots, photoluminescence and electroluminescence arise from the band edge states of the nanocrystal. The radiative band-edge emission from nanocrystals competes with non-radiative decay channel originating from surface electronic states, as reported by X. Peng, et al., J. Am. Chem. Soc. Vo1119:7019-7029 (1997). Thus, the presence of surface defects such as dangling bonds provides non-radiative recombination centers and lower emission efficiency. An efficient method to passivate and remove the surface trap states is to epitaxially grow an inorganic shell material on the surface of the nanocrystal, as dislcosed by X. Peng, et al., J. Am. Chem. Soc. Vo1119:7019-7029 (1997). The shell material can be chosen such that the electronic levels are type I with respect to the core material (e.g., with a larger bandgap to provide a potential step localizing the electron and hole to the core). As a result, the probability of non-radiative recombination can be reduced.

Core-shell structures are obtained by adding organometallic precursors containing the shell materials to a reaction mixture containing the core nanocrystal. In this case, rather than a nucleation-event followed by growth, the cores act as the nuclei, and the shells grow from their surface. The temperature of the reaction is kept low to favour the addition of shell material monomers to the core surface, while preventing independent nucleation of nanocrystals of the shell materials. Surfactants in the reaction mixture are present to direct the controlled growth of shell material and ensure solubility. A uniform and epitaxially grown shell is obtained when there is a low lattice mismatch between the two materials. Additionally, the spherical shape acts to minimize interfacial strain energy from the large radius of curvature, thereby preventing the formation of dislocations that could degrade the optical properties of the nanocrystal system.

In a preferred embodiment, ZnS can be used as the shell material using synthetic processes well known to one skilled in the art.

In a particularly preferred embodiment, the quantum dot of the invention comprises semiconducting materials selected from Group II-VI semiconductors, alloys thereof and core/shell structures made there from. In further embodiments, the Group II-VI semiconductors are CdSe, CdS, CdTe, ZnSe, ZnS, ZnTe, alloys thereof, combinations thereof and core/shell, core multi-shell layered-structures thereof.

In some embodiments, the quantum dots according to the present invention comprise further ligands conjugated, cooperated, associated or attached to their surface. Suitable ligands include any group known to those skilled in the art, including those disclosed in U.S. Ser. No. 10/656,910 and U.S. 60/578,236. Use of such ligands can enhance the ability of the quantum dots to incorporate into various solvents and matrix materials, including polymers. Further preferred ligands are such having a “head-body-tail” structure, as disclosed in US 2007/0034833A1, wherein further preferably the “body” has an electron or hole transport function, as disclosed in US 20050109989A1.

The term quantum dot refers to nanocrystals that are substantially mono-dispersive in size. A quantum dot has at least one region or characteristic dimension with a dimension of less than about 500 nm, and down to on the order of less than about 1 nm. The term mono-dispersive means the size distribution is within +−10% of the stated value, for example a mono-dispersive nanocrystals of 100 nm in diameter encompasses a range of sizes from 90 nm or larger to 110 nm or smaller.

Due to the finite size of the QDs, in particular core-shell QDs, they display unique optical properties compared to their bulk counterparts. The emission spectrum is defined by a single Gaussian peak, which arises from the band-edge luminescence. The emission peak location is determined by the core particle size as a direct result of quantum confinement effects. The electronic & optical properties are discussed by AI. L. Efros and M. Rosen in Annu. Rev. Mater. Sci. 2000. 30:475-521. Furthermore, the intensity of emission can be tailored according to the concentration used in the said QD-LECs, as outlined above.

The QD-LECs according to the present invention comprise as outlined elsewhere within the present invention at least one ionic species.

Preferably the at least one ionic species is selected from an ionic transition metal complex (iTMC).

One typical iTMC material is reported for example by Rudmann et al., J. Am. Chem. Soc. 2002, 124, 4918-4921 and Rothe et al., Adv. Func. Mater. 2009, 19, 2038-2044. The concentrations of the iTMC in the emissive layer (EML) can be from 1 to 50 wt %, preferably from 5 to 30 wt %, particularly preferably from 10 to 30 wt %, and very particularly preferably from 10 to 20 wt % with respect of the emissive layer.

The said QD-LECs preferably comprises further ion conducting material and/or a neutral matrix material, which can have a concentration of 1 to 90 wt %, preferably 10 to 80 wt %, particularly preferably 20 to 70 wt %, and very particularly preferably 30 to 70 wt % with respect to the total amount of the layer.

A QD-LEC according to one or more of claims 1 to 10, characterized in that at least one of the quantum dots is an ionic species.

In one preferred embodiment, the QD-LEC according to the present invention comprises a QD, which is itself an ionic compound.

Suitable ionic QD is selected from QDs comprising at least one ionic ligand (or cap). The suitable ligand for this embodiment can be preferably selected according to the general formulae (102) and (103):

[K⁺][A⁻-B-D] Formula (102)

[A⁺][K⁻-B-D] Formula (103)

wherein D is an anchor group, which anchors on the QD surface, for example a thiol group; and B a simple bond or a spacer, preferably selected from alkyl, alkoxy group; and K^{+/-} and A^{-/+} represent cations and an anions as described above.

The quantum dot comprising at least one ionic ligand according to formula (102) or (103) can be synthesized by ligand exchange as reported for example by Denis Dorokhin, et al (Nanotechnology 2010, 21, 285703). The ligand can, e.g., has the following formula (104).

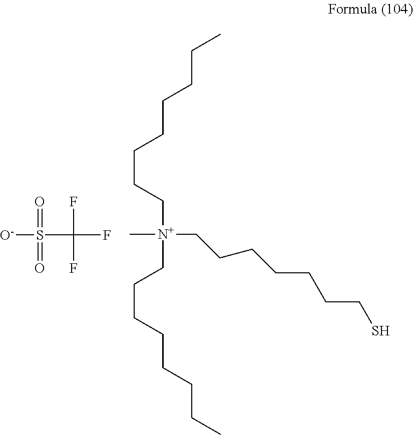 [SMILES: O=S(=O)([O-])C(F)(F)F ; CCCCCCCC[N+](C)(CCCCCCCC)CCCCCCCS]

Ligand exchange can be realized by mixing the toluene solution of trioctylphosphine oxide (TOPO)-coated core-shell CdSe/ZnS QDs with a toluene solution of ligand with formula (104) under nitrogen flow and with the help of heating for example at 40° C. By controlling the reaction time, different degree of ligand exchange, between TOPO and anion in formula (104), can be obtained. In a preferred embodiment, only partially exchange is desired, therefore, the reaction time is preferably short, for example shorter than 24 hrs.

Preference is given to QD-LECs, characterized in that the emissive layer (EML) comprises at least one ionic quantum dot and at least one small organic functional molecule selected from host materials, fluorescent emitters, phosphorescent emitters, hole transport materials (HTMs), hole injection materials (HIMs), electron transport materials (ETMs), and electron injection materials (EIMs). The small organic functional materials, which are electrically neutral, are the same as outlined elsewhere within the present invention.

Particular preference is given to said QD-LECs where the EML comprises 2, and very particularly preferably 1 ionic quantum dot(s).

In yet another preferred embodiment the EML of the QD-LECs comprises one ionic quantum dot and one small organic functional material selected from host and/or phosphorescent emitters. The concentrations of the components in the EML can be for quantum dot from 1 to 20 wt %, for host from 50 to 98 wt %, and for phosphorescent emitter from 1 to 20 wt %.

In one further preferred embodiment the EML of the QD-LECs comprises one ionic quantum dot and one small organic functional material selected from host and/or fluorescent emitters. The concentrations of the components in the EML can be for quantum dot from 1 to 20 wt %, for host from 50 to 98 wt %, and for fluorescent emitter from 1 to 20 wt %.

The EML of the QD-LECs may comprises further organic functional materials, which can be small molecule or polymer.

The present invention also relates to an ionic quantum dots, characterized in that it comprises at least one ionic ligand according to the formulae (102) or (103).

The present invention further related to a mixture used in and according to embodiments described herein, which comprises at least one quantum dot and at least one ionic compound and at least small organic functional material.

In a preferred embodiment, the said mixture comprises at least one QD, at least one ionic compound, at least one host material and at least one emitter, which can be selected from phosphorescent emitter or fluorescent emitter.

In another preferred embodiment, the said mixture comprises at least one ionic QD, at least one host material and at least one emitter, which can be selected from phosphorescent emitter or fluorescent emitter.

In yet another preferred embodiment, the said mixture comprises at least one QD, at least one host material and at least one ionic emitter, which can be selected from phosphorescent emitter or fluorescent emitter. Preferably, the said ionic emitter is selected from iTMCs.

In a further preferred embodiment, the mixture comprises at least ion conducting material, which can be selected from for example polyethylene oxides (PEO) for Li⁺.

In the said mixture, it may further comprise other organic functional material, which can in form of small molecule or polymer or oligomer or dendrimer, and can be selected from host, emitter, HIM, HTM, ETM, EIM, and metal complexes.

In the mixture according to any embodiment described herein, the QD may include at least one element selected from selected from Group II-VI, Group III-V, Group IV-VI and Group IV semiconductors, preferably ZnO, ZnS, ZnSe, ZnTe, CdS, CdSe, CdTe, HgS, HgSe, HgTe, MgS, MgSe, GeS, GeSe, GeTe, SnS, SnSe, SnTe, PbO, PbS, PbSe, PbTe, GaN, GaP, GaAs, GaSb, InN, InP, InAs, InSb, AlN, AlP, AlAs, AlSb, GaN, GaP, GaAs, GaSb, and an appropriate combination of two or more such semiconductors, and/or with core/shell, core multi-shell layered-structures thereof.

The mixture according to any embodiment described herein may be characterized in that the concentration of the QD is chosen preferably from 0.5 to 30 wt %, particularly preferably from 1 to 20 wt %, and very particularly preferably from 5 to 15 wt %.

The mixture according to any embodiment described herein may include at least further one emitter. In the mixture according to any embodiment described herein the emission spectrum of the quantum dot may overlap with the absorption of the further emitter. Thereby, a Förster energy transfer can be realized. In the mixture according to any embodiment described herein, the further emitter can be selected from organic compounds or other quantum dots.

According to a further embodiment, an electronic device includes a mixture according to any embodiment described herein or an ionic quantum dots according to any embodiment described herein. The electronic device may include at least one anode, one cathode and a functional layer in-between the anode and the cathode, wherein the functional layer includes the mixture or the quantum dot. The electronic device may be characterized in that the device is a light emitting, light converting, light harvesting, or light sensor device selected from organic light emitting diodes (OLED), polymer light emitting diodes (PLED), organic light emitting electrochemical cells, organic field effect transistors (OFET), thin film transistors (TFT), organic solar cells (O-SC), organic laser diodes (O-laser), organic integrated circuits (O-IC), radio frequency identification (RFID) tags, photodetector, sensors, logic circuits, memory elements, capacitor, charge injection layers, Schottky diodes, planarising layers, antistatic films, conducting substrates or patterns, photoconductors, electrophotographic elements, organic light emitting transistors (OLET), organic spintronic devices, and an organic plasmon emitting devices (OPEDs), preferably selected from organic light emitting diodes.

Another aspect of the invention relates to a formulation, preferably a solution, comprising a mixture or an ionic quantum dots according to any embodiment described herein and one or more organic solvents;

Examples of suitable and preferred organic solvents include, without limitation, dichloromethane, trichloromethane, monochlorobenzene, o-dichlorobenzene, tetrahydrofuran, anisole, morpholine, toluene, o-xylene, m-xylene, p-xylene, 1,4-dioxane, acetone, methylethylketone, 1,2-dichloroethane, 1,1,1-trichloroethane, 1,1,2,2-tetrachloroethane, ethyl acetate, n-butyl acetate, dimethylformamide, dimethylacetamide, dimethylsulfoxide, tetralin, decalin, indane and/or mixtures thereof. The concentration of the mixture in the solution is preferably 0.1 to 10 wt %, particularly preferably 0.5 to 5 wt %. Optionally, the solution also comprises one or more binders to adjust the rheological properties, as described in WO 2005/055248 A1.

After the appropriate mixing and ageing, solutions are evaluated as one of the following categories: complete solution, borderline solution or insoluble. The contour line is drawn to outline the solubility parameter-hydrogen bonding limits dividing solubility and insolubility. ‘Complete’ solvents falling within the solubility area can be chosen from literature values such as published in “Crowley, J. D., Teague, G. S. Jr and Lowe, J. W. Jr., Journal of Paint Technology, 38, No 496, 296 (1966)”. Solvent blends may also be used and can be identified as described in “Solvents, W. H. Ellis, Federation of Societies for Coatings Technology, 9-10, 1986”. Such a procedure may lead to a blend of ‘non’ solvents that will dissolve the mixture, although it is desirable to have at least one true solvent in a blend.

Another preferred form of a formulation according to the present invention is an emulsion, and very preferably a mini-emulsion, which are specially formulated heterophase systems in which stable nanodroplets of one phase are dispersed in a second, continuous phase. The present invention relates to a mini-emulsion, wherein the different components of the mixture are located either in the same phase or in the different phases. Preferred distributions are as follows:
1) majority or all of QDs and organic functional materials in nanodroplets (discontinuous phase), and majority or all of ionic compounds in the continuous phase;
2) majority or all of organic functional materials in nanodroplets (discontinuous phase), and majority or all of QD and ionic compounds in the continuous phase;

Both mini-emulsion, wherein the continuous phase is a polar phase, and inverse miniemulsion, wherein the continuous phase is a non-polar phase, could be used in the present invention. The preferred form is the mini-emulsion. To increase the kinetic stability of the emulsion, surfactant(s) could be added. The selection of solvents for two phase and surfactants, and the processing to make a stable mini-emulsion is well known to one skilled in the art, or are referred to various publications, for example, Landfester et al. (Annu. Rev. Mater. Res. 2006, 36, 231).

For use as thin layers in electronic or opto-electronic devices the mixture or a formulation of them of the present invention may be deposited by any suitable method. Liquid coating of devices such as light emitting device is more desirable than vacuum deposition techniques. Solution deposition methods are particularly preferred. Preferred deposition techniques include, without limitation, dip coating, spin coating, ink jet printing, letter-press printing, screen printing, doctor blade coating, roller printing, reverse-roller printing, offset lithography printing, flexographic printing, web printing, spray coating, brush coating or pad printing, slot-die coating. Ink-jet printing is particularly preferred as it allows high resolution displays to be prepared.

Selected solutions of the present invention may be applied to prefabricated device substrates by ink jet printing or microdispensing. Preferably industrial piezoelectric print heads such as but not limited to those supplied by Aprion, Hitachi-Koki, InkJet Technology, On Target Technology, Picojet, Spectra, Trident, Xaar may be used to apply the organic semiconductor layer to a substrate. Additionally semi-industrial heads such as those manufactured by Brother, Epson, Konica, Seiko Instruments Toshiba TEC or single nozzle microdispensers such as those produced by Microdrop and Microfab may be used.

In order to be applied by ink jet printing or microdispensing, the mixture of the present invention should be first dissolved in a suitable solvent. Solvents must fulfil the requirements stated above and must not have any detrimental effect on the chosen print head. Additionally, solvents should have boiling points>100° C., preferably >140° C. and more preferably >150° C. in order to prevent operability problems caused by the solution drying out inside the print head. Apart from the solvents mentioned above, suitable solvents include substituted and non-substituted xylene derivatives, di-C₁₋₂-alkyl formamide, substituted and non-substituted anisoles and other phenol-ether derivatives, substituted heterocycles such as substituted pyridines, pyrazines, pyrimidines, pyrrolidinones, substituted and non-substituted N,N-di-C₁₋₂-alkylanilines and other fluorinated or chlorinated aromatics.

A preferred solvent for depositing mixture of the present invention by ink jet printing comprises a benzene derivative which has a benzene ring substituted by one or more substituents wherein the total number of carbon atoms among the one or more substituents is at least three. For example, the benzene derivative may be substituted with a propyl group or three methyl groups, in either case there being at least three carbon atoms in total. Such a solvent enables an ink jet fluid to be formed comprising the solvent with the polymer, which reduces or prevents clogging of the jets and separation of the components during spraying. The solvent(s) may include those selected from the following list of examples: dodecylbenzene, 1-methyl-4-tert-butylbenzene, terpineol limonene, isodurene, terpinolene, cymene, diethylbenzene. The solvent may be a solvent mixture, that is a combination of two or more solvents, each solvent preferably having a boiling point>100° C., more preferably >140° C. Such solvent(s) also enhance film formation in the layer deposited and reduce defects in the layer.

The ink jet fluid (that is mixture of solvent, binder and the mixture) preferably has a viscosity at 20° C. of 1 to 100 mPa·s, particularly preferably 1 to 50 mPa·s and very particularly preferably 1 to 30 mPa·s.

The mixture or a formulation of them according to the present invention can additionally comprise one or more further components like for example surface-active compounds, lubricating agents, wetting agents, dispersing agents, hydrophobing agents, adhesive agents, flow improvers, de-foaming agents, deaerators, diluents which may be reactive or non-reactive, auxiliaries, colorants, dyes or pigments, sensitizers, stabilizers, or inhibitors.

In another embodiment, the formulation of any other embodiment described herein can be used in the manufacture of an opto-electronic device, preferably an electroluminescent device.

The QD-LECs according to the present invention may be incorporated into a device. The device may be required in order to fulfill further functions or allow interaction of different components of the device. The device comprising said QD-LEC(s) may be employed in different fields of application such as display, general lighting, backlit for displays, and in phototherapy and/or PDT. Thus, the present invention also relates to a device comprising at least one QD-LEC according to the present invention.

The device can have any shape, be rigid or flexible. The device requires energy supply in any form. The energy supply may be directly associated to the device or separated by, e.g., a cable. A battery, particularly a printable battery, may be attached to the device in order to provide a device which is comfortable for the subject to be treated forming a totally self-contained portable unit. Irradiation may, thus, occur at any time and at any place without disturbing the subject to be treated in its habits or daily life. Home use of devices according to the present invention is particularly preferable. The device may be self adhesive and detachable. It may conform a planar or non-planar portion of the body or be an implantable probe.

The device may comprise an interactive steering unit. The steering unit may allow a switch from continuous illumination to pulsed illumination. It also may allow the precise adaptation of irradiation intensities and/or wavelengths to be emitted. The steering unit may be directly associated to the device. It can also be separated via a permanent or temporary linkage. The device may be disposable and is suitable for uses in the hospital or outside the hospital.

In any case the device according to the present invention is suitable as light weight device for portable use. However, stationary devices can also be prepared. The device is sufficiently portable to enable ambulatory treatment i.e. treatment in which the subject can move around freely. It can be subsequently removed in the human subject's own time, so that treatment could take place almost everywhere and anytime. This results in a better convenience and lower costs (from avoiding either an out-patient or inpatient stay in hospital).

In the case of PDT the treatment is often associated with pain. Ambulatory devices according to the present invention can be used with lower light levels since exposure can occur for a longer period of time. This overcomes a problem of pain induced in some patients by the high irradiances from conventional sources used in hospitals. In addition lower irradiance is more effective in PDT due to reduction of the extent of photobleaching of the photopharmaceutical.

The devices may be provided with a photochemical and/or a photopharmaceutical preparation present. This may be in the form of a gel, ointment or cream. Alternatively, or as well, the device may be provided with a thin film impregnated with the photopharmaceutical. Typically, the photopharmaceutical preparation is provided as a layer in contact with the light source. Provided that the photopharmaceutical preparation is transparent or sufficiently translucent for the frequency of stimulating light, the resulting device can be readily applied without a separate step of applying the photopharmaceutical to a patient. Creams which would scatter the light may nevertheless be used if they are absorbed before the light source is switched on. A photopharmaceutical layer may be covered by a peelable release medium, such as a silicone-backed sheet. The photopharmaceutical preparation may comprise an inactive compound which is metabolised in vivo to an active compound. Delivery of the photopharmaceutical can be assisted by iontophoresis.

The output of light from the organic light-emitting semiconductor may be pulsed and an electronic control circuit or microprocessor may be provided to control this pulsing and/or other aspects of device function such as duration of exposure(s) of the area to be treated and the intensity of emitted light. Pulsed devices may be provided with a preparation of a photochemical and/or a photopharmaceutical substance which is photobleachable or which is metabolised in vivo to a photobleachable chemical species.

The output of the device may take the form of a train of pulses, preferably in which the duration of the pulses is substantially the same as the interval between successive pulses. The period of the pulse train may, for example, be in the range of 20 ms to 2000 s, depending on the photobleaching characteristics of said substance.

Preferably, the attachment means comprises an adhesive surface to enable the device to be attached to a patient.

Preferably, the ambulatory device is provided with a photochemical and/or a photopharmaceutical preparation present. Preferred features of the preparation and its delivery are as above. In particular, the photochemical and/or photopharmaceutical may be photobleachable or may be metabolized in vivo to a photobleachable chemical species.

The means for activating and deactivating the source may control other aspects of device function such as duration of exposure(s) of the area to be treated and the intensity of emitted light.

The control means may to advantage be operable to cover the source to emit a pulse train having any one or more of the preferred features of the pulse train produced by a device in accordance with the first aspect of the invention.

Suitable devices according to the present invention are selected from sleeves, bandages, pads, plaster, implantable probes, nasogastric tubes, chest drains, stents, clothe like devices, blankets, sleeping bags, devices fitting one or more teeth in the mouth, and patches.

The device may be used as a stent, for example a tube of 1.25 to 2.25 cm radius of say 10 to 12 cm length for use inside the esophagus.

The device may be a blanket or sleeping bag in order to treat, e.g., jaundice of infants. Currently infants suffering from jaundice are separated from their parents and illuminated in incubators blindfold. This represents an unpleasant situation for both the infant and the parents. In addition, the infant is not able to adjust his body temperature as easily as adults can do and overheating in the incubator is a critical issue. Flexible blankets and sleeping bag provide a way to treat the infant without these problems. The infant covered by the blanket or sleeping bag can be irradiated while laying in his parents' arms and overheating of the infant's body is not as critical as compared to traditional therapies. This is due to the fact that the devices according to the present invention require less power and produce, in turn, less heat.

In psoriatic patients plaques are often found in body folds. Conventional phototherapy represents a problem which is due to the fact that light emitted by a light source does not reach the plaque in the body folds. OLEDs theoretically offer the opportunity to design a light source with direct contact to the psoriatic skin in the body fold. As outlined above curved surfaces represent a technical difficulty when manufacturing OLEDs. The problem can, however, be solved with QD-LECs. QD-LECs can be designed to fit into body folds in order to treat psoriasis and other diseases and/or conditions found in body folds.

Devices can generally spoken individually tailored in any form that is required for treatment.

The device itself may comprise a therapeutic agent which is released in a controlled way during the treatment.

Preferably the said device comprise a plastic ionic material as described above, which has a glass transition temperature T_{g} or melting point in the range between 25 and 45° C. Thus, the device will getting softer when attached to the skin in order to get a better contact to the skin.

In a further preferred embodiment the device according to the present invention is an ambulatory device.

The present invention also relates to a device, characterized in that it comprises an attachment means for attaching the device to a patient.

The device can be self adhesive or can be temporarily fixed at the side of action with an auxiliary material such as a glue strip. The said device is characterized in that it can be a plaster, bandage, blanket, sleeping bag, sleeve, implantable probe, nasogastric tube, chest drain, pad, stent, and patch. The form and shape of the device can be tailored according to the individual needs of the treatment and according to the constitution of the subject to be treated.

The present invention also relates to a device according to this invention, characterized in that the device comprises a power supply unit or an interface for a external power supply. As outlined above the power supply can be a directly attached to the device. This allows the design of ultra-thin devices which, e.g., can be used under the clothes without disturbing the subject to be treated. The power supply can also be in a more separated unit which is connected to the device in any possible way in order to supply the power.

The device according to the present invention is intended to illuminate parts of the subject. A device characterized in that the device is used in the treatment and/or prophylaxis therapeutic and/or cosmetic diseases and conditions in animals and humans.

The device according to the present invention emits electromagnetic radiation to cause said treatment and/or prophylaxis of the area, wherein the QD-LEC(s) has an extent of at least 0.5 cm². The QD-LECs can be continuous or discontinuous. The QD-LEC(s) and its illuminating area can adopt any shape that is suitable for the treatment. This can, in particular in therapeutic conditions, prevent side effects through the irradiation of parts of the subject whose treatment is not required.

In a further preferred embodiment the device of the present invention has an extent between 0.5 cm² and 100000 cm², particularly preferably between 0.5 cm² and 50000 cm².

The QD-LECs and/or devices according to the present invention can be used to treat medical and/or cosmetic conditions. Thus, another subject of the present invention is the use of said QD-LECs and/or devices comprising them for the treatment and/or prophylaxis and/or diagnosis of diseases and/or cosmetic conditions.

Hereby any therapeutic strategy is included, ie. treatment of a subject with light can be performed with or without a combination with other treatment approaches. Treatment can, for example, be carried out with one or more wavelengths in one or more devices comprising the QD-LEC(s) of the present invention. Furthermore, in addition to devices comprising said QD-LECs, further light sources using different technologies can be used for the treatment, such as LEDs, OLEDs, and lasers. In addition, the treatment with said QD-LEC(s) and/or devices comprising them can be combined with any known treatment strategy using drugs and cosmetics.

If phototherapy is combined with the treatment of chemical compounds such as a drugs and/or cosmetics light can be used to initiate a (photo-) chemical reaction or activation of the chemical compounds, which is called photodynamic therapy (PDT). Phototherapy according to the present invention can also be used in conjunction with chemical compounds without initiating a photochemical reaction or activation. Synergistic effects for the effectiveness and safety of the treatment of a therapeutic disease can arise from sequential, parallel, and overlapping treatment with both light therapy and drugs and/or cosmetics. The drug(s) or cosmetic compound(s), e.g., can be administered first for a specific time period followed by the application of phototherapy using the QD-LECs according to the present invention or devices comprising them. The time gap between both treatments may also vary, depending on the drug, its photoreactivity, individual circumstances of the subject, and the specific disease or condition. Both treatments may also overlap timely either partly or completely. The exact treatment strategy will depend on the individual circumstances and the severity of the disease or condition.

The combination therapy can have a synergistic effect and can reduce the side effects of traditional treatment strategies (e.g. the side effects of tetracyclines). This is, at least in part, due to the fact, that smaller doses of the drugs may be required when following the combined approach as outlined herein.

Many diagnostic devices comprise light sources for either illumination only or as functional component for the diagnosis itself, e.g. for the determination of blood parameters such as oxygen. Thus the present invention also relates to said QD-LECs for diagnostic purposes. The use of light sources comprising the said QD-LECs for diagnostic purposes is also subject of the present invention. Based on the teaching of the present invention, one skilled in the art will have no problems to develop diagnostic devices for which light sources are required comprising the said QD-LECs.

Treatment is any exposure of a subject to the radiation of said QD-LECs. The treatment may be performed by direct contact between the subject and the device comprising the QD-LEC(s) or without direct contact between them. The treatment may be outside or inside the subject. Treatment outside the subject may be, for instance, treatment of the skin, wounds, eye, gingival, mucosa, tongue, hair, nail bed, and nails. Treatment inside the subject may be, for instance, blood vessels, heart, breast, lung, or any other organ of the subject. Particular devices are required for most applications inside the subject. One such example may be a stent comprising one or more QD-LECs according to the present invention. The said subject may preferably be a human or an animal. The term cosmetic also includes aesthetic applications.

The wavelength of light that is emitted by the QD-LEC(s) and/or devices when incorporated in any kind of electronic device can be precisely tailored by the selection of the appropriate components of the QD-LECs. This includes, as outlined above, the specific design of the quantum dots and the use of different emitters or colour filter and colour converter. Depending on the application of the QD-LEC(s) each therapeutic or cosmetic treatment requires a more or less defined wavelength or spectrum of wavelengths to be emitted.

The QD-LEC(s) preferably emits light and or irradiation in the range between 200 and 1000 nm, preferably between 300 and 1000 nm, particularly preferably between 300 and 950 nm, and very particularly preferably between 400 and 900 nm.

As outlined above one effect of phototherapy is the stimulation of metabolism in the mitochondria. After phototherapy, the cells show an increased metabolism, they communicate better and they survive stressful conditions in a better way.

The said QD-LEC(s) and/or the said devices comprising them can be used for cellular stimulation. Preferred wavelengths or ranges of wavelengths for cellular stimulation are in the range between 600 to 900 nm, particularly preferable between 620 and 880 nm, and very particularly preferably between 650 and 870 nm. Examples of particularly preferred wavelengths for cellular stimulation are 683.7, 667.5, 772.3, 750.7, 846, and 812.5 nm.

Any therapeutic disease and/or cosmetic condition approachable by phototherapy can be treated with QD-LEC(s) according to the present invention and said devices. These diseases and/or conditions include, e.g., skin diseases, and skin-related conditions including skin-ageing, and cellulite, enlarged pores, oily skin, folliculitis, precancerous solar keratosis, skin lesion, aging, wrinkled and sun-damaged skin, crow's feet, skin ulcers (diabetic, pressure, venous stasis), acne rosacea lesions, cellulite; photomodulation of sebaceous oil glands and the surrounding tissues; reducing wrinkles, acne scars and acne bacteria, inflammation, pain, wounds, psychological and neurological related diseases and conditions, edema, Pagets disease, primary and metastatic tumors, connective tissue disease, manipulation of collagen, fibroblast, and fibroblast derived cell levels in mammalian tissue, illuminating retina, neoplastic, neovascular and hypertrophic diseases, inflammation and allergic reactions, perspiration, sweating and hyperhydrosis from eccrine (sweat) or apocrine glands, jaundice, vitiligo, ocular neovascular diseases, bulimia nervosa, herpes, seasonal affective disorders, mood, sleep disorders, skin cancer, crigler naijar, atopic dermatitis, diabetic skin ulcers, pressure ulcers, bladder infections, relief of muscular pains, pain, stiffness of joints, reduction of bacteria, gingivitis, whitening teeth, treatment of teeth and tissue in mouth, wound healing.

Cosmetic conditions are preferably selected from acne, skin rejuvenation and skin wrinkles, cellulite, and vitiligo. Many therapeutic treatments also have cosmetic component. Psoriasis, e.g., can be mild, mild-to-moderate, moderate, moderate-to-severe and severe. Any of these categories has a cosmetic component, which may be responsible for severe psychological problems of affected patients.

Preferably the said QD-LEC(s) is used for the treatment and/or prophylaxis of humans and/or animals. Preferably the QD-LEC(s) according to the present invention is used for the treatment and/or prophylaxis of humans.

Further subjects suitable to be treated by the irradiation with QD-LEC(s) and/or devices according to the present invention are plants, microbes, bacteria, fungi, and liquids. Microbes include, but are not limited to, prokaryotes such as bacteria and archaea and eukaryotes such as protists, animals, fungi and plants. Preferred liquids are beverages and particularly preferably water.

Preference is given to the use of QD-LEC and/or devices comprising them for the treatment and/or prophylaxis and/or diagnosis of skin diseases and/or cosmetic skin conditions.

Skin as used herein is defined as the largest organ of the integumentary system including hair, scales, feathers and nails. The term skin also includes the tongue, mucosa and gingival.

As already mentioned, principally any therapeutic and cosmetic condition that is approachable by phototherapy is covered by the present invention. The distinction between the terms therapeutic and cosmetic depends, as outlined above, on individual circumstances, the severity of the condition and the assessment of the physician. As outlined in this invention many therapeutic conditions are associated with cosmetic effects, independent of the severity of the therapeutic disease.

The skin diseases and skin related conditions include, but are not limited to acneform eruptions, autoinflammatory skin diseases or conditions, chronic blistering, conditions of the mucous membranes, conditions of the skin appendages, conditions of the subcutaneous fat, connective tissue diseases, abnormalities of dermal fibrous and elastic tissue, dermal and subcutaneous growths, dermatitis, atopic dermatitis, contact dermatitis, eczema, pustular dermatitis, seborrheic dermatitis and eczema, disturbances of pigmentation, drug eruptions, endocrine-related diseases and conditions, epidermal nevi diseases and conditions, neoplasms, cysts, erythemas, genodermatoses, infection-related diseases and conditions, bacterium-related diseases and conditions, mycobacterium-related diseases and conditions, mycosis-related diseases and conditions, parasitic infestations, stings, and bites, virus-related diseases and conditions, lichenoid eruptions, lymphoid-related diseases and conditions, melanocytic nevi and neoplasms, monocyte- and macrophage-related diseases and conditions, mucinoses, neurocutaneous, noninfectious immunodeficiency-related diseases and conditions, nutrition-related diseases and conditions, papulosquamous hyperkeratotic related diseases and conditions, pruritic related diseases and conditions, psoriasis (mild, mild to severe, and severe), reactive neutrophilic diseases and conditions, recalcitrant palmoplantar eruptions, diseases and conditions resulting from errors in metabolism, diseases and conditions resulting from physical factors, urticaria and angioedema, vascular-related diseases and conditions, and periodontitis or other diseases and conditions of the gingival.

Skin related diseases and conditions also include skin tumors, pre-malignant tumors, malignant tumors, cell carcinomas, secondary metastasis, radiodermatitis and keratosis.

The healing of wounds can also be assigned to skin diseases and skin related conditions. Wound healing can, hereby, occur at the outer surface of the subject to be treated, at its internal parts, at the skin, eye, nail or nail bed, any surface in the subject's mouth, and at the mucosa, gingival, epithelial surface of the vascular system or other part of the subjects body.

Preference is given to the treatment and/or prophylaxis and/or diagnosis of skin diseases and/or cosmetic skin conditions selected from acne, psoriasis, eczema, dermatitis, atopic dermatitis, atopic eczema, edema, vitiligo, skin ageing, skin, wrinkles, skin desensibilization, Bowens disease, tumors, pre-malignant tumors, malignant tumors, basal cell carcinomas, squamous cell carcinomas, secondary metastases, cutaneous T-cell lymphomas, solar keratosis, arsenical keratosis, radiodermatitis, skin redness, comedo, and cellulite.

The QD-LEC(s) and devices according to the present invention can be used in cosmetics for skin care and skin repair, e.g. as light plaster. The wavelengths or range of wavelengths emitted by said QD-LEC(s) and/or devices is in the range between 400 and 800 nm, preferably between 450 and 750 nm, particularly preferably between 500 and 700 nm, and very particularly preferably between 580 and 640 nm.

Preferred skin diseases and skin-related conditions are selected from acne, psoriasis, eczema, edema, dermatitis, atopic dermatitis, vitiligo, Bowens disease, tumors, pre-malignant tumors, malignant tumors, basal cell carcinomas, squamous cell carcinomas, secondary metastases, cutaneous T-cell lymphomas, solar keratosis, arsenical keratosis, radiodermatitis, and cellulite

Further preferred skin diseases and skin-related conditions are selected from psoriasis, polymorphous light eruption, solar urticaria, actinic reticuloid atopic eczema, vitiligo, pruritus, lichen planus, early cutaneous T-cell lymphoma, dermographism, and pityriasis lichenoides. Preferably theses diseases and conditions are treated with light having a wavelength or a range of wavelengths between 200 and 500 nm, particularly preferably between 250 and 400 nm, and very particularly preferably between 270 and 350 nm.

The said QD-LEC(s) and/or devices can be used for PUVA therapy. PUVA therapy is derived from the therapeutic application of psoralen (7H-furo[3,2-g]chromen-7-one) and derivatives thereof together with UV-A light. PUVA can be employed for the treatment of skin diseases characterized by hyperproliferative conditions. Psoralen is the parent compound in a family of natural products. It is structurally related to coumarines and can preferably be used for the treatment of psoriasis, eczema, vitiligo, mycosis fungoides, cuntaneous T-cell lymphoma, and other autoimmune diseases.

With PUVA can also bet treated atopic eczema, lichen planus, urticaria pigmentosa, polymorphous light eruption, and alopecia greata.

Psoralen can be administered orally or topically to the skin. Preferred compounds are psoralen, 8-methoxypsoralen (8-MOP), 5-methoxypsoralen (5-MOP), and 4,5′,8-trimethylpsoralen (TMP). After oral administration of 8-MOP, patients become gradually reactive to UV-A and therefore to photochemotherapeutic treatment. The patients are maximally reactive 2 to 3 hours after ingestion of the drug, and during this period the irradiation is carried out.

In the case of vitiligo khellin can be used instead of psoralen. The combined treatment with light and khellin is often called KUVA.

The QD-LEC(s) and/or devices of the present invention can also be used for photopheresis. Photophoreresis is a process by which peripheral blood is exposed in an extracorporeal flow system to photoactivate 5-MOP and represents a treatment for disorders caused by aberrant T lymphocytes. It is a therapy for advanced cutaneous T-cell lymphoma, pemphigus vulgaris and progressive systemic sclerosis (scleroderma). It can be used to treat autoimmune disorders. Further diseases that can be treated include multiple sclerosis, organ transplant rejection, rheumatoid arthritis, and AIDS.

The present invention particularly refers to QD-LEC(s) and or devices according to the present invention for the treatment of acneform eruptions. The term acneform eruption refers to a group of dermatoses including acne vulgaris, rosacea, folliculitis, and perioral dermatitis. Acneform eruptions are, generally spoken, caused by changes in the pilosebaceous unit and are selected from acne aestivalis (Mallorca acne), acne conglobata, acne cosmetica, acne fulminans (acute febrile ulcerative acne), acne keloidalis (acne keloidalis nuchae, dermatitis papillaris capillitii, folliculitis keloidalis, folliculitis keloidis nuchae, nuchal keloid acne), acne mecánica, acne medicamentosa, acne miliaris necrotica (acne varioliformis), acne vulgaris, acne with facial edema (solid facial edema), acneform eruptions, blepharophyma, erythrotelangiectatic rosacea (erthemaotelangiectatic rosacea), excoriated acne (acne excoriée des jeunes files, Picker's acne), glandular rosacea, gnathophyma, gram-negative rosacea, granulomatous facial dermatitis, granulomatous perioral dermatitis, halogen acne, hidradenitis suppurativa (acne inversa, Verneuii's disease), idiopathic facial aseptic granuloma, infantile acne, lupoid rosacea (granulomatous rosacea, micropapular tuberculid, rosacea-like tuberculid of Lewandowsky), lupus miliaris disseminatus faciei, metophyma, neonatal acne (acne infantum, acne neonatorum), occupational acne, ophthalmic rosacea (ocular rosacea, ophthalmorosacea), otophyma, persistent edema of rosacea (chronic upper facial erythematous edema, Morbihan's disease, Rosaceous lymphedema), pomade acne, papulopustular rosacea, perifolliculitis capitis abscedens et suffodiens (dissecting cellulitis of the scalp, dissecting folliculitis, perifolliculitis capitis abscedens et suffodiens of Hoffman), perioral dermatitis, periorbital dermatitis (periocular dermatitis), pyoderma faciale (rosacea fulminans), rhinophyma, rosacea (acne rosacea), rosacea conglobata, rosacea fulminans, SAPHO syndrome, steroid rosacea, tropical acne.

Acne vulgaris (commonly called acne) is a common skin condition, caused by changes in pilosebaceous units, skin structures consisting of a hair follicle and its associated sebaceous gland, via androgen stimulation. It is characterized by noninflammatory follicular papules or comedones and by inflammatory papules, pustules, and nodules in its more severe forms. Acne vulgaris affects the areas of skin with the densest population of sebaceous follicles; these areas include the face, the upper part of the chest, and the back. Severe acne is inflammatory, but acne can also manifest in noninflammatory forms. Acne lesions are commonly referred to as pimples, blemishes, spots, zits, or simply acne.

Acne occurs most commonly during adolescence, affecting more than 89% of teenagers, and frequently continues into adulthood. In adolescence, acne is usually caused by an increase in male sex hormones, which people of both genders accrue during puberty. For most people, acne diminishes over time and tends to disappear—or at the very least decrease—after one reaches one's early twenties. There is, however, no way to predict how long it will take to disappear entirely, and some individuals will carry this condition well into their thirties, forties and beyond.

The face and upper neck are the most commonly affected, but the chest, back and shoulders may have acne as well. The upper arms can also have acne, but lesions found there are often keratosis pilaris. Typical acne lesions are comedones, inflammatory papules, pustules and nodules. Some of the large nodules are also called cysts and the term nodulocystic has been used to describe severe cases of inflammatory acne.

Aside from scarring, its main effects are psychological, such as reduced self-esteem and, in some cases, depression or suicide. Acne usually appears during adolescence, when people already tend to be most socially insecure. Early and aggressive treatment is therefore advocated by some to lessen the overall impact to individuals.

Light exposure can be used as treatment for acne. Used twice weekly, this has been shown to reduce the number of acne lesions by about 64% and is even more effective when applied daily. The mechanism appears to be that a porphyrin (Coproporphyrin III) produced within P. acnes generates free radicals when irradiated by 420 nm and shorter wavelengths of light. Particularly when applied over several days, these free radicals ultimately kill the bacteria. Since porphyrins are not otherwise present in skin, and no UV light is employed, it appears to be safe.

The treatment apparently works even better if used with a mixture of the violet/blue light and red visible light (e.g. 660 nm) resulting in a 76% reduction of lesions after three months of daily treatment for 80% of the patients; and overall clearance was similar or better than benzoyl peroxide. Unlike most of the other treatments few if any negative side effects are typically experienced, and the development of bacterial resistance to the treatment seems very unlikely. After treatment, clearance can be longer lived than is typical with topical or oral antibiotic treatments; several months is not uncommon. In addition, basic science and clinical work by dermatologists has produced evidence that intense blue/violet light (405 to 425 nm) can decrease the number of inflammatory acne lesion by 60 to 70% in four weeks of therapy, particularly when the P. acnes is pre-treated with delta-aminolevulinic acid (ALA), which increases the production of porphyrins.

The present invention therefore also relates to a combination of the said QD-LEC(s) or said devices and active drugs or active ingredients for the treatment of therapeutic diseases and/or cosmetic conditions. In particular, the present invention relates to the combined use of said QD-LEC(s) and drugs used for the treatment of acne. The drugs can be selected from any drugs typically employed in order to treat acne, such as antibiotics (topical and/or oral), hormonal treatments, topical retinoids, topical bactericidals, sulfur. Suitable topical bactericidals are, for example, benzoyl peroxide, triclosan, and chlorhexidine gluconate. Suitable topical antibiotics are, for example, erythromycin, clindamycin, and tetracycline. Suitable oral antibiotics are, for example, erythromycin, tetracycline antibiotics (e.g. oxytetracycline, doxycycline, minocycline, or lymecycline), trimethoprim, and minocycline.

Suitable hormones are, e.g., selected from estrogen, progestogen, a combination of estrogen and progestogen, cyproterone, oestrogen, a combination of cyproterone and oestrogen, drospirenone, spironolactone, and cortisone. Suitable oral retinoids are, for example, vitamin A derivatives such as isotretinoin (e.g. Accutane, Amnesteem, Sotret, Claravis, Clarus). Suitable topical retinoids are, for example, tretinoin (e.g. Retin-A), adapalene (e.g. Differin), tazarotene (e.g. Tazorac), isotretinoin, and retinol. Further suitable drugs are, e.g. selected from anti-inflammatory drugs.

The QD-LEC(s) according to the present invention and devices comprising them can also be used in combination with dermabrasion to treat or prevent acne. Dermabrasion is a cosmetic medicial procedure in which the surface of the skin is removed by abrasion (sanding).

Hereby any therapeutic strategy is included. The drug, e.g., can be administered first for a specific time period followed by the application of phototherapy using the QD-LEC(s) or said devices according to the present invention. The time gap between both treatments may also vary, depending on the drug, its photoreactivity, individual circumstances of the subject, and the specific disease or condition. Both treatments may also overlap timely either partly or completely. The exact treatment strategy will depend on the individual circumstances and the severity of the disease or condition.

The combination therapy can have a synergistic effect and can reduce the side effects of traditional treatment strategies (e.g. the side effects of tetracyclines). This is due to the fact, that smaller doses of the drugs may be required when following the combined approach as outlined herein.

Comedones, also called blackhead, can also be treated by phototherapy employing the QD-LEC(s) or devices according to the present invention. A comedon is a yellow or blackish bump or plug on the skin. Actually, it is a type of acne vulgaris. Comedones are caused by excess oils that have accumulated in the sebaceous gland's duct. The substance found in these bumps mostly consists of keratin and modified sebum, which darkens as it oxidizes. Clogged hair follicles, where blackheads often occur, reflect light irregularly to produce a comedon. For this reason, the blockage might not necessarily look black when extracted from the pore, but may have a more yellow-brown colour as a result of its melanin content.

In contrast, a so called whitehead, which is also called closed comedo, is a follicle that is filled with the same material, sebum, but has a microscopic opening to the skin surface. Since the air cannot reach the follicle, the material is not oxidized, and remains white.

The QD-LEC(s) or devices according to the present invention used for the treatment of acne preferably comprises at least one organic electroluminescent compound which emits light in the range between 350 and 900 nm, preferably between 380 and 850 nm, particularly preferably between 400 and 850 nm, and very particularly preferably between 400 and 800 nm.

Further particularly preferred light for the treatment of acne is blue light. Preferred blue light has emission wavelengths for the treatment of acne are 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429 and 430 nm. For example 414 and 415 nm are particularly suitable in order to kill P. acnes bacteria and to help cure existing blemishes and to prevent further outbreaks.

Studies on the application of phototherapy to treat acne revealed that a combination of different wavelengths or ranges of wavelengths are particularly suitable to treat acne efficiently. Particularly preferred is therefore a combination of red light and blue light to treat acne. The said red light is preferably selected from the range between 590 to 750 nm, particularly preferably between 600 and 720 nm, and very particularly preferably between 620 and 700 nm. Two further preferred wavelengths for the treatment of acne are 633 and 660 nm. The blue light can be selected from the wavelengths as described above.

In the case of comedo QD-LEC(s) comprising light emitting compound(s) emitting light with a wavelength of 500 nm or light in the range between 500 and 700 nm are particularly preferred.

Cellulite describes a condition that is claimed to occur in most women, where the skin of the lower limbs, abdomen, and pelvic region becomes dimpled. The causes of cellulite are poorly understood and may involve changes in metabolism and physiology such as gender specific dimorphic skin architecture, alteration of connective tissue structure, vascular changes and inflammatory processes. A couple of therapies are applied to prevent or to treat cellulite. Heat and the increase of blood flow are two common techniques. Therefore light therapy is considered to be beneficial to individuals suffering from cellulite. QD-LEC(s) and/or devices according to the present invention are suitable for the treatment and/or prophylaxis of cellulite. PDT is also suitable for the treatment and/or prophylaxis of cellulite.

The wavelength for the treatment and/or prophylaxis of cellulite that is to be emitted by the QD-LEC(s) and/or devices according to the present invention is in the range between 400 and 1000 nm, preferably in the range between 400 and 900 nm, particularly preferably between 450 and 900 nm, and very particularly preferably between 500 and 850 nm. The more general term skin ageing refers to both the formation of wrinkles and hyperpigmentation. The signs of ageing of the human skin resulting from the effects on the skin of intrinsic and extrinsic factors are defined by the appearance of wrinkles and fine lines, by the yellowing of the skin which develops a wizened appearance along with the appearance of pigmentation blemishes, by a change in the thickness of the skin, generally resulting in a thickening of the stratum corneum and of the epidermis and a thinning of the dermis, by disorganization of the elastin and collagen fibers which causes a loss of elasticity, of suppleness and of firmness, and by the appearance of telnagiectasia.

Some of these signs are more particularly associated with intrinsic or physiological ageing, that is so to say with “normal” ageing associated with age, whereas others are more specific to extrinsic ageing, that is so to say ageing caused by the environment in general; such ageing is more particularly photo-ageing due to exposure to the sun. Other factors causing ageing of the skin are atmospheric pollution, wounds, infections, traumatisms, anoxia, cigarette smoke, hormonal status, neuropeptides, electromagnetic fields, gravity, lifestyle (e.g. excessive consumption of alcohol), repetitive facial expressions, sleeping positions, and psychological stressors.

The changes in the skin which occur due to intrinsic ageing are the consequence of a genetically programmed sequence involving endogenous factors. This intrinsic ageing in particular causes slowing down of the regeneration of skin cells, which is reflected essentially in the appearance of clinical damage such as a reduction of the subcutaneous adipose tissue and the appearance of fine lines or small wrinkles, and in histopathological changes such as an increase in the number and thickness of the elastic fibers, a loss of vertical fibers from the elastic tissue membrane and the presence of large irregular fibroblasts in the cells of this elastic tissue.

In contrast, extrinsic ageing results in clinical damage such as thick wrinkles and the formation of flabby and weather-beaten skin, and in histopathological changes such as an excessive accumulation of elastic substance in the upper dermis and degeneration of the collagen fibers.

There are different biological and molecular mechanisms which are responsible for he ageing of the skin and the process is currently not fully understood. However, it was recognized that both intrinsic and extrinsic factors of ageing of the skin share common mechanisms [P. U. Giacomoni et al., Biogerontology 2004, 2, 219-229]. These factors trigger a process leading to the accumulation of damages in the skin resulting in skin ageing since the expression of cell adhesion molecules provokes recruitment and diapedesis of circulating immune cells, which digest the extracellular matrix (ECM) by secreting collagenases, myeloperoxidases and reactive oxygen species.

The activation of these lytic processes provokes random damage of these resident cells, which in turn secrete prostaglandins and leukotrienes. These signaling molecules induce the degranulation of resident mast cells which release the autacoid histamine and the cytokine TNFalpha thus activating endothelial cells lining adjacent capillaries which release P-selectin and the synthesis of cell adhesion molecules such as E-selectin and ICAM-1. This closes a self-maintained micro-inflammatory cycle, which results in the accumulation of ECM damage, i.e. skin ageing.

There is a strong cosmetic and therapeutic need for novel strategies for the treatment or prophylaxis of skin ageing. Various cosmetic and therapeutic compositions (including for skin care) intended inter alia to prevent or treat ageing of the skin are known. Retinoic acid and derivatives thereof have been described as anti-ageing agents in skin care, cosmetic, or dermatological compositions, in particular in U.S. Pat. No. 4,603,146. Hydroxy acids such as lactic acid, glycolic or alternatively citric acid are also known for this same application, these acids have been described in numerous patents and publications (e.g. EP-A-413528) and introduced into numerous skin care, cosmetic, or dermatological compositions on the market. Aromatic orthohydroxy acids such as salicylic acid have also been proposed (e.g. WO 93/10756 and WO 93/10755).

All of these compounds act against ageing of the skin by desquamation, that is to say removal of the dead cells at the surface of the stratum corneum. This desquamation is also referred to as a keratolytic property. However, these compounds also have side effects, consisting of stinging and redness, which the user finds unpleasant. Thus, there remains a need for anti-ageing methods which are at least as effective as the known compounds, but do not exhibit their drawbacks. Unlike the established strategies to treat or prevent skin ageing, modulating the selectin function is a novel concept intervening the micro-inflammation cascade at a very early stage and treating and preventing intrinsic and extrinsic skin ageing according to the present inventions represents a strategy without the drawbacks known from other strategies.

Phototherapy provides a new way to treat ageing of the skin. Thus, another subject of the invention is the use of the QD-LEC(s) and/or devices according to the present invention for the treatment and/or prophylaxis of skin ageing. This means, that the present invention provides solutions, inter alia, for skin rejuvenation and to reduce or prevent the formation of wrinkles.

The wavelength for the treatment of skin ageing that is to be emitted by the QD-LEC(s) and/or devices according to the present invention is in the range between 400 and 950 nm. Preferably the wavelength is in the range between 550 and 900 nm, and particularly preferably between 550 and 860 nm.

The QD-LEC(s) and/or devices of the present invention may also emit light of different wavelengths or wavelength ranges which also applies for other embodiments of the present invention.

In another preferred embodiment of the present invention the QD-LEC(s) and/or devices used for the treatment of skin ageing emits light in the range of 600 nm and 650 nm, particularly preferably in the range between 620 nm and 650 nm.

The QD-LEC(s) and/or devices according to the present invention used for the treatment and/or prevention of skin ageing preferably comprises at least one organic electroluminescent compound which emits light in the range between 350 and 950 nm, preferably between 380 and 900 nm, and particularly preferably between 400 and 900 nm.

Further particularly preferred light for the treatment and/or prophylaxis of skin ageing is blue light. Preferred blue light has emission wavelengths for the treatment and/or prophylaxis of skin ageing are 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, and 430 nm. For example 415 nm is particularly suitable.

Further particular preferred light for the treatment and/or prophylaxis of skin ageing has a wavelength between 400 and 900 nm.

Skin rejuvenation can also be achieved with light of the wavelength of 830 nm or slightly below or above that value. Therefore, QD-LEC(s) and/or devices according to the present invention emitting light in the range between 700 nm and 1000 nm, preferably between 750 nm and 900 nm, particularly preferably between 750 nm and 860 nm, and very particularly preferably between 800 nm and 850 nm are also subject of the present invention.

Redness of the skin of a subject can be treated by a QD-LEC(s) and/or devices according to the present invention. The wavelength for the treatment and/or prophylaxis of redness that is to be emitted by the QD-LEC(s) and/or devices according to the present invention is in the range between 460 and 660 nm. Preferably the wavelength is in the range between 500 and 620 nm, and particularly preferably between 540 and 580 nm. One particular preferred wavelength for this purpose is 560 nm. Dermatitis of a subject can be treated by a QD-LEC(s) and/or devices according to the present invention. The wavelength for the treatment and/or prophylaxis of dermatitis that is to be emitted by the QD-LEC(s) and/or devices according to the present invention is in the range between 470 and 670 nm. Preferably the wavelength is in the range between 490 and 650 nm, and particularly preferably between 530 and 610 nm. Two particular preferred wavelengths for this purpose are 550 nm and 590 nm.

Atopic eczema of a subject can be treated by a QD-LEC(s) and/or devices according to the present invention. The wavelength for the treatment and/or prophylaxis of atopic eczema that is to be emitted by the QD-LEC(s) and/or devices according to the present invention is in the range between 470 and 670 nm. Preferably the wavelength is in the range between 490 and 650 nm, and particularly preferably between 530 and 610 nm. One particular preferred wavelength for this purpose is 320 nm.

Psoriasis can be treated by a QD-LEC(s) and/or devices according to the present invention. The wavelength for the treatment and/or prophylaxis of psoriasis that is to be emitted by the QD-LEC(s) and/or devices according to the present invention is in the range between 240 and 500 nm. Preferably the wavelength is in the range between 290 and 400 nm, and particularly preferably between 300 and 330 nm. Two particular preferred wavelengths for this purpose are 311 and 320 nm.

Vitiligo can be treated by a QD-LEC(s) and/or devices according to the present invention. The wavelength for the treatment and/or prophylaxis of vitiligo that is to be emitted by the QD-LEC(s) and/or devices according to the present invention is in the range between 240 and 500 nm. Preferably the wavelength is in the range between 290 and 400 nm, and particularly preferably between 300 and 330 nm. One particular preferred wavelength for this purpose is 311 nm.

Targeted phototherapy has enabled therapeutic dosing of ultraviolet light to specific dermatoses while minimizing exposure of healthy skin. Specifically, the 308 nm wavelength of light within the ultraviolet B range has been shown as particularly effective for many dermatoses, including vitiligo; psoriasis; and leukoderma such as that associated with scars, striae alba and post-CO₂ laser resurfacing.

The QD-LEC(s) and/or devices of the present invention can also be used for the treatment of edema. Edema, formerly known as dropsy or hydropsy, is an abnormal accumulation of fluid beneath the skin or in one or more cavities of the body. Generally, the amount of interstitial fluid is determined by the balance of fluid homeostasis, and increased secretion of fluid into the interstitium or impaired removal of this fluid may cause edema. Five factors can contribute to the formation of edema: (1) It may be facilitated by increased hydrostatic pressure or by reduced oncotic pressure within blood vessels or (2) by increased blood vessel wall permeability as in inflammation or (4) by obstruction of fluid clearance via the lymphatic or (5) by changes in the water retaining properties of the tissues themselves. Raised hydrostatic pressure often reflects retention of water and sodium by the kidney.

The QD-LEC(s) and/or devices according to the present invention used for the treatment of edema preferably emit light in the range between 760 and 940 nm, preferably between 780 and 920 nm, particularly preferably between 800 and 900 nm, and very particularly preferably between 820 and 880 nm.

One further particularly preferred emission wavelength for the treatment of edema is 850 nm.

Another subject of the present invention relates to QD-LEC(s) and/or devices according to the present invention for the treatment and/or prophylaxis of infections and inflammatory, neurological, and psychological diseases and/or conditions.

Many inflammatory diseases, disorder, and conditions can be treated with phototherapy. QD-LEC(s) and/or devices according to the present invention for the treatment and/or prophylaxis of inflammatory disorders is also subject of the present invention. Inflammatory diseases and conditions cover a wide range of indications. Many diseases or conditions which are seemingly unrelated to inflammation have inflammatory components that can be treated with the QD-LEC(s) and/or devices according to the present invention. The skin diseases and conditions mentioned in the present invention all have inflammatory components, such as acne, psoriasis, atopic dermatitis, eczema. A non limiting selection of further inflammatory diseases and conditions that can be treated with QD-LEC(s) and/or devices according to the invention is arthritis, inflammatory bowel disease, gingival inflammation, inflammation of the mucosa, inflammation of the nail bed, arteriosclerosis, and inflammation of the vascular system.

Preferred wavelengths for the treatment and/or prophylaxis of inflammation are in the range between 350 and 900 nm, particularly preferably between 380 and 900 nm, and very particularly preferably between 400 and 860 nm. Further preferred wavelengths for the treatment and/or prophylaxis of inflammation are 405, 420, and 850 nm.

Said QD-LEC(s) and/or devices can be used for the treatment and/or prophylaxis of infections. Infections can be caused by bacteria and viruses.

Light has several positive effects on infections. Light has, e.g., anti-inflammatory effects through the stimulation of the tissue as outlined elsewhere within the present invention.

Phototherapy with QD-LEC(s) and/or devices according to the present invention is beneficial for the use to treat wounds. Wound healing is often associated with inflammation. Therefore the same wavelengths and ranges of wavelengths as outlined for the treatment and/or prophylaxis of inflammation can be applied. Treating wounds by phototherapy also prevents the formation of scares. Particularly preferred wavelengths for the treatment and/or prophylaxis of wounds and/or scares are in the range between 600 and 950 nm and very particularly preferably between 650 and 900 nm. Further preferred wavelengths for the treatment and/or prophylaxis of wounds and scares are 660, 720, and 880 nm.

Other infections that can efficiently be treated with QD-LEC(s) and/or devices according to the present invention are caused by bacteria.

Further infections that can efficiently be treated with QD-LEC(s) and/or devices according to the present invention are caused by viruses. A preferred embodiment of this invention is the use of the said QD-LEC(s) and/or devices for the treatment and/or prophylaxis of viral infections particularly caused by cytomegalovirus (CMV), encephalo myocarditis virus (EMCV), poliovirus, influenza virus, parainfluenza respiratory influenza virus, respiratory syncytial virus, Japanese encephalitis virus, Dengue virus, hepatitis A virus (HAV), hepatitis B virus (HBV), hepatitis C virus (HCV), hepatitis D virus (HDV), hepatitis E virus (HEV), hepatitis F virus (HFV), hepatitis G virus (HGV) Epstein Barr Virus (EBV), human immunodeficiency virus type 1 (HIV-I), human immunodeficiency virus type 2 (HIV-2), varicella zoster virus, herpes simplex virus, in particular herpes simplex virus type 1 (HSV-I), herpes simplex virus type 2 (HSV-2), or human herpes virus 1, 2, 3, 4, 7, or 8, Kaposi's sarcoma-associated herpesvirus (KSHV), rotavirus, papilloma virus, and human papilloma virus (HPV), in particular HPV of the types: 1, 2, 3, 4, 5, 8, 9, 11, 12, 13, 14, 15, 16, 17, 18, 19-29, 31, 32, 34, 36-38, 46-50, 56, or 58.

In particular viral skin diseases and/or tumor disorders can be treated with QD-LEC(s) and/or devices according to the present invention such as genital warts, benign tumors of the skin and/or mucosa caused by papilloma viruses, in particular verrucae plantares, verrucae vulgares, verrucae planae juveniles, epidermodysplasia verruciformis, Condylomata acuminate, Condylomata plana, bowenoid papulosis, papilloma on the larynx and oral mucosa, focal epithelial hyperplasia, herpes labialis, varicella and shingles.

In a particularly preferred embodiment of the present invention the QD-LEC(s) and/or devices of the invention can be used for the treatment and/or prophylaxis of warts. Pulsed light therapy might be one way to treat warts with QD-LEC(s) and/or devices according to the present invention.

QD-LEC(s) and/or devices according to the present invention for the treatment and/or prophylaxis of neurological or psychological diseases and/or conditions is also subject of the present invention.

A preferred neurological disease according to the present invention is Morbus Parkinson (MB). When light reaches a certain level of intensity, it inhibits melatonin which in turn limits the production of dopamine. By limiting the melatonin is supposed to lead to a have better production and use of dopamine in the brain. Recent case studies of light therapy on MB patients involving bright light therapy have had positive results with marked improvement in bradykinesia and rigidity in most patients while being exposed for only ninety minutes.

Further preferred neurological and psychological diseases and/or conditions according to the present invention are mood and sleep related. Light is well known to be beneficial on the mood in many circumstances. Phototherapy can also be employed to treat depression, seasonal affective disorder (SAD), non seasonal depression, circadian rhythm sleep disorder (chronic circadian rhythm sleep disorder (CRSD), situational CRSD).

The US National Library of Medicine notes that some people experience a serious mood change when the seasons change. They may sleep too much, have little energy, and crave sweets and starchy foods. They may also feel depressed. Though symptoms can be severe, they usually clear up. The condition in the summer is often referred to as Reverse Seasonal Affective Disorder, and can also include heightened anxiety. It has been estimated that 1.5 to 9% of adults in the US experience SAD.

There are different treatments for classic (winter-based) seasonal affective disorder, including light therapy with bright lights, antidepressant medication, cognitive-behavioral therapy, ionized-air administration, and carefully timed supplementation of the hormone melatonin.

The wavelength for the treatment and/or prophylaxis of these neurological and psychological diseases and/or conditions that is to be emitted by said QD-LEC(s) and/or devices is in the range between 350 and 600 nm. Preferably the wavelength is in the range between 400 and 550 nm, and particularly preferably between 440 and 500 nm. Two particular preferred wavelengths for this purpose are 460 and 480 nm.

The QD-LEC(s) and/or devices according to the present invention may also be used for the treatment and/or prophylaxis of pain. Pain relief by phototherapy is well known. The following conditions produce pain that can be treated successfully with phototherapy: carpal tunnel syndrome, chronic wounds, epicondylitis, headache, migraine, plantar fasciitis, tendonditis and bursitis, neck pain, back pain, muscle pain, trigeminal neuralgia, and Whiplash-associated injuries.

Preferably, muscle pain is treated with QD-LEC(s) and/or devices emitting red or infrared light.

Alopecia greata is a condition affecting humans, in which hair is lost from some or all areas of the body, usually from the scalp. Because it causes bald spots on the scalp, especially in the first stages, it is sometimes called spot baldness. In 1 to 2% of cases, the condition can spread to the entire scalp (alopecia totalis) or to the entire epidermis (alopecia universalis). Conditions resembling alopecia greata, and having a similar cause, occur also in other species.

Alopecia greata (autoimmune hair loss) can be treated by a QD-LEC(s) and/or devices according to the present invention. The wavelength for the treatment and/or prophylaxis of alopecia greata that is to be emitted by the QD-LEC(s) and/or devices according to the present invention is in the range between 240 and 500 nm. Preferably the wavelength is in the range between 290 and 400 nm, and particularly preferably between 300 and 330 nm. One particular preferred wavelength for this purpose is 311 nm.

Said QD-LEC(s) and/or devices used for the disinfection and/or sterilization and/or preservation of beverages and nutrition is also subject of the present invention.

The use of light for the purpose of disinfection and/or sterilization and/or preservation is well known. The QD-LEC(s) and/or devices according to the present invention can be used for this purpose. Hereby any kind of disinfection and/or sterilization and/or preservation is meant and includes without limitation the disinfection of wounds, nutrition, and solid and liquids objects, such cosmetic, medical devices, devices used for surgery and beverages.

Preference is given to QD-LEC(s) and/or devices for the disinfection and/or sterilization and/or preservation of beverages, preferably water, and particularly preferably drinking water. Contaminated water causes many infections worldwide and leads often to severe diseases or death of the individuals.

Water filter systems of commercial providers take advantage of ion exchange technology. The filter, however, tend to microbial contamination, which, in turn results in water which is contaminated with microbes. One solution is to add silver salt which may be from a toxicological point of view problematic. The QD-LEC(s) and/or devices of the present invention provide a solution to this problem. They can be used to be incorporated into the water filter system in order to provide a safe, efficient, and low cost way to provide water with a low degree of microbial contamination. The light source can irradiate both the water before or after filtering or the filter cartridge itself. Preferably the light source comprising the QD-LEC(s) irradiates both the filter cartridge and the already filtered water.

The procedure of disinfection and/or sterilization and/or preservation of water as outlined above can basically be applied to any other liquid, in particular beverage analogously.

Therefore, the QD-LEC(s) and/or devices according to the present invention can be used for the disinfection and/or preservation of beverages and nutrition for humans and animals.

Wavelengths for disinfection and/or sterilization and/or preservation according to the present invention are in the range between 200 nm and 600 nm, preferably between 250 nm and 500 nm, and very particularly preferably between 280 nm and 450 nm.

In another embodiment the present invention relates to the said QD-LEC(s) and/or devices for the application in photodynamic therapy (PDT).

Wavelengths required for PDT according to the present invention are in the range between 300 and 700 nm, preferably between 400 and 700 nm, and very particularly preferably between 500 and 700 nm. Four further preferred wavelengths are 595, 600, 630, and 660 nm.

Any therapy known as PDT can be treated with QD-LEC(s) and/or devices according to the present invention and devices comprising them. In particularly PDT as outlined within the present invention can be treated with QD-LEC(s) and/or devices according to the present invention. The property of dyes with a polycyclic hydrocarbon type chemical structure to accumulate in greater amounts in tumor tissues than in normal tissues is well known. The dyes include acridines, xanthenes, psoralens, and porphyrins. The latter dyes, in particular, hematoporphyrin (Hp) and some of its chemical derivatives (e.g. Hp D, wherein Hp D is a mixture of Hp derivatives), have superior tumor-localizing properties, which are the basis of phototherapeutic treatment of tumors with red light irradiation at predetermined times after systemic administration of the drug.

Drug used for PDT are preferably selected from aminolevulinic acid/methyl aminolevulinate, efaproxiral porphyrin derivatives (porfimer sodium, talaporfin, temoporfin, verteporfin).

In a further embodiment the present invention relates to the said QD-LEC(s) and/or devices for the treatment and/or prophylaxis of jaundice and crigler naijar, preferably jaundice.

Jaundice, which is also known as icterus, is a yellowish discoloration of the skin, the conjunctival membranes over the sclerae (whites of the eyes), and other mucous membranes. The discoloration is caused by hyperbilirubinemia (increased levels of bilirubin in the blood). This hyperbilirubinemia subsequently causes increased levels of bilirubin in the extracellular fluids. Jaundice is classified in three groups, pre-hepatic (hemolytic) jaundice, hepatic (hepatocellular) jaundice, and post-hepatic (obstructive) jaundice.

Pre-hepatic jaundice is caused by anything which causes an increased rate of hemolysis, i.e. breakdown of red blood cells. In tropical countries, malaria can cause jaundice in this manner. Certain genetic diseases, such as sickle cell anemia, spherocytosis and glucose 6-phosphate dehydrogenase deficiency can lead to increased red cell lysis and therefore hemolytic jaundice. Commonly, diseases of the kidney, such as hemolytic uremic syndrome, can also lead to coloration. Defects in bilirubin metabolism also present as jaundice. Jaundice usually comes with high fevers. Rat fever (leptospirosis) can also cause jaundice.

Hepatic jaundice causes include acute hepatitis, hepatotoxicity and alcoholic liver disease, whereby cell necrosis reduces the liver's ability to metabolise and excrete bilirubin leading to a buildup in the blood. Less common causes include primary biliary cirrhosis, Gilbert's syndrome (a genetic disorder of bilirubin metabolism which can result in mild jaundice, which is found in about 5% of the population), Crigler-Najjar syndrome, metastatic carcinoma and Niemann-Pick disease, type C. Jaundice seen in the newborn, known as neonatal jaundice, is common, occurring in almost every newborn as hepatic machinery for the conjugation and excretion of bilirubin does not fully mature until approximately two weeks of age.

Post-hepatic jaundice, also called obstructive jaundice, is caused by an interruption to the drainage of bile in the biliary system. The most common causes are gallstones in the common bile duct, and pancreatic cancer in the head of the pancreas. Also, a group of parasites known as “liver flukes” can live in the common bile duct, causing obstructive jaundice. Other causes include strictures of the common bile duct, biliary atresia, ductal carcinoma, pancreatitis and pancreatic pseudocysts. A rare cause of obstructive jaundice is Mirizzi's syndrome.

Jaundice, in particular neonatal jaundice, can lead to severe medical consequences if not or not appropriately treated. Increased concentrations of bilirubin can result in a brain-damaging condition known as kernicterus, leading to significant lifelong disability; there are concerns that this condition has been rising in recent years due to inadequate detection and treatment of neonatal hyperbilirubinemia. Early treatment often consists of exposing the infant to intensive phototherapy in an more or less isolated incubator. The therapy often represents an emotionally or psychologically difficult situation for both the infant and the parents. The QD-LEC(s) and/or devices of the present invention can be employed in order to provide flexible and ambulatory devices such as blankets. Thus, the infant can be treated while laying in its parents' arms. Traditional therapies also easily lead to overheating of the infant, which can also be significantly reduced with the QD-LEC(s) and/or devices of the present invention and devices comprising them.

Preferably the present invention relates to QD-LEC(s) and/or devices used for the treatment of neonatal jaundice.

Jaundice of a subject can be treated by QD-LEC(s) and/or devices according to the present invention. The wavelength for the treatment and/or prophylaxis of jaundice that is to be emitted by the QD-LEC(s) and/or devices according to the present invention is in the range between 300 and 700 nm. Preferably the wavelength is in the range between 350 and 600 nm, and particularly preferably between 370 and 580 nm. Further preferred wavelengths are in the range between 400 and 550 nm. Particularly preferred wavelengths are in the range between 410 and 470 nm. Two particular preferred wavelengths for this purpose are 450 and 466 nm.

In another embodiment the present invention relates to the use of the QD-LEC(s) for the preparation of a device for the treatment and or/prophylaxis of therapeutic diseases and/or cosmetic conditions. The therapeutic diseases and conditions are the same as the ones described elsewhere in the present invention.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

It will be appreciated that many of the features described above, particularly of the preferred embodiments, are inventive in their own right and not just as part of an embodiment of the present invention. Independent protection may be sought for these features in addition to or alternative to any invention presently claimed.

The teaching as disclosed here can be abstracted and combined with other examples disclosed.

Other features of the invention will become apparent in the course of the following description of exemplary embodiments and drawings, which are given for illustration of the invention and are not intended to be limiting thereof.

### BRIEF DESCRIPTION OF THE DRAWINGS

FIG. 1: Device structure for a QD-LEC, with substrate (101), anode (102), buffer layer or HIL (103), interlayer (104), EML (105) and cathode (106).

FIG. 2: Schema for the preparation of the QD-LEC on flexible substrate.

FIG. 3: Attachment of printed battery to plaster comprising QD-LEC.

### WORKING EXAMPLES

### Example 1

### Materials

The following materials will be used in the present invention as examples.

Quantum dot (QD1) is a core-shell type quantum-dot by Plasmachem GmbH, Berlin, Germany, having a CdSe spheric core capped with epitaxial ZnS shell. QD1 has a hydrophobic surface layer comprising mostly trioctylphosphine oxide. The photoluminescent quantum efficiency (PLQE) of QD1 is measured using Rhodamine 6G as reference and is found to be about 30%.

### Triplet Green Emitter TEG1:

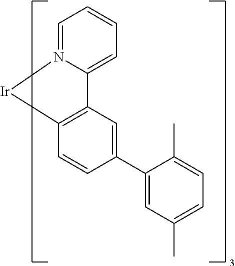

### Triplet Matrix Material TMM1

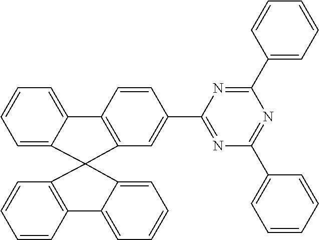 [SMILES: c1ccc(-c2nc(-c3ccccc3)nc(-c3ccc4c(c3)C3(c5ccccc5-c5ccccc53)c3ccccc3-4)n2)cc1]

### Triplet Matrix Material TMM2

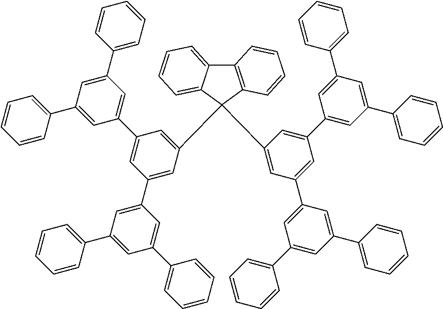 [SMILES: c1ccc(-c2cc(-c3ccccc3)cc(-c3cc(-c4cc(-c5ccccc5)cc(-c5ccccc5)c4)cc(C4(c5cc(-c6cc(-c7ccccc7)cc(-c7ccccc7)c6)cc(-c6cc(-c7ccccc7)cc(-c7ccccc7)c6)c5)c5ccccc5-c5ccccc54)c3)c2)cc1]

### Matrix for Singlet System SMB1

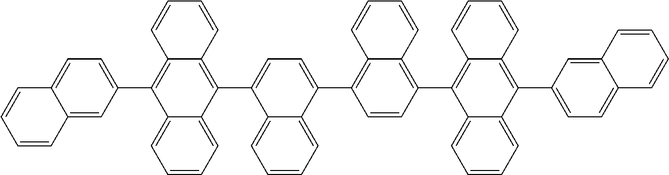 [SMILES: c1ccc2cc(-c3c4ccccc4c(-c4ccc(-c5ccc(-c6c7ccccc7c(-c7ccc8ccccc8c7)c7ccccc67)c6ccccc56)c5ccccc45)c4ccccc34)ccc2c1]

### Singlet Blue Emitter SEB1

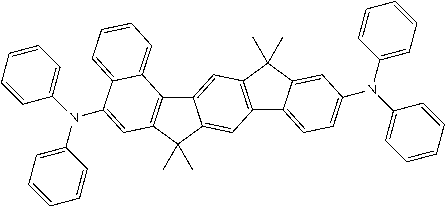 [SMILES: CC1(C)c2cc(N(c3ccccc3)c3ccccc3)ccc2-c2cc3c(cc21)-c1c(cc(N(c2ccccc2)c2ccccc2)c2ccccc12)C3(C)C]

Poly(ethylene oxide) (PEO, M_{w}=5×10⁶ g/mol, Aldrich) is used as ion conductor; and Lithium trifluoromethane sulfonate (LiTrf, 99.995% metal basis; Aldrich) as ion source.

HIL-012 is a hole transport and electron blocking material, and is used as interlayer (IL).

### Example 2

### Preparation of QD-LEC from Solution

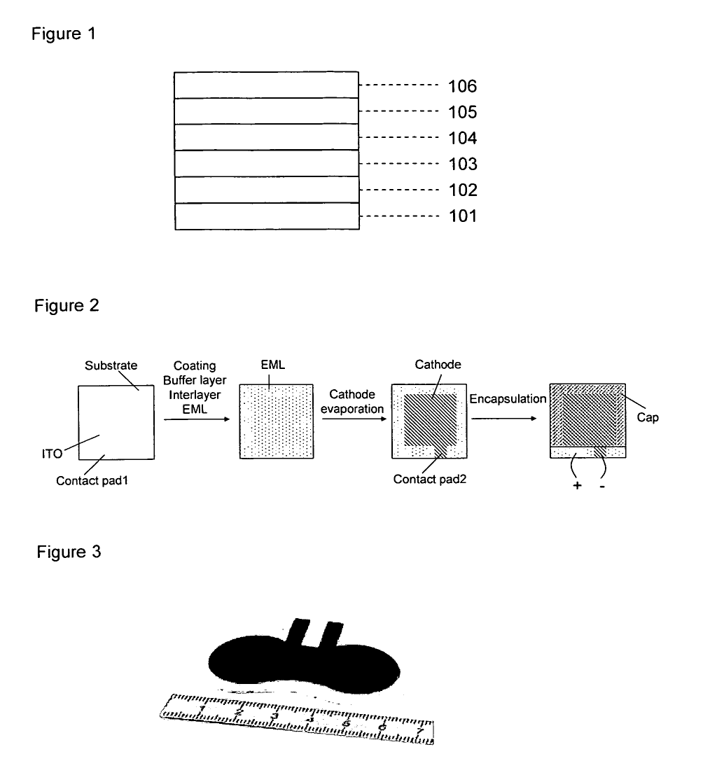

QD-LECs with the structure Cathode/EML/Interlayer/HIL/ITO, as shown in FIG. 1, is prepared according to the following procedure:
1. Deposition of 80 nm PEDOT (Baytron P AI 4083) as hole injection layer (HIL) onto an ITO coated glass substrate by spin coating.
2. Deposition of 20 nm interlayer by spin coating from toluene solution of HIL-012 having a concentration of 0.5% wt/l in glovebox.
3. Heating interlayer layer at 180° C. for 1 h in glovebox.
4. Deposition of emissive layer (EML) from a chlorbenzene solution to a thickness of 250 nm by using doctor blade technique (alternatively dip-coating can also be used); the materials of the EMLs, the corresponding solutions and the thickness of the EMLs are listed in Table 1. Spin-coating is not the optimal method to coat EMLs. This is, because the quantum dots have a much higher molecular weight as compared to other organic compounds, most of them may be lost by the centrifugal force during the spin-coating.
5. Heating the device to remove the residual solvent; the heating condition for both device is 30 minutes at 60° C. Heat-treatment shouldn't lead to re-crystallization in EML.
6. Deposition a cathode (150 nm Al) over the EML by vacuum thermal evaporation;
7. Encapsulation of the device using UV-curved epoxy resin (UV Resin T-470/UR7114, Nagase Chemtex Corporation) and a glass cap.

TABLE 1

| | | | EML |
|---|---|---|---|
| | Composition for EML | Conc. | thickness |
| | [wt %] | [mg/ml] | [nm] |
| QD-LEC1 | 13.5% TMM1:13.5% TMM2:9% | 24 | 250 |
| | TEG1: 36% PEO:8% LiTrf:20% QD1 | | |
| QD-LEC2 | 40.5% SMB1:5% SEB1: | 16 | 250 |
| | 40.5% PEO:9% LiTrf:5% QD1 | | |

Conc.: concentration

### Example 3

### Measurements and Comparison of Results

QD-LEC is characterized by the determination of the following properties: VIL characteristics, EL spectrum and color coordinates, efficiency, driving voltages.

The performance of QD-LECs is summarized in the Table 2, wherein Uon stands for turn-on voltage, U(100) for the voltage at 100 nits.

| | Max. Eff. | Uon | U(100) | CIE @ |
|---|---|---|---|---|
| Device | [cd/A] | [V] | [V] | 100 cd/m² |
| QD-LEC1 | 1.8 | 3.2 | 4.5 | 0.67/0.33 |
| QD-LEC2 | 0.9 | 3.4 | 5.2 | 0.67/0.33 |

### Example 4

### Flexible Red QD-LEC

The preparation of the flexible light emitting devices QD-LEC3 having the same EML as QD-LEC1 and QD-LEC4 having the same EML as QD-LEC2 is as follows and shown in FIG. 2.
1. 150 nm ITO is sputtered on PEN using a mask, as shown in FIG. 2. The dimension of the substrate (PEN) and the emissive area is 3×3 cm and 2×2 cm, respectively.
2. see step 2 in Example 2
3. see step 3 in Example 2
4. see step 4 in Example 2
5. see step 5 in Example 2
6. The device is encapsulated. Encapsulation of the light emitting devices is achieved using a UV-cured resin, UV Resin T-470/UR7114 (Nagase Chemtex Corporation), and a PEN cap, which is smaller than the substrate to leave the contact pads free, as shown in step 4 of FIG. 2. The UV-resin is applied at first on the edge of the pixel, and the cap is then located on top of them. Then the device is exposed to UV light for 30 seconds. All theses steps are performed are in a glove-box.

### Example 5

### Device for Therapeutic and/or Cosmetic Applications

The final devices for using in therapeutic and cosmetic applications can be realised, e.g., by attaching the QD-LEC devices to plasters. The external power source can be applied through the contact pads.

A battery is a preferred power source for the devices, particularly preferred is the printed thin film battery for light weight. The printed thin film battery can be acquired, e.g., from Fraunhofer Institute, as shown in FIG. 3.

In some treatments, the device should be driven in pulse mode. Therefore a controller, particularly a pocket portable one, for pulse driving, can be used. This can be realised by using a commercially available flasher unit or blinker unit. Further such flasher unit can be integrated in the power unit, according to the principle of general trigger circuit, as for example shown in Fachkunde Elektrotechnik, Verlag Europa-Lehrmittel, Nourney, Vollmer GmbH & Co., 5657 Haan-Gruiten, 227.

### Example 6

### Treatment of Crow's Feet

QD-LEC1 is used for the treatment and/or prophylaxis of wrinkles. A plaster is prepared according to Example 5 having a printed battery as power supply. The battery on each plaster supplies energy for a irradiation time of 30 min.

A 22-week pilot study with 15 female human subjects in the age between 30 and 40 years is conducted according to standard methods well known to the person skilled in the art. One of the main selection criteria for the inclusion within the study is the occurrence of crown's feet with almost equal manifestation on both sides of the face, i.e. in proximity to the left and right eye. Each subject is treated on the right hand side with a plaster comprising QD-LEC1 for 30 min. every second day for 22 weeks. Comparison of the skin in proximity of the left eye and right eye reveals a significant improvement of the skin on the treated side. The crow's feet are shorter and less deep. The skin treated with light emitted by the QD-LEC device appears smoother as compared to the untreated skin.

1-18. (canceled)

19. A light emitting electrochemical cell (QD-LEC) comprising at least one quantum dot, at least one ionic compound, and at least one small molecule organic functional material selected from host materials, fluorescent emitters, phosphorescent emitters, hole transport materials (HTMs), hole injection materials (HIMs), electron transport materials (ETMs), and electron injection materials (EIMs).

20. The QD-LEC according to claim 19, wherein the at least one small molecule organic functional material is a fluorescent emitter.

21. The QD-LEC according to claim 19, wherein the at least one small molecule organic functional material is a phosphorescent emitter.

22. The QD-LEC according to claim 19 comprising
(1) a first electrode;
(2) a second electrode; and
(3) an emissive layer (EML) comprising at least one quantum dot, at least one ionic compound, and at least one small organic functional material positioned between the first and second electrode.

23. The QD-LEC according to claim 19, wherein the quantum dot is selected from Group II-VI, Group III-V, Group IV-VI and Group IV semiconductors, or a combination thereof.

24. The QD-LEC according to claim 19, wherein the quantum dot is ZnO, ZnS, ZnSe, ZnTe, CdS, CdSe, CdTe, HgS, HgSe, HgTe, MgS, MgSe, GeS, GeSe, GeTe, SnS, SnSe, SnTe, PbO, PbS, PbSe, PbTe, GaN, GaP, GaAs, GaSb, InN, InP, InAs, InSb, AlN, AlP, AlAs, AlSb, GaN, GaP, GaAs, GaSb, or a combination thereof.

25. The QD-LEC according to claim 19, wherein the ionic compound comprises a ionic transition metal complex (iTMC).

26. The QD-LEC according to claim 19, wherein the emissive layer (EML) comprises at least one ionic quantum dot and at least one electrically neutral small organic functional molecule selected from the group of host materials, fluorescent emitters, phosphorescent emitters, hole transport materials (HTMs), hole injection materials (HIMs), electron transport materials (ETMs), or electron injection materials (EIMs).

27. A device comprising at least one QD-LEC according to claim 19.

28. The device according to claim 27, wherein the device emits electromagnetic irradiation in an area of at least 0.5 cm².

29. The device according to claim 27, wherein the device comprises a power supply or an interface for an external power supply.

30. The device according to claim 27, wherein the device is an ambulatory device and comprises an attachment means for attaching the device to a patient.

31. The QD-LEC according to claim 19 for the treatment and/or prophylaxis and/or diagnosis of diseases and/or cosmetic conditions.

32. A method for the treatment and/or prophylaxis and/or diagnosis of skin diseases and/or cosmetic skin conditions which comprises utilizing the QD-LEC according to claim 19.

33. The method according to claim 32, wherein the treatment and/or prophylaxis and/or diagnosis of skin diseases and/or cosmetic skin conditions selected from acne, psoriasis, eczema, dermatitis, atopic dermatitis, atopic eczema, edema, vitiligo, skin ageing, skin, wrinkles, skin desensibilization, Bowens disease, tumors, pre-malignant tumors, malignant tumors, basal cell carcinomas, squamous cell carcinomas, secondary metastases, cutaneous T-cell lymphomas, solar keratosis, arsenical keratosis, radiodermatitis, skin redness, comedo, and cellulite.

34. A method for the treatment and/or prophylaxis and/or diagnosis of infections and inflammatory, neurological, and psychological diseases and/or conditions which comprises utilizing the QD-LEC according to claim 19.

35. A method for the sterilization and/or disinfection and/or preservation of water, drinking water, soft drinks, beverages, foodstuff, and nutrition which comprises utilizing the QD-LEC according to claim 19.

36. A method for use in application in photodynamic therapy (PDT) and/or for the treatment and/or prophylaxis of jaundice and crigler naijar which comprises utilizing the QD-LEC according to claim 19.

37. A method for the treatment and/or prophylaxis and/or diagnosis of diseases and/or cosmetic conditions, which comprises utilizing the QD-LEC according to claim 19.
